(19) United States
(12) Patent Application Publication
(10) Pub. No.: US 2022/0349017 A1
(43) Pub. Date: Nov. 3, 2022

(54) REAGENTS AND METHODS FOR DETECTING AAV SHEDDING

(71) Applicant: Siemens Healthcare Diagnostics Inc., Tarrytown, NY (US)

(72) Inventors: Arejas J. Uzgiris, Berkeley, CA (US); Isabelle Philipp, Berkeley, CA (US); Xiaolei Qiu, San Ramon, CA (US); Sara Weitz, El Sobrante, CA (US); Zuguang Wang, Hayward, CA (US)

(73) Assignee: Siemens Healthcare Diagnostics Inc., Tarrytown, NY (US)

(21) Appl. No.: 17/597,826

(22) PCT Filed: Aug. 29, 2020
(86) PCT No.: PCT/US2020/048654

§ 371 Date: Jan. 25, 2022

Related U.S. Application Data
(60) Provisional application No. 62/893,486, filed on Aug. 29, 2019.

(51) Int. Cl. C12Q 1/70; C12Q 1/6818

(52) U.S. Cl. CPC C12Q 1/701; C12Q 1/6818

(57) ABSTRACT

The present disclosure provides, among other things, primers and probes for detecting shedding of an AAV construct or fragment thereof in a subject. In some embodiments the primers are selected to generate an amplicon that comprises (i) a first strand comprising (1) a nucleotide sequence corresponding to the forward primer, and (2) a nucleotide sequence corresponding to a portion of the AAV construct or fragment thereof, (ii) a second strand comprising (1) a nucleotide sequence of the reverse primer, and (2) a nucleotide sequence that is complementary to the portion of the AAV construct or fragment thereof, or (iii) a combination thereof, where the portion of the AAV construct or fragment thereof comprises a nucleotide sequence that spans a junction between a regulatory element and the therapeutic gene of interest.

This application incorporates by reference the sequence listing which is submitted together with this application in computer readable form which has the file name 2918P21346WO_SeqListing.txt and is 14.4 KB.

### BACKGROUND

The field of gene therapy is progressing at a tremendous rate. To facilitate this progression, appropriate safety standards must be met. When utilizing viral vectors for the delivery of gene therapy, a key safety variable to measure is the viral particles that are released and/or shed from a patient. There remains a pressing need for accurate and reproducible mechanisms for measuring patient viral shedding titers.

### SUMMARY

The present disclosure provides compositions and methods suitable for the accurate quantification of certain viral particles, for example, in samples from subjects, e.g., humans.

In some embodiments, the present disclosure provides a composition comprising a collection of primers. In some embodiments, a composition comprises a collection of primers and one or more probes.

In some embodiments, a collection of primers comprises one or more forward primers. In some embodiments, at least one forward primer has a nucleotide sequence according to SEQ ID NO: 1 or an active fragment thereof.

In some embodiments, a collection of primers comprises one or more reverse primers. In some embodiments, at least one reverse primer has a nucleotide sequence according to SEQ ID NO: 11 or an active fragment thereof.

In some embodiments, at least one probe has a nucleotide sequence according to SEQ ID NO: 17 or an active fragment thereof.

In some embodiments, a composition comprises a collection of primers and one or more probe sequences, wherein at least one primer has a nucleotide sequence according to SEQ ID NO: 1 or an active fragment thereof, at least one primer has a nucleotide sequence according to SEQ ID NO: 11 or an active fragment thereof, and at least one probe has a nucleotide sequence according to SEQ ID NO: 17 or an active fragment thereof.

In some embodiments, a composition comprises a sample. In some embodiments, a sample is obtained from a subject who has been administered a viral vector. In some embodiments, a viral vector is an AAV construct.

In some a composition comprises a sample obtained from a subject who has been administered a Duchenne Muscular Dystrophy (DMD) AAV construct. In some embodiments, a DMD AAV construct administered to a subject comprises one or more regulatory elements and one or more therapeutic genes of interest. In certain embodiments, a sample obtained from a subject who has been administered a DMD AAV construct comprises nucleic acids comprising said DMD AAV construct or a fragment thereof. In certain embodiments, a DMD AAV construct comprises one or more regulatory elements, wherein the one or more regulatory elements comprise an enhancer, a promoter, a polyA signal sequence, or a combination thereof. In some embodiments, a DMD AAV construct comprises a therapeutic gene of interest comprising microdystrophin.

In some embodiments, a composition described herein further comprises a plurality of amplicons. In some embodiments, an amplicon (e.g., each amplicon) comprises: (a) a first strand comprising: (i) a nucleotide sequence corresponding to the forward primer, and (ii) a nucleotide sequence corresponding to a portion of a DMD AAV construct or fragment thereof; (b) a second strand comprising: (i) a nucleotide sequence of the reverse primer, and (ii) a nucleotide that is complementary to the portion of the DMD AAV construct or fragment thereof; or (c) a combination thereof. In some embodiments, the portion of the DMD AAV construct or fragment thereof comprises a nucleotide sequence that spans a junction between two regulatory elements, a junction between a construct and regulatory element, or a junction between a regulatory element and the therapeutic gene of interest.

In some embodiments, a composition comprising a plurality of amplicons as described herein also comprises at least one or more probe sequences or active fragments thereof capable of hybridizing with the amplicon. In some embodiments, a composition comprising a plurality of amplicons as described herein also comprises at least one or more probe sequences. In some embodiments, at least one probe sequence has a nucleotide sequence according to SEQ ID NO: 17 or an active fragment and is capable of hybridizing with the amplicon. In some embodiments, one or more probe sequences is amenable to detection. In some embodiments, a level of probe or probe-amplicon hybridization is quantifiable (e.g., a level of fluorescence is quantifiable, a level of fluorescence quenching is quantifiable, a level of total amplicon quantity is quantifiable, or a level of free and/or hybridized probe is quantifiable).

In some embodiments, the present disclosure provides a method comprising contacting a sample obtained from a subject with a composition as described herein, wherein the subject has been administered a DMD AAV construct, and wherein the DMD AAV construct comprises one or more regulatory elements and a therapeutic gene of interest.

In some embodiments, a method comprises amplifying a target sequence to generate a plurality of amplicons. In some embodiments, a target sequence is a DMD AAV construct or fragment thereof. In some embodiments, each amplicon comprises: (i) a first strand comprising: (1) a nucleotide sequence corresponding to the forward primer, and (2) a nucleotide sequence that is complementary to the portion of a DMD AAV construct or fragment thereof or (iii) a combination thereof. In some embodiments, a portion of a DMD AAV construct or fragment thereof comprises a nucleotide sequence that spans a junction between two regulatory elements, a junction between a construct and a regulatory element, or a junction between a regulatory element and the therapeutic gene of interest.

In some embodiments of a method described herein, a composition comprises a plurality of probes each with a nucleotide sequence according to SEQ ID NO: 17 or an active fragment thereof. In some embodiments, a composition comprises a plurality of probes and at least one a subset of the plurality of probes comprises a nucleotide sequence according to SEQ ID NO: 17 or an active fragment thereof.

In some embodiments, a method described herein comprises detecting a level of hybridization between a plurality of probes and a plurality of amplicons. In some embodiments, the presence of hybridization indicates DMD AAV construct in a sample. In some embodiments, a method described herein comprises quantifying a level of hybridization between a plurality of probes and a plurality of amplicons. In some embodiments, a quantity associated with a level of hybridization indicates a quantity of DMD AAV construct in the sample.

The present disclosure provides a composition comprising a collection of primers. In some embodiments, a composition comprises a collection of primers and one or more probes. In some embodiments, a collection of primers comprises one or more forward primers. In some embodiments, at least one forward primer has a nucleotide sequence according to SEQ ID NO: 35 or an active fragment thereof.

In some embodiments, a collection of primers comprises one or more reverse primers. In some embodiments, at least one reverse primer has a nucleotide sequence according to SEQ ID NO: 45 or an active fragment thereof.

In some embodiments, at least one probe has a nucleotide sequence according to SEQ ID NO: 55 or an active fragment thereof.

In some embodiments, a composition comprises a collection of primers and one or more probe sequences, wherein at least one primer has a nucleotide sequence according to SEQ ID NO: 35 or an active fragment thereof, at least one primer has a nucleotide sequence according to SEQ ID NO: 45 or an active fragment thereof, and at least one probe has a nucleotide sequence according to SEQ ID NO: 65 or an active fragment thereof.

In some a composition comprises a sample obtained from a subject who has been administered a Hemophilia B (Hem-B) AAV construct. In some embodiments, a Hem-B AAV construct administered to a subject comprises one or more regulatory elements and one or more therapeutic genes of interest. In certain embodiments, a sample obtained from a subject who has been administered a Hem-B AAV construct comprises nucleic acids comprising said Hem-B AAV construct or a fragment thereof. In certain embodiments, a Hem-B AAV construct comprises one or more regulatory elements, wherein the one or more regulatory elements comprise an enhancer, a promoter, a polyA signal sequence, or a combination thereof. In some embodiments, a Hem-B AAV construct comprises a therapeutic gene of interest comprising Factor IX.

In some embodiments, a composition described herein further comprises a plurality of amplicons. In some embodiments, an amplicon (e.g., each amplicon) comprises: (a) a first strand comprising: (i) a nucleotide sequence corresponding to the forward primer, and (ii) a nucleotide sequence corresponding to a portion of a Hem-B AAV construct or fragment thereof; (b) a second strand comprising: (i) a nucleotide sequence of the reverse primer, and (ii) a nucleotide that is complementary to the portion of the Hem-B AAV construct or fragment thereof; or (c) a combination thereof. In some embodiments, the portion of the Hem-B AAV construct or fragment thereof comprises a nucleotide sequence that spans a junction between two regulatory elements, a junction between a construct and regulatory element, or a junction between a regulatory element and the therapeutic gene of interest.

In some embodiments, a composition comprising a plurality of amplicons as described herein also comprises at least one or more probe sequences or active fragments thereof capable of hybridizing with the amplicon. In some embodiments, a composition comprising a plurality of amplicons as described herein also comprises at least one or more probe sequences. In some embodiments, at least one probe sequence has a nucleotide sequence according to SEQ ID NO: 55 or an active fragment and is capable of hybridizing with the amplicon. In some embodiments, one or more probe sequences is amenable to detection. In some embodiments, a level of probe or probe-amplicon hybridization is quantifiable (e.g., a level of fluorescence is quantifiable, a level of fluorescence quenching is quantifiable, a level of total amplicon quantity is quantifiable, or a level of free and/or hybridized probe is quantifiable).

In some embodiments, the present disclosure provides a method comprising contacting a sample obtained from a subject with a composition as described herein, wherein the subject has been administered a Hem-B AAV construct, and wherein the Hem-B AAV construct comprises one or more regulatory elements and a therapeutic gene of interest.

In some embodiments, a method comprises amplifying a target sequence to generate a plurality of amplicons. In some embodiments, a target sequence is a Hem-B AAV construct or fragment thereof. In some embodiments, each amplicon comprises: (i) a first strand comprising: (1) a nucleotide sequence corresponding to the forward primer, and (2) a nucleotide sequence that is complementary to the portion of a Hem-B AAV construct or fragment thereof; or (iii) a combination thereof. In some embodiments, a portion of a Hem-B AAV construct or fragment thereof comprises a nucleotide sequence that spans a junction between two regulatory elements, a junction between a construct and a regulatory element, or a junction between a regulatory element and the therapeutic gene of interest.

In some embodiments of a method described herein, a composition comprises a plurality of probes each with a nucleotide sequence according to SEQ ID NO: 55 or an active fragment thereof. In some embodiments, a composition comprises a plurality of probes and at least one a subset of the plurality of probes comprises a nucleotide sequence according to SEQ ID NO: 55 or an active fragment thereof.

In some embodiments, a method described herein comprises detecting a level of hybridization between a plurality of probes and a plurality of amplicons. In some embodiments, the presence of hybridization indicates Hem-B AAV construct in a sample. In some embodiments, a method described herein comprises quantifying a level of hybridization between a plurality of probes and a plurality of amplicons. In some embodiments, a quantity associated with a level of hybridization indicates a quantity of Hem-B AAV construct in the sample.

In some embodiments, the present disclosure provides a composition comprising a collection of primers and/or collection of primers and probes, wherein one or more forward primers, one or more reverse primers, one or more probes, or a combination thereof is DNA. In some embodiments, the present disclosure provides a composition comprising a collection of primers and/or collection of primers and probes, wherein one or more forward primers, one or more reverse primers, one or more probes, or a combination thereof is DNA that is unmodified, modified, synthetic, comprising an amino modifier, comprising a binding modifier, comprising a spacer, comprising an analog, comprising an intercalation agent, comprising an antisense portion, or any combination thereof. In some embodiments, the present disclosure provides a composition comprising a collection of primers and/or collection of primers and probes, wherein one or more forward primers, one or more reverse primers, one or more probes, or a combination thereof is RNA. In some embodiments, the present disclosure provides a composition comprising a collection of primers and/or collection of primers and probes, wherein one or more forward primers, one or more reverse primers, one or more probes, or a combination thereof is RNA that is unmodified, modified, synthetic, comprising an amino modifier, comprising a binding modifier, comprising a spacer, comprising an analog, comprising an intercalation agent, comprising an antisense portion, or any combination thereof.

In some embodiments, the present disclosure provides a composition comprising a collection of primers and/or collection of primers and probes, wherein one or more forward primers, one or more reverse primers, one or more probes, or a combination thereof comprises a detectable label. In some embodiments wherein a composition comprises one or more detectable labels, one or more detectable labels may not comprise nucleotides. In some embodiments a composition comprises one or more detectable labels that are fluorescent moieties. In some embodiments wherein a composition comprises a detectable label, one or more probes comprise said detectable label. In some embodiments, wherein a composition comprises a probe comprising a detectable label, said detectable label comprises a fluorescent moiety. In some embodiments, wherein a composition comprises more than one primers and/or probes comprising a detectable label that is a fluorescent moiety, said primers and/or probes may comprise the same fluorescent moiety. In some embodiments, wherein a composition comprises more than one more than one primers and/or probes comprising a detectable label that is a fluorescent moiety, said probes may comprise different fluorescent moieties. In some embodiments, wherein a composition comprises one or more probes comprising a detectable label that is a fluorescent moiety, said probe may further comprise one or more quenchers. In some embodiments, wherein a composition comprises one or more probes comprising a detectable label that is a fluorescent moiety, said probe may further comprise more than one quencher molecules that may be the same molecule, or may be different molecules.

In some embodiments, the present disclosure provides a composition in which an active fragment is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 21 nucleotides in length, at least 22 nucleotides in length, at least 23 nucleotides in length, at least 24 nucleotides in length, at least 25 nucleotides in length, at least 26 nucleotides in length, at least 27 nucleotides in length, at least 28 nucleotides in length, or at least 29 nucleotides in length.

In some embodiments, the present disclosure provides a method that includes contacting a sample obtained from a subject with a composition comprising a first primer and a second primer, wherein the subject has been previously administered an AAV construct comprising one or more regulatory elements and a gene of interest, wherein the first primer comprises a sequence corresponding to or complementary to the gene of interest and the second primer comprises a sequence corresponding to or complementary to a regulatory element.

In some embodiments, the present disclosure provides a method that includes (a) contacting a sample obtained from a subject with a composition comprising a first primer and a second primer, wherein the subject has been previously administered an AAV construct comprising one or more regulatory elements and a gene of interest, wherein the first primer comprises a sequence corresponding to or complementary to the gene of interest and the second primer comprises a sequence corresponding to or complementary to a regulatory element; and (b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof (ii) a second strand comprising: (1) a nucleotide sequence of the reverse primer, and (2) a nucleotide sequence that is complementary to the portion of the AAV construct or fragment thereof; or (iii) a combination thereof.

In some embodiments, the present disclosure provides a method that includes (a) contacting a sample obtained from a subject with a composition comprising a first primer and a second primer, wherein the subject has been previously administered an AAV construct comprising one or more regulatory elements and a gene of interest, wherein the first primer comprises a sequence corresponding to or complementary to the gene of interest and the second primer comprises a sequence corresponding to or complementary to a regulatory element; and b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof (ii) a second strand comprising: (1) a nucleotide sequence of the reverse primer, and (2) a nucleotide sequence that is complementary to the portion of the AAV construct or fragment thereof; or (iii) a combination thereof; and wherein the portion of the AAV construct or fragment thereof comprises a nucleotide sequence that spans a junction between a regulatory element and the therapeutic gene of interest.

In some embodiments, the present disclosure provides a method that includes contacting a sample obtained from a subject with a composition comprising a first primer and a second primer, wherein the subject has been previously administered an AAV construct comprising two or more regulatory elements and a gene of interest, wherein the first primer comprises a sequence corresponding to or complementary to a first regulatory element and the second primer comprises a sequence corresponding to or complementary to a second regulatory element.

In some embodiments, the present disclosure provides a method that includes (a) contacting a sample obtained from a subject with a composition comprising a first primer and a second primer, wherein the subject has been previously administered an AAV construct comprising two or more regulatory elements and a gene of interest, wherein the first primer comprises a sequence corresponding to or complementary to a first regulatory element and the second primer comprises a sequence corresponding to or complementary to a second regulatory element; and (b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof; (ii) a second strand comprising: (1) a nucleotide sequence of the reverse primer, and (2) a nucleotide sequence that is complementary to the portion of the AAV construct or fragment thereof; or (iii) a combination thereof.

In some embodiments, the present disclosure provides a method that includes (a) contacting a sample obtained from a subject with a composition comprising a first primer and a second primer, wherein the subject has been previously administered an AAV construct comprising two or more regulatory elements and a gene of interest, wherein the first primer comprises a sequence corresponding to or complementary to a first regulatory element and the second primer comprises a sequence corresponding to or complementary to a second regulatory element; and (b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof (ii) a second strand comprising: (1) a nucleotide sequence of the reverse primer, and (2) a nucleotide sequence that is complementary to the portion of the AAV construct or fragment thereof; or (iii) a combination thereof; and wherein the portion of the AAV construct or fragment thereof comprises a nucleotide sequence that spans a junction between the first regulatory element and the second regulatory element.

In some embodiments, the present disclosure provides a method that includes contacting a sample obtained from a subject with a composition comprising a first primer and a second primer, wherein the subject has been previously administered an AAV construct comprising a construct sequence, one or more regulatory elements and a gene of interest, wherein the first primer comprises a sequence corresponding to or complementary to a construct sequence and the second primer comprises a sequence corresponding to or complementary to a regulatory element.

In some embodiments, the present disclosure provides a method that includes (a) contacting a sample obtained from a subject with a composition comprising a first primer and a second primer, wherein the subject has been previously administered an AAV construct comprising a construct sequence, one or more regulatory elements and a gene of interest, wherein the first primer comprises a sequence corresponding to or complementary to a construct sequence and the second primer comprises a sequence corresponding to or complementary to a regulatory element; and (b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof; (ii) a second strand comprising: (1) a nucleotide sequence of the reverse primer, and (2) a nucleotide sequence that is complementary to the portion of the AAV construct or fragment thereof; or (iii) a combination thereof.

In some embodiments, the present disclosure provides a method that includes (a) contacting a sample obtained from a subject with a composition comprising a first primer and a second primer, wherein the subject has been previously administered an AAV construct comprising a construct sequence, one or more regulatory elements and a gene of interest, wherein the first primer comprises a sequence corresponding to or complementary to a construct sequence and the second primer comprises a sequence corresponding to or complementary to a regulatory element; and (b) performing a polymerase chain reaction to generate a plurality of amplicons, wherein the target sequence is the AAV construct or fragment thereof and each amplicon comprises: (i) a first strand comprising: (1) a nucleotide sequence corresponding to the forward primer, and (2) a nucleotide sequence corresponding to a portion of the AAV construct or fragment thereof; (ii) a second strand comprising: (1) a nucleotide sequence of the reverse primer, and (2) a nucleotide sequence that is complementary to the portion of the AAV construct or fragment thereof; or (iii) a combination thereof and wherein the portion of the AAV construct or fragment thereof comprises a nucleotide sequence that spans a junction between a construct sequence and a regulatory element.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, T, and U, which denote adenosine, cytidine, guanosine, thymidine, and uridine respectively), such nucleotides or peptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. Local administration can involve delivery to cochlear perilymph via, e.g., injection through a round-window membrane or into scala-tympani, a scala-media injection through endolymph, perilymph and/or endolymph following canalostomy. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Amplicon: As used herein, the term “amplicon” refers to an polynucleotide produced through an amplification (e.g., polymerase chain reaction (PCR)) or replication process. An amplicon may also be referred to as a “PCR product.” An amplicon may be specific to a particular polynucleotide construct, oligonucleotide primer pair, and/or oligonucleotide probe. For example, an “amplicon” can refer to (a) a first strand comprising (i) a nucleotide sequence corresponding to the forward primer and (ii) a nucleotide sequence corresponding to a portion of a strand of a construct or fragment thereof, and/or (b) a second strand comprising: (i) a nucleotide sequence of the reverse primer, and (ii) a nucleotide sequence that is complementary to a portion of a strand of a construct or fragment thereof. In some embodiments, an “amplicon” can refer to (a) a first strand comprising (i) a nucleotide sequence corresponding to the forward primer, (ii) a nucleotide sequence corresponding to a portion of a strand of a construct or fragment thereof, and (iii) a nucleotide sequence complementary to the reverse primer; and/or (b) a second strand comprising: (i) a nucleotide sequence of the reverse primer, (ii) a nucleotide sequence that is complementary to a portion of a strand of a construct or fragment thereof, and (iii) a nucleotide sequence complementary to the forward primer.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Cleavage: As used herein, the term “cleavage” refers to generation of a break in DNA. For example, in some embodiments, cleavage could refer to either a single-stranded break or a double-stranded break depending on a type of nuclease that may be employed to cause such a break.

Combination Therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For Amino | | |
|---|---|---|
| Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, |
| | | D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, He, D-Ile, Orn, D-Orn |
| Methionine | M | D-Met, S-Me-Cys, Ile, D-Ile, Leu, D-Leu, Val, D- |
| | | Val |
| Phenylalanine | F | D-Phe, Tyr, D-Thr, L-Dopa, His, D-His, Trp, D- |
| | | Trp, Trans-3,4 or 5-phenylproline, AdaA, AdaG, |
| | | cis-3,4 or 5-phenylproline, Bpa, D-Bpa |
| Proline | P | D-Pro, L-I-thioazolidine-4-carboxylic acid, D-or- |
| | | L-1-oxazolidine-4-carboxylic acid (Kauer, U.S. |
| | | Pat. No. (4,511,390) |
| Serine | S | D-Ser, Thr, D-Thr, allo-Thr, Met, D-Met, Met (O), |
| | | D-Met (O), L-Cys, D-Cys |
| Threonine | T | D-Thr, Ser, D-Ser, allo-Thr, Met, D-Met, Met (O), |
| | | D-Met (O), Val, D-Val |
| Tyrosine | Y | D-Tyr, Phe, D-Phe, L-Dopa, His, D-His |
| Valine | V | D-Val, Leu, D-Leu, He, D-Ile, Met, D-Met, AdaA, |
| | | AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Editing: As used herein, the term “edit,” “editing,” or “edited” refers to a method of altering a nucleic acid sequence of a polynucleotide (e.g., a wild type naturally occurring nucleic acid sequence or a mutated naturally occurring sequence) by selective deletion of a specific nucleic acid sequence (e.g., a genomic target sequence), a given specific inclusion of new sequence through use of an exogenous nucleic acid sequence, or a replacement of nucleic acid sequence with an exogenous nucleic acid sequence. In some embodiments, such a specific genomic target includes, but may be not limited to, a chromosomal region, mitochondrial DNA, a gene, a promoter, an open reading frame or any nucleic acid sequence.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Gene Therapy: As used herein, the term “gene therapy” means a therapy that involves administering to a subject a biologically or synthetically produced composition which (a) delivers, in vivo or ex vivo, nucleic acid, such as DNA or RNA, and results in the transient or stable expression of an RNA sequence and/or protein that is encoded by the delivered DNA or RNA, which expression may be either from an episome or from the nucleic acid having integrated into the genome, (b) delivers an mRNA and results in the expression of a protein from the delivered mRNA, or (c) delivers any gene-editing or base-editing system, including but not limited to TALENS, Megatal, zinc-finger proteins, CRISPR/Cas9 or other Cas-based systems, such delivery of a gene-editing or base-editing system resulting in (1) introduction of an epigenetic modification to a chromosomal sequence element, which epigenetic modification alters transcription of a gene associated with the chromosomal sequence element, or (2) deletion or insertion or modification of DNA or RNA sequence(s) of a genome or transcriptome.

Genome Editing System: As used herein, the term “genome editing system” refers to any system having DNA editing activity. Among other things, DNA editing activity can include deleting, replacing, or inserting a DNA sequence in a genome. In some embodiments, a genome editing system comprises RNA-guided DNA editing activity. In some embodiments, a genome editing system of the present disclosure includes more than one component. In some embodiments, a genome editing system includes at least two components adapted from naturally occurring CRISPR systems: a guide RNA (gRNA) and an RNA-guided nuclease. In certain embodiments, these two components form a complex that is capable of associating with a specific nucleic acid sequence and editing DNA in or around that nucleic acid sequence, for instance by making one or more of a single-strand break (an SSB or nick), a double-strand break (a DSB) and/or a point mutation. In some embodiments, genome editing systems of the present disclosure lack a component having cleavage activity but maintain a component(s) having DNA binding activity. In some such embodiments, a genome editing system of the present disclosure comprises a component(s) that functions as an inhibitor of DNA activity, e.g., transcription, translation, etc. In some embodiments, a genome editing system of the present disclosure comprises a component(s) fused to modulators to modulate target DNA expression.

Genomic modification: As used herein, the term “genomic modification” refers to a change made in a genomic region of a cell that permanently alters a genome (e.g., an endogenous genome) of that cell. In some embodiments, such changes are in vitro, ex vivo, or in vivo. In some embodiments, every cell in a living organism is modified. In some embodiments, only a particular set of cells such as, e.g., in a specific organ, is modified. For example, in some embodiments, a genome is modified by deletion, substitution, or addition of one or more nucleotides from one or more genomic regions. In some embodiments, a genomic modification is performed in a stem cell or undifferentiated cell. In some such embodiments, progeny of a genomically modified cell or organism will also be genomically modified, relative to a parental genome prior to modification. In some embodiments, a genomic modification is performed on a mature or post-mitotic cell such that no progeny will be generated and thus, no genomic modifications propagated other than in a particular cell.

Heterologous: As used herein, the term “heterologous” may be used in reference to one or more regions of a particular molecule as compared to another region and/or another molecule. For example, in some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Modulating: As used herein, the term “modulating,” means mediating a detectable increase or decrease in a level of a response in a subject compared with a level of a response in a subject in absence of a treatment or compound, and/or compared with a level of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Target site: As used herein, the term “target site” means a portion of a nucleic acid to which a binding molecule, e.g., a microRNA, an siRNA, a guide RNA (“gRNA”) or a guide RNA:Cas complex, will bind, provided sufficient conditions for binding exist. In some embodiments, a nucleic acid comprising a target site is double stranded. In some embodiments, a nucleic acid comprising a target site is single stranded. Typically, a target site comprises a nucleic acid sequence to which a binding molecule, e.g., a gRNA or a gRNA:Cas complex described herein, binds and/or that is cleaved as a result of such binding. In some embodiments, a target site comprises a nucleic acid sequence (also referred to herein as a target sequence or protospacer) that is complementary to a DNA sequence to which the targeting sequence (also referred to herein as the spacer) of a gRNA described herein binds. In some embodiments in the context of RNA-guided nucleases, e.g., CRISPR/Cas nucleases, a target site typically comprises a nucleotide sequence (also referred to herein as a target sequence or a protospacer) that is complementary to a sequence comprised in a gRNA (also referred to herein as the targeting sequence or the spacer) of an RNA-programmable nuclease. In some such embodiments, a target site further comprises a protospacer adjacent motif (PAM) at the 3′ end or 5′ end adjacent to the gRNA-complementary sequence. For an RNA-guided nuclease Cas9, a target sequence may be, in some embodiments, 16-24 base pairs plus a 3-6 base pair PAM (e.g., NNN, wherein N represents any nucleotide). Exemplary PAM sequences for RNA-guided nucleases, such as Cas9, are known to those of skill in the art and include, without limitation, NNG, NGN, NAG, NGA, NGG, NGAG and NGCG wherein N represents any nucleotide. In addition, Cas9 nucleases from different species have been described, e.g., S. thermophilus recognizes a PAM that comprises the sequence NGGNG, and Cas9 from S. aureus recognizes a PAM that comprises the sequence NNGRRT. In some embodiments, Cas9 from S. aureus recognizes a PAM that comprises the sequence NNNRRT. Additional PAM sequences are known in the art, including, but not limited to NNAGAAW and NAAR (see, e.g., Esvelt and Wang, Molecular Systems Biology, 9:641 (2013), the entire content of which is incorporated herein by reference). For example, the target site of an RNA-guided nuclease, such as, e.g., Cas9, may comprise a structure [Nz]-[PAM], where each N is, independently, any nucleotide, and z is an integer between 1 and 50. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, Z is 20.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome (e.g., increased electrical activity resulting in chronic depolarization that leads to cell death). Such a variant is referred to herein as a loss-of-function variant. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type(e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide.

### BRIEF DESCRIPTION OF THE DRAWING

FIG. 1—depicts a schematic representation of an exemplary qPCR assay primer design. The schematic displays different regions where primer and probes can be used in a qPCR assay. Primer and probe combinations were designed to be specific for the virus and gene of interest. Regions in the recombinant viral genomes (for the different assays) that span target gene-specific sequences that do not exist in this order in the human genome or AAV genome were selected. For each assay, multiple primer and probes were designed for each region and screened for the most robust and specific combination.

FIG. 2—depicts a calibration curve plot of an exemplary DMD AAV construct in Siemens Storage Buffer 2 (SSB2) for value assignments, as described in Example 1.

FIG. 3—depicts the results of precision calculations for quantifying an exemplary DMD AAV construct in saliva, stool, urine, and whole blood biological samples, as described in Example 1.

FIG. 4—depicts the stability of an exemplary DMD AAV construct over 48 hours at ambient temperature in saliva, stool, urine, and whole blood biological samples.

FIG. 5—depicts the long-term stability of an exemplary DMD AAV construct in saliva, stool, urine, and whole blood biological samples stored over 9 months at−80° C.

FIG. 6—depicts the stability of an exemplary DMD AAV construct in saliva, stool, urine, and whole blood biological samples over multiple freeze/thaw cycles.

FIG. 7—depicts the effects of MNase treatment on the sample recovery of an exemplary DMD AAV construct in saliva, urine and stool samples.

FIG. 8—depicts the effects of MNase and DNase treatment on the sample recovery of an exemplary DMD AAV construct in whole blood samples.

FIG. 9—depicts the linearity and accuracy of exemplary quantification assays for measuring an exemplary Hem-B AAV construct in plasma (panel 9A), PBMC (panel 9B), saliva (panel 9C), semen (panel 9D), stool (panel 9E), or urine (panel 9F).

FIG. 10—depicts the variability of exemplary quantification assays for measuring an exemplary Hem-B AAV construct in plasma (panel 10A), PBMC (panel 10B), saliva (panel 10C), semen (panel 10D), stool (panel 10E), or urine (panel 10F) from five unique donors.

FIG. 11—depicts the effects of MNase treatment on recovery of an exemplary Hem-B AAV construct sample, double stranded DNA, or single stranded DNA from saliva (panel 11A), semen (panel 11B), stool (panel 11C), or urine (panel 11D) samples.

FIG. 12—depicts the effects of MNase treatment on recovery of an exemplary Hem-B AAV construct sample from saliva, semen, stool, or urine samples.

FIG. 13—depicts the stability of an exemplary Hem-B AAV construct over 48 hours at ambient temperature in whole blood samples from three unique donors.

FIG. 14—depicts the stability of an exemplary Hem-B AAV construct over 48 hours at ambient temperature in plasma (panel 14A), PBMC (panel 14B), saliva (panel 14C), semen (panel 14D), stool (panel 14E), or urine (panel 14F) samples.

FIG. 15—depicts the stability of an exemplary Hem-B AAV construct over multiple freeze/thaw cycles in plasma (panel 15A), PBMC (panel 15B), saliva (panel 15C), semen (panel 15D), stool (panel 15E), or urine (panel 15F) samples.

FIG. 16—depicts the long-term stability of an exemplary Hem-B AAV construct stored at −80° C. in plasma (panel 16A), PBMC (panel 16B), saliva (panel 16C), semen (panel 16D), stool (panel 16E), or urine (panel 16F) samples.

FIG. 17—depicts a series of quantitative PCR amplification plots for quantification of an exemplary DMD AAV construct. Targeting Region 1 of an exemplary DMD AAV construct are primer and probe combination DMD-FWD-B (SEQ ID NO: 3), DMD-REV-A (SEQ ID NO: 9), and DMD-PROBE-A (SEQ ID NO: 17) (panel 17A). Targeting Region 2 of an exemplary DMD AAV construct are primer and probe combination DMD-FWD-E (SEQ ID NO: 63), DMD-REV-E (SEQ ID NO: 67), and DMD-PROBE-F (SEQ ID NO: 73) (panel 17B).

FIG. 18—depicts a series of quantitative PCR amplification plots for quantification of an exemplary DMD AAV construct and quantification of an internal control (IC). Targeting Region 3 of an exemplary DMD AAV construct are primer and probe combination DMD-FWD-F (SEQ ID NO: 69), DMD-REV-G (SEQ ID NO: 71), and DMD-PROBE-G (SEQ ID NO: 77) (panel 18A). Targeting an internal control DNA are primers and probes comprising Cy5 fluorescence (panel 18B).

FIG. 19—depicts a series of results for quantitative PCR amplification characteristics such as maximum fluorescence, slope, and earliest Ct for primer and probe combinations targeting DMD multiplexed with a primer and probe combination targeting an internal control DNA pool (panel 19A). Set 1 represents a combination of DMD-FWD-A (SEQ ID NO: 1), DMD-REV-B (SEQ ID NO: 11), and DMD-PROBE-A (SEQ ID NO: 17). Set 2 represents a combination of DMD-FWD-A (SEQ ID NO: 1), DMD-REV-B (SEQ ID NO: 11), and DMD-PROBE-B (SEQ ID NO: 21). Set 3 represents a combination of DMD-FWD-B (SEQ ID NO:3), DMD-REV-A (SEQ ID NO: 9), and DMD-PROBE-A (SEQ ID NO: 17). Set 4 represents a combination of DMD-FWD-C(SEQ ID NO: 5), DMD-REV-D (SEQ ID NO: 15), and DMD-PROBE-D (SEQ ID NO: 29). Set 5 represents a combination of DMD-FWD-D (SEQ ID NO: 7), DMD-REV-C(SEQ ID NO: 13), and DMD-PROBE-C (SEQ ID NO: 25). In addition, results are shown for multiple concentrations of primers and probes for DMD specific oligonucleotides (panel 19A) and IC specific oligonucleotides (panel 19B).

FIG. 20—depicts a series of quantitative PCR amplification plots for quantification of an exemplary Hem-B AAV construct. Targeting Region 1 of an exemplary DMD AAV construct are primer and probe combination HemB-FWD-A (SEQ ID NO: 33), HemB-REV-A (SEQ ID NO: 39), and HemB-PROBE-A (SEQ ID NO: 47) (panel 20A). Targeting Region 2 of an exemplary Hem-B AAV construct are primer and probe combination HemB-FWD-B (SEQ ID NO:35), HemB-REV-D (SEQ ID NO: 45), and HemB-PROBE-C(SEQ ID NO: 55) (panel 20B). Targeting Region 3 of an exemplary Hem-B AAV construct are primer and probe combination HemB-FWD-D (SEQ ID NO: 81), HemB-REV-F (SEQ ID NO: 87), and HemB-PROBE-E (SEQ ID NO:89) (panel 20C).

FIG. 21—depicts a series of results for quantitative PCR amplification characteristics such as maximum fluorescence, slope, and earliest Ct for primer and probe combinations targeting exemplary Hem-B AAV construct. Set 1 represents a combination of HemB-FWD-B (SEQ ID NO: 35), HemB-REV-C(SEQ ID NO: 43), and HemB-PROBE-B (SEQ ID NO: 51), at 150 nM final concentration for each oligonucleotide. Set 2 represents a combination of HemB-FWD-B (SEQ ID NO: 35), HemB-REV-C(SEQ ID NO: (43), and HemB-PROBE-B (SEQ ID NO: (51) at 200 nM final concentration for each oligonucleotide. Set 3 represents a combination of HemB-FWD-C(SEQ ID NO: 37), HemB-REV-B (SEQ ID NO: 41), and HemB-PROBE-D (SEQ ID NO: 59) at 250 nM final concentration for each oligonucleotide. Set 4 represents a combination of HemB-FWD-C(SEQ ID NO: 37), HemB-REV-B (SEQ ID NO: 41), and HemB-PROBE-D (SEQ ID NO: 59), at 300 nM final concentration for each oligonucleotide. Set 5 represents a combination of HemB-FWD-B (SEQ ID NO: 35) HemB-REV-D (SEQ ID NO: 45), and HemB-PROBE-C(SEQ ID NO: 55), at 100 nM final concentration for each oligonucleotide. Set 6 represents a combination of HemB-FWD-B (SEQ ID NO: 35), HemB-REV-D (SEQ ID NO: 45), and HemB-PROBE-C (SEQ ID NO: 55), at 150 nM final concentration for each oligonucleotide. Set 7 represents a combination of HemB-FWD-E (SEQ ID NO: 83), HemB-REV-F (SEQ ID NO: 85), and HemB-PROBE-E (SEQ ID NO: 89), at 200 nM final concentration for each oligonucleotide. Set 8 represents a combination of HemB-FWD-D (SEQ ID NO: 81), HemB-REV-F (SEQ ID NO: 87), and HemB-PROBE-E (SEQ ID NO: 89), at 200 nM final concentration for each oligonucleotide. Set 9 represents a combination of HemB-FWD-D (SEQ ID NO: 81), HemB-REV-F (SEQ ID NO: 87), and HemB-PROBE-E (SEQ ID NO: 89), at 300 nM final concentration for each oligonucleotide. Set 10 represents a combination of HemB-FWD-A (SEQ ID NO: 33), HemB-REV-A (SEQ ID NO: 39), and HemB-PROBE-A (SEQ ID NO: 47), at 150 nM final concentration for each oligonucleotide. Sets 1-6 are targeting region 2 of the exemplary Hem-B AAV constructs, sets 7-9 are targeting region 3 of the exemplary Hem-B AAV constructs, set 10 is targeting region 1 of the exemplary Hem-B AAV constructs.

FIG. 22—depicts a series of quantitative PCR amplification plots for quantification of an exemplary Hem-B AAV construct using primer probe combination set 10 (panel 22A), set 6 (panel 22B), and set 9 (panel 22C).

FIG. 23—depicts an exemplary set of quantitative PCR amplification results for multiplexed Exemplary Hem-B AAV construct amplification and exemplary Internal Control amplification across six tissue types: feces, plasma, PMBC, saliva, semen, and urine

### DETAILED DESCRIPTION OF CERTAIN EMBODIMENTS

### Gene Therapy

Gene therapy refers to techniques that involve the introduction of gene(s) or modification of gene expression to treat, ameliorate, and/or prevent disease. There are several approaches to gene therapy, including but not limited to: replacing a mutated gene that causes disease with a healthy copy of the gene; inactivating, or “knocking out,” a mutated gene that is functioning improperly; and/or introducing a gene into the body to help fight a disease. Gene therapy is a promising treatment option for a number of diseases (including inherited disorders, some types of cancer, and certain viral infections). Generally, gene therapy is tested for diseases that have a known genetic association and/or for diseases where the current standard of care is considered lacking.

Clinical trials involving gene therapy techniques are subject to regulations, which can require certain monitoring guidelines are met. One such regulation involving viral delivery methods requires an accurate and often continual measuring of viral dosage and viral “shedding” (the viral particles released through any mechanism from the test subject). The present disclosure provides compositions and methods that aid in the essential monitoring of viral shedding, providing physicians and scientists with robust and reliable measurements.

### Exemplary Conditions

Duchene Muscular Dystrophy

Duchenne muscular dystrophy (DMD) is an X-linked recessive disorder that affects approximately 1 in 5000 live male births (Mendell et al., 2012; and Moat et al., 2013; both of which are incorporated herein by reference for any purpose). It was first described in detail in the 1860s by the French neurologist Guillaume-Benjamin-Amand Duchenne (Duchenne 1861, which is incorporated herein by reference for any purpose). Patients with Duchenne muscular dystrophy usually exhibit motor symptoms within the first 3 years of life. Most commonly, they may have a “waddling” gait that results from hip-girdle weakness and require the use of their hands when they get up from the floor (Gower's maneuver).

The disease is due to an absence of the dystrophin protein in the skeletal muscle membrane, and muscles lacking dystrophin are more susceptible to mechanical injury. Absence of dystrophin may be demonstrated by the absence of immunostaining for dystrophin on muscle biopsy. Genetic testing, however, has become more readily available in recent years and has become the standard method of diagnostic confirmation. Typically, genetic testing starts with screening for duplications or deletions either by multiplex ligation-dependent probe amplification (MLPA) or by microarray analysis. If duplication/deletion testing is negative, then sequencing of all 79 exons is performed to detect missense, nonsense, splice site, and small indel mutations (Birnkrant et al., part 3, 2018; which is incorporated herein by reference for any purpose). Dependent upon the genetic testing results, various gene therapy avenues may be available.

The DMD gene is considered one of the largest genes in the human genome, and many disruptive mutations have been reported. De novo mutations appear to be common, with estimates ranging between 12 and 33% of patients with DMD (Shieh, 2018; which is incorporated herein by reference for any purpose). Estimates of the prevalence of different mutation types vary, but reports suggest that, among DMD patients, 69% have large deletions, 11% have large duplications, 10% have nonsense mutations, 7% have missense or small indels, and another 3% have intronic or other mutations (Shieh, 2018; which is incorporated herein by reference for any purpose). The large size of the dystrophin protein means that full gene replacement therapy for DMD is only feasible with large viruses such as adenoviral vectors. Alternatively, rather than introducing the entirety of the large DMD gene, miniaturized but efficacious versions of dystrophin, often nicknamed “minidystrophins” or “microdystrophins”, can be employed. These mini-genes are often small enough to be packaged in an adeno-associated virus (AAV). Over the past 16 years, the preclinical and clinical investigational experience with AAVs has grown to provide a better understanding of the safety profile of these viruses. This experience and knowledge base has made AAV vector delivery of gene therapies a key clinical candidate, including for the treatment of muscular dystrophy through the delivery of micro/minidystrophin genes. While AAV based gene therapy may be a breakthrough, there remains a pressing need for accurate and reproducible materials and methods for monitoring AAV shedding in patients being treated for DMD using gene therapy.

Hemophilia B

Hemophilia B, also called factor IX (FIX) deficiency or Christmas disease, is a genetic disorder caused by missing or defective Factor IX, a clotting protein. Although it is passed down from parents to children, about ⅓ of cases are caused by de-novo mutations. According to the US Centers for Disease Control and Prevention, hemophilia occurs in approximately 1 in 5,000 live births. There are about 20,000 people with hemophilia in the US. All races and ethnic groups are affected. Hemophilia B is four times less common than hemophilia A. People with hemophilia B bleed longer than other people. Bleeds can occur internally, into joints and muscles, or externally, from minor cuts, dental procedures or trauma. How frequently a person bleeds and how serious the bleeds are depends on how much FIX is in the plasma, Hemophilia B is tested for using assays that evaluate clotting time. Additionally, genetic testing is utilized to determine the underlying molecular mechanism of an individual's hemophilia B, as there are over 1100 unique mutations known to cause hemophilia B.

The main medication to treat hemophilia B is concentrated FIX product, called clotting factor or simply factor. Recombinant factor products, which are developed in a lab through the use of DNA technology preclude the use of human-derived pools of donor-sourced plasma. While plasma-derived FIX products are still available, approximately 75% of the hemophilia community takes a recombinant FIX product. These factor therapies are infused intravenously through a vein in the arm or a port in the chest, and often patients require routine costly, time consuming, and burdensome treatment. Gene therapy is proving to be amenable and potentially highly efficacious for the treatment of hemophilia B. However while AAV based gene therapy may be a breakthrough, there remains a pressing need for accurate and reproducible materials and methods for monitoring AAV shedding in patients being treated for hemophilia B using gene therapy.

### Viral Vectors for Therapeutic Uses

Among other things, the present disclosure provides compositions and methods for measuring polynucleotides. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide sequence of interest or characteristic portion thereof.

Those of skill in the art will be capable of selecting suitable constructs for measurement. In some embodiments, a construct is a viral construct. In some embodiments, a viral construct is a lentivirus, retrovirus, adenovirus, or adeno-associated virus construct. In some embodiments, a construct is an adeno-associated virus (AAV) construct (see, e.g., Asokan et al., Mol. Ther. 20: 699-7080, 2012, which is incorporated in its entirety herein by reference). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs suitable for measurement may be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

AAV for Therapeutic Uses

Previous research has identified Adeno Associate Virus (AAV) as a suitable vector for the delivery of therapeutic oligonucleotides to subjects in need thereof.

Gene therapy is evolving rapidly for the treatment of genetic disorders, and for many patients, the success of these therapeutics is their only hope for a cure (Angeula & High 2019; and Carlton 2018). During gene therapy, a healthy copy of the gene causing the disease is delivered to the patient via a viral vector (Angeula & High, 2019; and Carlton, 2018). Many regulatory bodies (EMA, FDA, etc.) require monitoring of the shed virus as part of the gene therapy clinical trial (FDA, 2015; ICH, 2009; GTWP, BWP, and SWP, 2008; and Bubela et al., 2019). Detection of the virus in bodily fluids might be critical to understand environmental consequences or potential long-term effects that may lead to an increase of neutralizing antibodies (NAb) against adeno-associated virus in society (Bubela et al., 2019; and Rodrigues et al., 2018).

One of the most common gene therapy vectors is adeno-associated virus (AAV). AAV is used due to its apparent lack of pathogenesis and replication competency (Angeula & High 2019; and Carlton 2018). Patients undergoing recombinant AAV (rAAV) gene therapy treatment are injected with high titers of rAAV. Patients undergoing this kind of treatment clear excess virus through a process called shedding. Shedding refers to the excretion or release of the vector-based gene therapy product from patients' excreta (stool), secreta (urine, saliva, and semen), or blood products (whole blood, plasma, or PBMCs) (FDA, 2015; ICH, 2009; GTWP, BWP, and SWP, 2008; and Bubela et al., 2019). Even though AAV is not pathogenic, shedding raises a possible risk to people encountering patients undergoing gene therapy treatment. The risk of exposure to the virus might result in increase of neutralizing antibodies against AAVs and rAAV-based therapy in society (Bubela et al., 2019; and Rodrigues et al., 2018). Monitoring viral concentration in shed patients' samples is necessary to understand possible modes of vector transmission and is a requirement of multiple regulatory agencies.

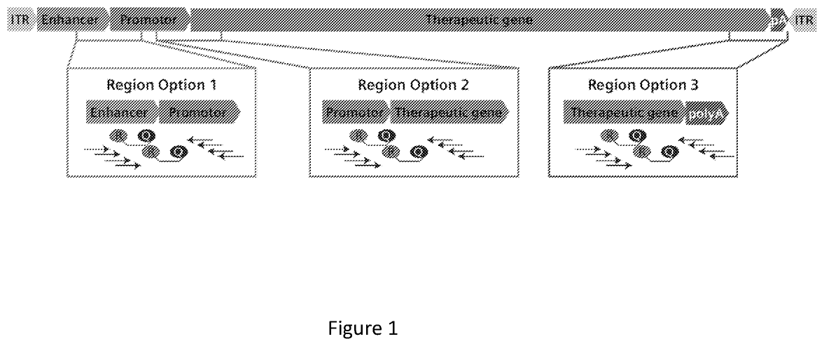

There are two key requirements for developing a shedding assay: The assay must be developed for a diverse range of sample types, and must be quantitative (FDA, 2015). Herein are described novel compositions comprising specific primers to quantify viral DNA in patient samples (FIG. 1). These compositions are suitable for quantitative polymerase chain reaction (qPCR) for quantification of viral DNA in samples (FIG. 1). One challenge is the ability to consistently purify viral DNA from a variety of complex matrices. Therefore, a need exists for materials and methods that can create reproducible, efficient, and scalable extraction and quantification of viral DNA from shed samples.

Described herein are new compositions and protocols for monitoring viral-shed DNA from many different bodily fluids, including semen, saliva, urine, whole blood, PBMC, plasma, and stool. The data presented represents an optimized quantification protocol from a diverse cohort of shedding compartments to establish consistent and reproducible shedding assays.

### Viral Shedding

Existing DNA quantification and/or sequencing materials and methods for accurately detecting, identifying, and quantifying foreign and/or therapeutic oligonucleotides are often found wanting. In particular, current materials and methods for quantification of gene therapy constructs in clinical samples and/or shed from patients often fail to accurately and reproducibly quantify the shed construct and/or genome titers to the levels described by the appropriate regulatory and/or advisory bodies (e.g., the GTWP, BWP, SWP and/or FDA).

In some embodiments, the materials and methods described herein can detect and quantify gene therapy constructs found as unbound and/or free oligonucleotides. In some embodiments, the materials and methods described herein can detect and quantify gene therapy constructs associated with a viral particle. As described herein, there are many suitable gene therapy vectors (e.g., viral particles) that may house gene therapy oligonucleotides amenable to quantification using the materials and methods described herein. In certain embodiments, a gene therapy construct detected and quantified is an oligonucleotide associated with a recombinant adeno-associated virus (rAAV) particle.

Previous assays have not demonstrated sufficient performance to be widely accepted in clinical laboratories and have not been submitted for regulatory approval. Most are used as research assays or local home-brew laboratory developed tests due to these performance limitations. Previous assay designs have not solved these limitations.

The present disclosure, in contrast, results in uniquely high performing assay design prototypes. The top performing design meets IVD commercialization criteria of >95% successful amplification with known global sequence variants. This performance criteria has been previously demonstrated to be indicative of sufficient sensitivity across genetic variants to be implemented for routine clinical use with samples in international studies.

Measuring Viral Associated Oligonucleotide Construct Titers from Biological Sources

In some embodiments, the compositions and methods described herein are useful for detecting and quantifying oligonucleotide constructs delivered by rAAV particle. In some embodiments, the detection and quantification is conducted on samples obtained from a patient and are measurements of viral shedding. In some embodiments, the viral associated oligonucleotide constructs shed from a patient may be direct to a specific gene therapy construct, such as but not limited to constructs for treating: Duchenne Muscular Dystrophy, and/or Hemophilia B.

### Amplification Oligonucleotide Sequences and Primer Sets

In some embodiments, compositions described herein comprise forward and reverse oligonucleotides suitable and specific for amplification and subsequent quantification of known gene therapy constructs. In some embodiments, compositions comprising forward and reverse amplification oligonucleotides may additionally comprise sequence specific probes suitable for real time quantification of amplicon products. In some embodiments, sequence specific probes may be probes designed for amplicon quantification using TaqMan™ quantitative polymerase chain reaction (qPCR) protocols.

In some embodiments, quantification probes and primers may be designed to hybridize with sequences specific to non-naturally occurring construct specific junctions. In some embodiments, junctions may be spanning construct-to-regulatory elements, (e.g., spanning rAAV inverted terminal repeat sequences to promoter and/or 3′ UTR regions). In some embodiments, junctions may be spanning regulatory-to-regulatory elements, (e.g., spanning a promoter to enhancer junction, and/or spanning a 3′ UTR to polyA signal sequence). In some embodiments, junctions may be spanning regulatory-to-payload elements (e.g., spanning promoter and/or 3′ UTR regions to gene therapy specific payload sequences, i.e., Dystrophin, Mini-Dystrophin, and/or Factor IX). In some embodiments, junctions may be spanning construct-to-payload elements (e.g., spanning rAAV inverted terminal repeat sequences to gene therapy specific payload sequences).

In some embodiments, primers and/or probes may be screened to determine the total pool of primers and/or probes amenable to multiplexing. In some embodiments, one or more primers and/or one or more probes may be utilized in a multiplexing assay. In certain embodiments, primers and/or probes are screened for crosstalk and/or undesirable oligonucleotide interactions. In some embodiments, primers and/or probes are tested to evaluate their specificity when additionally exposed to background genomic DNA. In some embodiments, candidate primers and/or probes are further optimized to include additions, truncations, and/or nucleotide modifications to increase assay performance. In certain embodiments, primers and/or probes tested in numerous different concentrations (e.g., 100 nM, 200M, 300 nM, 400 nM, and/or 500 nM) to increase assay performance. In some embodiments, specific primer and/or probe combinations may be utilized to increase assay performance. In some embodiments, any or all of the factors described herein may be utilized in determining an appropriate primer and/or primer and probe set for the accurate amplification and/or quantification of an AAV construct.

In some embodiments, an active fragment of an oligonucleotide described herein is at most the length of a particular primer and/or probe minus one nucleotide. In some embodiments, an active fragment is at most 29 nucleotides, is at most 28 nucleotides, is at most 27 nucleotides, is at most 26 nucleotides, is at most 25 nucleotides, is at most 24 nucleotides, is at most 23 nucleotides, is at most 22 nucleotides, is at most 21 nucleotides, is at most 20 nucleotides, is at most 19 nucleotides, is at most 18 nucleotides, is at most 17 nucleotides, is at most 16 nucleotides, is at most 15 nucleotides, is at most 14 nucleotides, is at most 13 nucleotides, is at most 12 nucleotides, is at most 11 nucleotides, or is at most 10 nucleotides.

In some embodiments, primer combinations and/or primer and probe combinations may be multiplexed with additional primer and/or primer and probe sets. In some embodiments, the sensitivity of a primer combination and/or primer and probe combination may be altered with multiplexing (e.g., if CTs of single assays come up much earlier than in multiplex assay, then multiplexing may have an inhibitory effect and this may alter the overall sensitivity). In some embodiments, the specificity of a primer combination and/or primer and probe combination may be altered with multiplexing (e.g., if in a multiplex assay wherein the primers and/or primer and probe combinations are given multiple target DNA populations as one template, a specific primer combinations and/or primer and probe combinations may not produce signal in the absence of its target DNA population, if signal is present, specificity may be lower than in the absence of the additional DNA population).

Oligonucleotide Sequences for DMD AAV Specific Quantification

Each of the following Primer sequences is provided in 5′ to 3′ order; those skilled in the art will recognize than in certain embodiments a polynucleotide may be an RNA molecule, or a DNA molecule. In certain embodiments, primary screening comprised the evaluation of 37 primer combinations and 9 different probes, culminating in 40 different primer-probe combinations.

DMD-FWD-A
SEQ ID NO: 1
AGA CAG ACA CTC AGG AGC CAG CC
DMD-FWD-A.U
SEQ ID NO: 2
AGA CAG ACA CUC AGG AGC CAG CC
DMD-FWD-B
SEQ ID NO: 3
ACC ACC TCC ACA GCA CAG ACA GA
DMD-FWD-B.U
SEQ ID NO: 4
ACC ACC UCC ACA GCA CAG ACA GA
DMD-FWD-C
SEQ ID NO: 5
CCT ACT ACA TCA ACC ACG AGA CC
DMD-FWD-C.U
SEQ ID NO: 6
CCU ACU ACA UCA ACC ACG AGA CC
DMD-FWD-D
SEQ ID NO: 7
GGA TAA GTA CCG CTA CCT GTT CA
DMD-FWD-D.U
SEQ ID NO: 8
GGA UAA GUA CCG CUA CCU GUU CA
DMD-REV-A
SEQ ID NO: 9
AGC AGT CCT CCA CTT CCT CCC AC
DMD-REV-A.U
SEQ ID NO: 10
AGC AGU CCU CCA CUU CCU CCC AC
DMD-REV-B
SEQ ID NO: 11
TGC ACG TCC TCT CTC TCG TAG CA
DMD-REV-B.U
SEQ ID NO: 12
UGC ACG UCC UCU CUC UCG UAG CA
DMD-REV-C
SEQ ID NO: 13
CTA GGG ATC TGG ATG CTA TCG TG
DMD-REV-C.U
SEQ ID NO: 14
CUA GGG AUC UGG AUG CUA UCG UG
DMD-REV-D
SEQ ID NO: 15
CTC TGA TAC AGC TCG GTC ATC TT
DMD-REV-D.U
SEQ ID NO: 16
CUC UGA UAC AGC UCG GUC AUC UU
DMD-PROBE-A
SEQ ID NO: 17
/56-FAM/AGC GTC GAG /ZEN/CGG CCG
ATC CGC CAC C/3IABkFQ/
DMD-PROBE-A.U
SEQ ID NO: 18
/56-FAM/AGC GUC GAG /ZEN/CGG CCG
AUC CGC CAC C/3IABkFQ/
DMD-PROBE-A.0
SEQ ID NO: 19
AGC GTC GAG CGG CCG ATC CGC CAC C
DMD-PROBE-A.0.U
SEQ ID NO: 20
AGC GUC GAG CGG CCG AUC CGC CAC C
DMD-PROBE-B
SEQ ID NO: 21
/56-FAM/CAC CAA AGC /ZEN/ ATG
GTG GCG GAT CG /3IABkFQ/
DMD-PROBE-B.U
SEQ ID NO: 22
/56-FAM/CAC CAA AGC /ZEN/ AUG
GUG GCG GAU CG /3IABkFQ/
DMD-PROBE-B.0
SEQ ID NO: 23
CAC CAA AGC ATG GTG GCG GAT CG
DMD-PROBE-B.0.U
SEQ ID NO: 24
CAC CAA AGC AUG GUG GCG GAU CG
DMD-PROBE-C
SEQ ID NO: 25
/56-FAM/ATC AGA GGA /ZEN/GAC TGG
GCC TGC TGC T /3IABkFQ/
DMD-PROBE-C.U
SEQ ID NO: 26
/56-FAM/AUC AGA GGA /ZEN/GAC UGG
GCC UGC UGC U /3IABkFQ/
DMD-PROBE-C.0
SEQ ID NO: 27
ATC AGA GGA GAC TGG GCC TGC TGC T
DMD-PROBE-C.0.U
SEQ ID NO: 28
AUC AGA GGA GAC UGG GCC UGC UGC U
DMD-PROBE-D
SEQ ID NO: 29
/56-FAM/CAG ACC ACC /ZEN/ TGC
TGG GAC CAC CCT /3IABkFQ/
DMD-PROBE-D.U
SEQ ID NO: 30
/56-FAM/CAG ACC ACC /ZEN/ UGC
UGG GAC CAC CCU /3IABkFQ/
DMD-PROBE-D.0
SEQ ID NO: 31
CAG ACC ACC TGC TGG GAC CAC CCT
DMD-PROBE-D.0.U
SEQ ID NO: 32
CAG ACC ACC UGC UGG GAC CAC CCU
DMD-FWD-E
SEQ ID NO: 63
CAG GAT GGG CTA CCT GCC CGT G
DMD-FWD-E.U
SEQ ID NO: 64
CAG GAU GGG CUA CCU GCC CGU G
DMD-FWD-F
SEQ ID NO: 65
CCT ACT ACA TCA ACC ACG AGA CC
DMD-FWD-F.U
SEQ ID NO: 66
CCU ACU ACA UCA ACC ACG AGA CC
DMD-REV-E
SEQ ID NO: 67
CAC TCT GAT CGA TGC ATC TGA GCT CTT
DMD-REV-E.U
SEQ ID NO: 68
CAC UCU GAU CGA UGC AUC UGA GCU CUU
DMD-REV-F
SEQ ID NO: 69
TAG GCC TCT CGA GCT CCT CAT CA
DMD-REV-F.U
SEQ ID NO: 70
UAG GCC UCU CGA GCU CCU CAU CA
DMD-REV-G
SEQ ID NO: 71
CTC TGA TAC AGC TCG GTC ATC TT
DMD-REV-G.U
SEQ ID NO: 72
CUC UGA UAC AGC UCG GUC AUC UU
DMD-PROBE-F
SEQ ID NO: 73
/56-FAM/ACC GTG CTG /ZEN/ GAA GGC
GAC AAC ATG GAG ACC /3IABkFQ/
DMD-PROBE-F.U
SEQ ID NO: 74
/56-FAM/ACC GUG CUG /ZEN/ GAA GGC
GAC AAC AUG GAG ACC /3IABkFQ/
DMD-PROBE-F.0
SEQ ID NO: 75
ACC GTG CTG GAA GGC GAC AAC ATG GAG ACC
DMD-PROBE-F.0.U
SEQ ID NO: 76
ACC GUG CUG GAA GGC GAC AAC AUG GAG ACC
DMD-PROBE-G
SEQ ID NO: 77
/56-FAM/CAG ACC ACC /ZEN/ TGC TGG GAC
CAC CCT /3IABkFQ/
DMD-PROBE-G.U
SEQ ID NO: 78
/56-FAM/CAG ACC ACC /ZEN/ UGC UGG
GAC CAC CCU /3IABkFQ/
DMD-PROBE-G.0
SEQ ID NO: 79
CAG ACC ACC TGC TGG GAC CAC CCT
DMD-PROBE-G.0.U
SEQ ID NO: 80
CAG ACC ACC UGC UGG GAC CAC CCU

Oligonucleotide Combinations for DMD AAV Specific Quantification

In some embodiments, primer combinations may be utilized for accurate and specific DMD AAV quantification.

In some embodiments, a combination of primers can comprise one or more forward primers. In some embodiments, a combination of primers can comprise one or more reverse primers. In some embodiments, one or more forward primers can comprise or consist of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 63, SEQ ID NO: 64, SEQ ID NO: 65, SEQ ID NO: 66, or a combination thereof. In some embodiments, one or more reverse primers can comprise or consist of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 67, SEQ ID NO: 68, SEQ ID NO: 69, SEQ ID NO: 70, SEQ ID NO: 71, SEQ ID NO: 72, or a combination thereof.

In some embodiments, a composition comprises a combination of primers and one or more probes. In some embodiments, one or more probes can comprise or consist of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, or a combination thereof.

In some embodiments, the primer combination comprises DMD-FWD-A (SEQ ID NO: 1) and DMD-REV-B (SEQ ID NO: 11). In some embodiments, the primer combination comprising DMD-FWD-A (SEQ ID NO: 1) and DMD-REV-B (SEQ ID NO: 11) is coupled with the probe sequence DMD-PROBE-A (SEQ ID NO: 17). In some embodiments, the primer combination comprising DMD-FWD-A (SEQ ID NO: 1) and DMD-REV-B (SEQ ID NO: 11) is coupled with the probe sequence DMD-PROBE-B (SEQ ID NO: 21).

In some embodiments, the primer combination comprises DMD-FWD-B (SEQ ID NO: 3), and DMD-REV-A (SEQ ID NO: 9). In some embodiments, the primer combination comprising DMD-FWD-B (SEQ ID NO: 3), and DMD-REV-A (SEQ ID NO: 9) is coupled with the probe sequence DMD-PROBE-A (SEQ ID NO: 17).

In some embodiments, the primer combination comprises DMD-FWD-E (SEQ ID NO: 63) and DMD-REV-E (SEQ ID NO: 67). In some embodiments, the primer combination comprising DMD-FWD-E (SEQ ID NO: 63) and DMD-REV-E (SEQ ID NO: 67) is coupled with the probe sequence DMD-PROBE-F (SEQ ID NO: 73).

In some embodiments, the primer combination comprises DMD-FWD-F (SEQ ID NO: 69) and DMD-REV-G (SEQ ID NO: 71). In some embodiments, the primer combination comprising DMD-FWD-F (SEQ ID NO: 69) and DMD-REV-G (SEQ ID NO: 71) is coupled with the probe sequence DMD-PROBE-G (SEQ ID NO: 77).

In some embodiments, the primer combination comprises DMD-FWD-B (SEQ ID NO:3) and DMD-REV-A (SEQ ID NO: 9). In some embodiments, the primer combination comprising DMD-DMD-FWD-B (SEQ ID NO:3) and DMD-REV-A (SEQ ID NO: 9) is coupled with the probe sequence DMD-PROBE-A (SEQ ID NO: 17).

In some embodiments, the primer combination comprises DMD-FWD-C (SEQ ID NO: 5) and DMD-REV-D (SEQ ID NO: 15). In some embodiments, the primer combination comprising DMD-FWD-C(SEQ ID NO: 5) and DMD-REV-D (SEQ ID NO: 15) is coupled with the probe sequence DMD-PROBE-D (SEQ ID NO: 29).

In some embodiments, the primer combination comprises DMD-FWD-D (SEQ ID NO: 7), and DMD-REV-C(SEQ ID NO: 13). In some embodiments, the primer combination comprising DMD-FWD-D (SEQ ID NO: 7), and DMD-REV-C(SEQ ID NO: 13) is coupled with the probe sequence DMD-PROBE-C(SEQ ID NO: 25).

Oligonucleotide Sequences for HEM-B AAV Specific Quantification

Each of the following Primer sequences is provided in 5′ to 3′ order; those skilled in the art will recognize than in certain embodiments a polynucleotide may be an RNA molecule, or a DNA molecule. In certain embodiments, primary screening comprised the evaluation of 72 primer combinations and 5 different probes, culminating in 47 different primer-probe combinations.

HemB-FWD-A
SEQ ID NO: 33
GTG GAG AGG AGC AGA GGT TGT C
HemB-FWD-A.U
SEQ ID NO: 34
GUG GAG AGG AGC AGA GGU UGU C
HemB-FWD-B
SEQ ID NO: 35
CAC TGC TTA AAT ACG GAC GAG GA
HemB-FWD-B.U
SEQ ID NO: 36
CAC UGC UUA AAU ACG GAC GAG GA
HemB-FWD-C
SEQ ID NO: 37
GAG GCA CCA CCA CTG ACC T
HemB-FWD-C.U
SEQ ID NO: 38
GAG GCA CCA CCA CUG ACC U
HemB-REV-A
SEQ ID NO: 39
CTG TTC CAC TGG TAG CAA GAT CC
HemB-REV-A.U
SEQ ID NO: 40
GUG UUC CAC UGG UAG CAA GAU CC
HemB-REV-B
SEQ ID NO: 41
ATG ATC ATG TTC ACC CTC TGC AT
HemB-REV-B.U
SEQ ID NO: 42
AUG AUC AUG UUC ACC CUC UGC AU
HemB-REV-C
SEQ ID NO: 43
CAT AAC CTT TGC TAG CAG ATT GTG
HemB-REV-C.U
SEQ ID NO: 44
CAU AAC CUU UGC UAG CAG AUU GUG ACG
HemB-REV-D
SEQ ID NO: 45
CTT TGC TAG GAG ATT GTG AAA GTG
HemB-REV-D.U
SEQ ID NO: 46
CXX XGC XAG CAG AXX GXG AAA GXG
HemB-PROBE-A
SEQ ID NO: 47
/56-FAM/CCC TCT CAC /ZEN/ACT ACC TAA ACC
ACG CCA /3IABkFQ/
HemB-PROBE-A.U
SEQ ID NO: 48
/56-FAM/CCC UCU CAC /ZEN/ACU ACC UAA ACC
GGC C/3IABkFQ/
HemB-PROBE-A.0
SEQ ID NO: 49
CCC TCT CAC ACT ACC TAA ACC ACG CCA
HemB-PROBE-A.0.U
SEQ ID NO: 50
CCC UCU CAC ACU ACC UAA ACC ACG CCA
HemB-PROBE-B
SEQ ID NO: 51
/56-FAM/TGC CTG AAG /ZEN/CTG AGG AGA CAG
HemB-PROBE-B.U GGC C/3IABkFQ/
SEQ ID NO: 52
/56-FAM/UGC CUG AAG /ZEN/CUG AGG AGA CAG
GGA C/3IABkFQ/
HemB-PROBE-B.0
SEQ ID NO: 53
TGC CTG AAG CTG AGG AGA CAG GGC C
HemB-PROBE-B.0.U
SEQ ID NO: 54
UGC CUG AAG CUG AGG AGA CAG GGC C
HemB-PROBE-C
SEQ ID NO: 55
/56-FAM/TCA GGC ACC /ZEN/ACC ACT GAC CTG
GGA C/3IABkFQ/
HemB-PROBE-C.U
SEQ ID NO: 56
/56-FAM/UCA GGC ACC /ZEN/ACC ACU GAC CUG
HemB-PROBE-C.0
SEQ ID NO: 57
TCA GGC ACC ACC ACT GAC CTG GGA C
HemB-PROBE-C.0.U
SEQ ID NO: 58
UCA GGC ACC ACC ACU GAC CUG GGA C
HemB-PROBE-D
SEQ ID NO: 59
/56-FAM/AGC AGA TTG /ZEN/TGA AAG
TGG TAT TCA CTG TCC /3IABkFQ/
HemB-PROBE-D.U
SEQ ID NO: 60
/56-FAM/AGC AGA UUG /ZEN/UGA AAG
UGG UAU UCA CUG UCC /3IABkFQ/
HemB-PROBE-D.0
SEQ ID NO: 61
AGC AGA TTG TGA AAG TGG TAT TCA CTG TCC
HemB-PROBE-D.0.U
SEQ ID NO: 62
AGC AGA UUG UGA AAG UGG UAU UCA CUG UCC
HemB-FWD-D
SEQ ID NO: 81
ACT GGA TTA AGG AGA AAA CCA AGC TG
HemB-FWD-D.U
SEQ ID NO: 82
ACU GGA UUA AGG AGA AAA CCA AGC UG
HemB-FWD-E
SEQ ID NO: 83
TGT GAA CTG GAT TAA GGA GAA AAC CA
HemB-FWD-E.U
SEQ ID NO: 84
UGU GAA CUG GAU UAA GGA GAA AAC CA
HemB-REV-E
SEQ ID NO: 85
TCT ATA TCT AAA AGG CAA GCA TAG TCA
HemB-REV-E.U
SEQ ID NO: 86
UCU AUA UCU AAA AGG CAA GCA UAC UCA
HemB-REV-F
SEQ ID NO: 87
GGC AAC TAG AAG GCA CAG TAG A
HemB-REV-E.U
SEQ ID NO: 88
GGC AAC UAG AAG GCA CAG UAG A
HemB-PROBE-E
SEQ ID NO: 89
/56-FAM/CTG CAG CCA /ZEN/ GGG
GGA TCA GCC /3IABkFQ/
HemB-PROBE-E.U
SEQ ID NO: 90
/56-EAM/CUG CAG CCA /ZEN/ GGG
GGA UCA GCC /3IABkFQ/
HemB-PROBE-E.0
SEQ ID NO: 91
CTG CAG CCA GGG GGA TCA GCC
HemB-PROBE-E.0.U
SEQ ID NO: 92
CUG CAG CCA GGG GGA UCA GCC

Oligonucleotide Combinations for HEM-B AAV Specific Quantification

In some embodiments, primer combinations may be utilized for accurate and specific Hem-B AAV quantification.

In some embodiments, a combination of primers can comprise one or more forward primers. In some embodiments, a combination of primers can comprise one or more reverse primers. In some embodiments, one or more forward primers can comprise or consist of SEQ ID NO: 33, SEQ ID NO: 34, SEQ ID NO: 35, SEQ ID NO: 36, SEQ ID NO: 37, SEQ ID NO: 38, SEQ ID NO: 81, SEQ ID NO: 82, SEQ ID NO: 83, SEQ ID NO: 84, or a combination thereof. In some embodiments, one or more reverse primers can comprise or consist of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 85, SEQ ID NO: 86, SEQ ID NO: 87, SEQ ID NO: 88, or a combination thereof.

In some embodiments, a composition comprises a combination of primers and one or more probes. In some embodiments, one or more probes can comprise or consist of SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 89, SEQ ID NO: 90, SEQ ID NO: 91, SEQ ID NO: 91, SEQ ID NO: 92, or a combination thereof.

In some embodiments, the primer combination comprises HemB-FWD-B (SEQ ID NO: 35) and HemB-REV-D (SEQ ID NO: 45). In some embodiments, the primer combination comprising HemB-FWD-B (SEQ ID NO: 35) and HemB-REV-D (SEQ ID NO: 45) is coupled with the probe sequence HemB-PROBE-C(SEQ ID NO: 55).

In some embodiments, the primer combination comprises HemB-FWD-A (SEQ ID NO: 33) and HemB-REV-A (SEQ ID NO: 39). In some embodiments, the primer combination comprising HemB-FWD-A (SEQ ID NO: 33) and HemB-REV-A (SEQ ID NO: 39) is coupled with the probe sequence HemB-PROBE-A (SEQ ID NO: 47).

In some embodiments, the primer combination comprises HemB-FWD-D (SEQ ID NO: 81) and HemB-REV-F (SEQ ID NO: 87). In some embodiments, the primer combination comprising HemB-FWD-D (SEQ ID NO: 81) and HemB-REV-F (SEQ ID NO: 87) is coupled with the probe sequence HemB-PROBE-E (SEQ ID NO:89).

In some embodiments, the primer combination comprises HemB-FWD-B (SEQ ID NO: 35) and HemB-REV-C(SEQ ID NO: 43). In some embodiments, the primer combination comprising HemB-FWD-B (SEQ ID NO: 35) and HemB-REV-C(SEQ ID NO: 43) is coupled with the probe sequence HemB-PROBE-B (SEQ ID NO: 51).

In some embodiments, the primer combination comprises HemB-FWD-C (SEQ ID NO: 37) and HemB-REV-B (SEQ ID NO: 41). In some embodiments, the primer combination comprising HemB-FWD-C(SEQ ID NO: 37) and HemB-REV-B (SEQ ID NO: 41) is coupled with the probe sequence HemB-PROBE-D (SEQ ID NO: 59).

In some embodiments, the primer combination comprises HemB-FWD-C (SEQ ID NO: 37) and HemB-REV-B (SEQ ID NO: 41). In some embodiments, the primer combination comprising HemB-FWD-C(SEQ ID NO: 37) and HemB-REV-B (SEQ ID NO: 41) is coupled with the probe sequence HemB-PROBE-D (SEQ ID NO: 59).

In some embodiments, the primer combination comprises HemB-FWD-E (SEQ ID NO: 83) and HemB-REV-F (SEQ ID NO: 85). In some embodiments, the primer combination comprising HemB-FWD-E (SEQ ID NO: 83) and HemB-REV-F (SEQ ID NO: 85) is coupled with the probe sequence HemB-PROBE-E (SEQ ID NO: 89).

### Oligonucleotide Preparation

Oligonucleotides of the present disclosure may be prepared by any of a variety of methods (see, e.g., Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, Innis (Ed.), Academic Press: New York, N.Y.; Tijssen “Hybridization with Nucleic Acid Probes—Laboratory Techniques in Biochemistry and Molecular Biology (Parts I and II)”, 1993, Elsevier Science; “PCR Strategies”, 1995, Innis (Ed.), Academic Press: New York, N.Y.; and “Short Protocols in Molecular Biology”, 2002, Ausubel (Ed.), 5^{th} Ed., John Wiley & Sons: Secaucus, N.J.).

In some embodiments, oligonucleotides may be prepared by chemical techniques well-known in the art, including, e.g., chemical synthesis and polymerization based on a template as described, e.g., in Narang et al., Meth. Enzymol. 68:90-98 (1979); Brown et al., Meth. Enzymol. 68: 109-151 (1979); Belousov et al., Nucleic Acids Res. 25:3440-3444 (1997); Guschin et al., Anal. Biochem. 250:203-211 (1997); Blommers et al., Biochemistry 33:7886-7896 (1994); Frenkel et al., Free Radic. Biol. Med. 19:373-380 (1995); and U.S. Pat. No. 4,458,066.

In some embodiments, oligonucleotides may be prepared using an automated, solid-phase procedure based on the phosphoramidite approach. In such methods, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of oligonucleotides, where necessary or desirable, may be carried out by any of a variety of methods well-known in the art. For example, purification of oligonucleotides is typically performed either by native acrylamide gel electrophoresis, by anion-exchange HPLC, e.g., see Pearson and Regnier, J. Chrom. 255:137-149 (1983) or by reverse phase HPLC, e.g., see McFarland and Borer, Nucleic Acids Res. 7:1067-1080 (1979).

The sequence of oligonucleotides can be verified using any suitable sequencing method including, but not limited to, chemical degradation, e.g., see Maxam and Gilbert, Methods of Enzymology, 65:499-560 (1980), matrix-assisted laser desorption ionization time-of-flight (MALDI-TOF) mass spectrometry, e.g., see Pieles et al., Nucleic Acids Res. 21:3191-3196 (1993), mass spectrometry following a combination of alkaline phosphatase and exonuclease digestions, e.g., see Wu and Aboleneen, Anal. Biochem. 290:347-352 (2001).

The present disclosure encompasses modified versions of these oligonucleotides that perform as equivalents of these oligonucleotides in accordance with the methods of the present disclosure. These modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.), or charged linkages (e.g., phosphorothioates, phosphorodithioates, etc.). Modified oligonucleotide may also be derivatized by formation of a methyl or ethyl phosphotriester or an alkyl phosphoramidate linkage. Furthermore, the oligonucleotides of the present disclosure may also be modified with a label.

### Labeling of Oligonucleotides

In some embodiments, the primers are labeled with a detectable agent or moiety before being used in amplification/detection assays. The role of a detectable agent is to allow visualization and detection of amplified target sequences. Preferably, the detectable agent is selected such that it generates a signal which can be measured and whose intensity is related (e.g., proportional) to the amount of amplification products in the sample being analyzed.

The association between the oligonucleotide and the detectable agent can be covalent or non-covalent. Labeled detection primers can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules, e.g., see Mansfield et al., Mol. Cell Probes 9:145-156 (1995).

Various methods for labeling nucleic acid molecules are known in the art. For a review of labeling protocols, label detection techniques, and recent developments in the field, see, for example, Kricka, Ann. Clin. Biochem. 39:114-129 (2002); van Gijlswijk et al., Expert Rev. Mol. Diagn. 1:81-91 (2001); and Joos et al., J. Biotechnol. 35:135-153 (1994). Standard nucleic acid labeling methods include: incorporation of radioactive agents, direct attachments of fluorescent dyes (Smith et al., Nucl. Acids Res. 13:2399-2412 (1985)) or of enzymes (Connoly and Rider, Nucl. Acids. Res. 13:4485-4502 (1985)); chemical modifications of nucleic acid molecules making them detectable immunochemically or by other affinity reactions, e.g., see Broker et al., Nucl. Acids Res. 5:363-384 (1978); Bayer et al., Methods of Biochem. Analysis 26:1-45 (1980); Langer et al., Proc. Natl. Acad. Sci. USA 78:6633-6637 (1981); Richardson et al., Nucl. Acids Res. 11:6167-6184 (1983); Brigati et al., Virol. 126:32-50 (1983); Tchen et al., Proc. Natl. Acad. Sci. USA 81:3466-3470 (1984); Landegent et al., Exp. Cell Res. 15:61-72 (1984); and Hopman et al., Exp. Cell Res. 169:357-368 (1987); and enzyme-mediated labeling methods, such as random priming, nick translation, PCR and tailing with terminal transferase. For a review on enzymatic labeling, see, e.g., Temsamani and Agrawal, Mol. Biotechnol. 5:223-232 (1996). More recently developed nucleic acid labeling systems include, but are not limited to: ULS (Universal Linkage System), which is based on the reaction of monoreactive cisplatin derivatives with the N7 position of guanine moieties in DNA (Heetebrij et al., Cytogenet. Cell. Genet. 87:47-52 (1999)), psoralen-biotin, which intercalates into nucleic acids and upon UV irradiation becomes covalently bonded to the nucleotide bases (Levenson et al., Methods Enzymol. 184:577-583 (1990); and Pfannschmidt et al., Nucleic Acids Res. 24:1702-1709 (1996)), photoreactive azido derivatives (Neves et al., Bioconjugate Chem. 11:51-55 (2000)), and DNA alkylating agents (Sebestyen et al., Nat. Biotechnol. 16: 568-576 (1998)).

It will be appreciated that any of a wide variety of detectable agents can be used in the practice of the present disclosure. Suitable detectable agents include, but are not limited to, various ligands, radionuclides (such as, for example, ³²P, ³⁵S, ³H, ¹⁴C, ¹²⁵I, ¹³¹I and the like); fluorescent dyes (such as, for example, FAM, Yakima Yellow®, SUN™, HEX, Cy® 3, Texas Red®-X, and/or Cy® 5); fluorescent dye quenchers (such as, for example, ZEN, Iowa Black™ FQ, Iowa Black RQ, and/or TAO), chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, e.g., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

A “tail” of normal or modified nucleotides can also be added to tag an oligonucleotide for detectability purposes. In some embodiments, an M13 tag sequence may be added.

Extraction and Preparation of Viral Oligonucleotides from Biological Samples

In some embodiments, materials and methods of the present disclosure may include oligonucleotides extracted and prepared from biological samples. In some embodiments, suitable biological samples for the extraction of oligonucleotides include but are not limited to: urine, semen, plasma, stool, whole blood, and/or saliva. The extraction and preparation of oligonucleotides from biological samples occurs through methods generally known in the art (e.g., VERSANT® kPCR Sample Preparation 1.0 and/or 1.2).

### Amplification Methods and Reactions

In some embodiments, the present disclosure provides methods that use the aforementioned oligonucleotides as amplification primers to amplify regions of specific AAV constructs, in particular regions that are not found in natural AAV, and are specific to certain therapeutic AAVs. As discussed in more detail below, in some embodiments the primers are used in quantitative PCR methods for the amplification and detection of specific AAV constructs.

The use of oligonucleotide sequences of the present disclosure as primers to amplify AAV target sequences in test samples is not limited to any particular nucleic acid amplification technique or any particular modification thereof. In fact, the inventive oligonucleotide sequences can be employed in any of a variety of nucleic acid amplification methods well-known in the art (see, for example, Kimmel and Berger, Methods Enzymol. 152: 307-316 (1987); Sambrook et al., “Molecular Cloning: A Laboratory Manual”, 1989, 2^{nd} Ed., Cold Spring Harbour Laboratory Press: New York, N.Y.; “Short Protocols in Molecular Biology”, Ausubel (Ed.), 2002, 5^{th} Ed., John Wiley & Sons: Secaucus, N.J.).

Such nucleic acid amplification methods include, but are not limited to, the Polymerase Chain Reaction (or PCR, described, for example, in “PCR Protocols: A Guide to Methods and Applications”, Innis (Ed.), 1990, Academic Press: New York; “PCR Strategies”, Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature 324:163 (1986); and U.S. Pat. Nos. 4,683,195, 4,683,202 and 4,889,818, each of which is incorporated herein by reference in its entirety); and reverse transcriptase polymerase chain reaction (or RT-PCR, described in, for example, U.S. Pat. Nos. 5,322,770 and 5,310,652).

The PCR (or polymerase chain reaction) technique is well-known in the art and has been disclosed, for example, in Mullis and Faloona, Methods Enzymol., 155:350-355 (1987). In its simplest form, PCR is an in vitro method for the enzymatic synthesis of specific DNA sequences, using two primers that hybridize to opposite strands and flank the region of interest in the target DNA. A plurality of reaction cycles, each cycle comprising: a denaturation step, an annealing step, and a polymerization step, results in the exponential accumulation of a specific DNA fragment, see for example, “PCR Protocols: A Guide to Methods and Applications”, Innis (Ed.), 1990, Academic Press: New York; “PCR Strategies”, Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature 324:163-166 (1986). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq) which are available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually about 90° C. to about 94° C. for a period of time ranging from about 3 to about 120 seconds, usually from about 5 to about 30 seconds. In some embodiments, the first cycle is preceded by an elongated denaturation step ranging from about 1 to 10 minutes, usually from about 2 to 5 minutes.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 45° C. to about 75° C., usually from about 50° C. to about 70° C., and more usually from about 53° C. to about 55° C. Annealing conditions are generally maintained for a period of time ranging from about 15 seconds to about 30 minutes, usually from about 30 seconds to about 1 minute.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes. In some embodiments, the final extension step is followed by an elongated extension step ranging from ranging from about 1 to 10 minutes, usually from about 2 to 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610. Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

In some embodiments, one or both of the PCR reactions are “kinetic PCR” (kPCR) or “kinetic RT-PCR” (kRT-PCR), which are also referred to as “real-time PCR” and “real-time RT-PCR,” respectively. These methods involve detecting PCR products via a probe that provides a signal (typically a fluorescent signal) that is related to the amount of amplified product in the sample. Examples of commonly used probes used in kPCR and kRT-PCR include the following probes: TAQMAN® probes, Molecular Beacons probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quench molecules prevents the detection of fluorescent signal from the probe. During PCR, when the polymerase replicates a template on which a probe is bound, the 5′-nuclease activity of the polymerase cleaves the probe thus, increasing fluorescence with each replication cycle. SYBR® Green probes binds double-stranded DNA and upon excitation emit light; thus as PCR product accumulates, fluorescence increases.

In some embodiments, the PCR reaction is used in a “single-plex” PCR assay. “Single-plex” refers to a single assay that is not carried out simultaneously with any other assays. Single-plex assays include individual assays that are carried out sequentially.

In some embodiments, the PCR reaction is used in a “multiplex” PCR assay. The term “multiplex” refers to multiple assays that are carried out simultaneously, in which detection and analysis steps are generally performed in parallel. Within the context of the present disclosure, a multiplex assay will include the use of the primers, alone or in combination with additional primers to identify, for example, an internal control, or an HCV virus variant along with one or more additional HCV variants or other viruses.

In some embodiments, a first amplification step amplifies a region of a target gene. In some embodiments the amplification product is less than about 1500, 1400, 1300, 1200, 1100, 1000, 900, 800, 700, 600, 500, 400, 300, 250, 225, 200, 175, 150, 125, 100, or 75 nucleotides long.

### Detection of Amplification Products

Amplification products generated using the oligonucleotides and methods of the present disclosure may be detected using a variety of methods known in the art.

In some embodiments, amplification products may simply be detected using agarose gel electrophoresis and visualization by ethidium bromide staining and exposure to ultraviolet (UV) light.

In some embodiments, the presence of a specific genotype can be shown by restriction enzyme analysis. For example, a specific nucleotide polymorphism can result in a nucleotide sequence comprising a restriction site which is absent from the nucleotide sequence of another allelic variant. Additionally or alternately, a specific nucleotide polymorphism can result in the elimination of a nucleotide sequence comprising a restriction site which is present in the nucleotide sequence of another allelic variant.

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally and then hybridized to target DNA under conditions which permit hybridization only if a perfect match is found, e.g., see Saiki et al., Nature 324:163 (1986); Saiki et al., Proc. Natl Acad. Sci USA 86:6230 (1989); and Wallace et al., Nucl. Acids Res. 6:3543 (1979). Such specific oligonucleotide hybridization techniques may be used for the simultaneous detection of several nucleotide changes in different polymorphic regions of DNA. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. Alternatively unlabeled sample nucleic acid may be immobilized and contacted with labeled oligonucleotides that hybridize selectively with specific allelic variants.

Real-time pyrophosphate DNA sequencing is yet another approach to detection of polymorphisms and polymorphic variants, e.g., see Alderborn et al., Genome Research, 10(8):1249-1258 (2000). Additional methods include, for example, PCR amplification in combination with denaturing high performance liquid chromatography (dHPLC), e.g., see Underhill et al., Genome Research, 7(10):996-1005 (1997).

In some embodiments, any of a variety of sequencing reactions known in the art can be used to directly sequence at least a portion of amplified DNA and detect allelic variants. The sequence can be compared with the sequences of known allelic variants to determine which one(s) are present in the sample. Exemplary sequencing reactions include those based on techniques developed by Maxam and Gilbert, Proc. Natl. Acad. Sci USA, 74:560 (1977) or Sanger, Proc. Nat. Acad. Sci 74:5463 (1977). It is also contemplated that any of a variety of automated sequencing procedures may be utilized when performing the subject assays, e.g., see Venter et al., Science, 291:1304-1351 (2001); Lander et al., Nature, 409:860-921 (2001), including sequencing by mass spectrometry, e.g., see U.S. Pat. No. 5,547,835 and PCT Patent Publication No. WO 94/16101 and WO 94/21822; U.S. Pat. No. 5,605,798 and PCT Patent Application No. PCT/US96/03651; Cohen et al., Adv. Chromatogr. 36:127-162 (1996); and Griffin et al., Appl. Biochem. Biotechnol. 38:147-159 (1993). It will be evident to one skilled in the art that, for some embodiments, the occurrence of only one, two or three of the nucleic acid bases need be determined in the sequencing reaction. Yet other sequencing methods are disclosed, e.g., in U.S. Pat. Nos. 5,580,732; 5,571,676; 4,863,849; 5,302,509; PCT Patent Application Nos. WO 91/06678 and WO 93/21340; Canard et al., Gene 148:1-6 (1994); Metzker et al., Nucleic Acids Research 22:4259-4267 (1994) and U.S. Pat. Nos. 5,740,341 and 6,306,597.

In some embodiments, detection of an amplicon is performed using Real-time PCR. Real-time PCR has been developed to quantify amplified products during PCR reactions. Real-time PCR is based on the principles that emission of fluorescence from dyes directly or indirectly associated with the formation of newly-synthesized amplicons or the annealing of primers with DNA templates can be detected and is proportional to the amount of amplicons in each PCR cycle. Real-time PCR is carried out in a closed-tube format and is quantitative. Several methods are currently available for performing real-time PCR, such as utilizing TaqMan probes (U.S. Pat. Nos. 5,210,015 and 5,487,972, and Lee et al., Nucleic Acids Res. 21:3761-6, 1993), molecular beacons (U.S. Pat. Nos. 5,925,517 and 6,103,476, and Tyagi and Kramer, Nat. Biotechnol. 14:303-8, 1996), self-probing amplicons (scorpions) (U.S. Pat. No. 6,326,145, and Whitcombe et al., Nat. Biotechnol. 17:804-7, 1999), Amplisensor (Chen et al., Appl. Environ. Microbiol. 64:4210-6, 1998), Amplifluor (U.S. Pat. No. 6,117,635, and Nazarenko et al., Nucleic Acids Res. 25:2516-21, 1997, displacement hybridization probes (Li et al., Nucleic Acids Res. 30:E5, 2002), DzyNA-PCR (Todd et al., Clin. Chem. 46:625-30, 2000), fluorescent restriction enzyme detection (Cairns et al. Biochem. Biophys. Res. Commun. 318:684-90, 2004) and adjacent hybridization probes (U.S. Pat. No. 6,174,670 and Wittwer et al., Biotechniques 22:130-1, 134-8, 1997). Most of these probes consist of a pair of dyes (a reporter dye and an acceptor dye) that are involved in fluorescence resonance energy transfer (FRET), whereby the acceptor dye quenches the emission of the reporter dye. In general, the fluorescence-labeled probes increase the specificity of amplicon quantification.

In some embodiments, detection of an amplicon is performed using Real-time PCR coupled with a TaqMan assay. U.S. Pat. Nos. 5,210,015 and 5,487,972 describe the 5′ nuclease assay, also termed TaqMan assay. The TaqMan assay exploits the 5′ nuclease activity of Taq DNA Polymerase to cleave a TaqMan probe during PCR. The TaqMan probe contains a reporter dye at the 5′ end of the probe and a quencher dye at the 3′ end of the probe. During the reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the close proximity of the reporter dye to the quencher dye results in suppression of the reporter fluorescence primarily by Förster-type energy transfer (Förster, 1948; Lakowicz, 1983). During PCR, if the target of interest is present, the probe specifically anneals between the forward and reverse primer sites. The 5′ to 3′ nucleolytic activity of the Taq DNA polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of the product.

### Compositions and Kits

In some embodiments, the present disclosure provides kits comprising materials useful for the amplification and detection or sequencing of specific AAV constructs according to methods described herein. The inventive kits may be used by diagnostic laboratories, experimental laboratories, or practitioners.

Materials and reagents useful for the detection or sequencing of specific AAV constructs according to the present disclosure may be assembled together in a kit. In some embodiments, an inventive kit comprises at least one inventive primer set, and optionally, reverse transcription and/or amplification reaction reagents. In some embodiments, a kit comprises reagents which render the procedure specific. Thus, a kit intended to be used for the detection of a particular specific AAV construct variant preferably comprises primer sets described herein that can be used to amplify a particular specific AAV construct target sequence of interest. A kit intended to be used for the multiplex detection of a plurality of specific AAV construct target sequences and/or other viruses preferably comprises a plurality of primer sets (optionally in separate containers) described herein that can be used to amplify specific AAV construct target sequences described herein.

Suitable reverse transcription/amplification reaction reagents that can be included in an inventive kit include, for example, one or more of: buffers; enzymes having reverse transcriptase and/or polymerase activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and deoxythymidine triphosphate, biotinylated dNTPs, suitable for carrying out the amplification reactions.

Depending on the procedure, the kit may further comprise one or more of: wash buffers and/or reagents, hybridization buffers and/or reagents, labeling buffers and/or reagents, and detection means. The buffers and/or reagents included in a kit are preferably optimized for the particular amplification/detection technique for which the kit is intended. Protocols for using these buffers and reagents for performing different steps of the procedure may also be included in the kit.

Furthermore, the kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

Kits may also contain reagents for the isolation of nucleic acids from biological specimen prior to amplification and/or for the purification or separation of AAV particles before nucleic acid extraction.

The reagents may be supplied in a solid (e.g., lyophilized) or liquid form. The kits of the present disclosure optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents and primer sets or primer/probe sets according to the present disclosure. Instructions for using the kit according to one or more methods of the present disclosure may comprise instructions for processing the biological sample, extracting nucleic acid molecules, and/or performing the test; instructions for interpreting the results as well as a notice in the form prescribed by a governmental agency (e.g., FDA) regulating the manufacture, use or sale of pharmaceuticals or biological products.

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

For example, other assays, including those described in the Example section herein as well as those that are known in the art, can also be used in accordance with the present disclosure.

### EXAMPLES

### Reagents, Equipment, Methods, and Consumables Utilized For Immediate Examples—Reagents

| | | | Storage |
|---|---|---|---|
| Reagent | Source | Catalog # | Condition |
| MNase (2000 U/μL) | New | M0247S | −10° C. to |
| | England | | −30° C. |
| | Biosciences | | |
| | (NEB) | | |
| 0.5M EDTA, pH 8.0 | ThermoFisher | 15575020 | RT |
| Nuclease-free water | Ambion | AM9937 | RT |
| 100X BSA | NEB | B9001S | −10° C. to |
| | | | −30° C. |
| 10X MNase buffer | NEB | B0247S | −10° C. to |
| | | | −30° C. |
| Pre-treatment buffer | In-house | BKY-BPR-0003 | RT |
| Molecular Grade, | | MLS | RT |
| Nuclease Free Water | | | |
| PerfeCTa Toughmix, | QuantaBio | 95140-050K | −10° C. to |
| UNG, low ROX | | | −30° C. |
| 20X Hem-B Oligo Mix | Siemens | BKY-BPR-0002 | −10° C. to |
| | | | −30° C. |
| VERSANT ® kPCR | Siemens | Box 1 10286026 | RT |
| Sample Preparation | Healthcare | Box 2 10286027 | 2 to 8° C. |
| 1.0 Reagents | Diagnostics Inc | | |
| Siemens Sample | In-house | NA | 2° C. to 8° C. |
| Diluent 1 (SSD1) | | | |
| Siemens Storage | In-house | NA | 2° C. to 8° C. |
| Buffer 2 (SSB2) | | | |
| Siemens Pretreatment | In-house | NA | RT |
| Buffer (SPB) | | | |

### Equipment and Consumables

| Description | Vendor | Part # |
|---|---|---|
| Single-channel adjustable pipettes | MLS | N/A |
| (L10, L20, L200, and L1000) | | |
| Freezer (−10° C. to −30° C.) | MLS | NA |
| Bleach, unscented (0.5% sodium | MLS | NA |
| hypochlorite) | | |
| Incubator | MLS | NA |
| LTS 20 μL Filter Tips | Rainin | 17014961 |
| LTS 200 μL Filter Tips | Rainin | 17014963 |
| LTS 1000 μL Filter Tips | Rainin | 17014967 |
| 3.5 mL Sarstedt Tubes with | VWR | 101093-666 |
| enclosed neutral cap | | |
| Microcentrifuge tubes | MLS | NA |
| (0.5, 1.5, or 2.0 mL) | | |
| Amber Microcentrifuge tubes (2.0 mL) | MLS | NA |
| MicroAmp ® Optical 96-well | Applied | 4346907 |
| Fast reaction plate, 0.1 mL, | | |
| MicroAmp ® Optical Adhesive Film, | Applied | 4311971 |
| 100 each | | |
| Dedicated single-channel adjustable | MLS | NA |
| pipettes (P2, P10, P20, P200, and P1000) | | |
| Multi-channel pipette (P10, P20) | MLS | NA |
| Personal Protective Equipment (PPE) | MLS | NA |
| Dead-air hood with UV light | MLS | NA |
| Microcentrifuge | MLS | NA |
| Vortex Mixer | MLS | NA |
| Allegra 25R Centrifuge, 60 Hz, 208 V | Beckman | |
| | Coulter | |
| QuantStudio ™ 7 Flex Real-Time or DX | Thermo | |
| PCR System with 96-Well Fast Block | Fisher | |
| S5700 Swinging Bucket Rotor for | Beckman | 369434, 368954 |
| 96-well plate with microplate carrier | Coulter | |
| Refrigerator (2° C. to 8° C.) | MLS | NA |
| Sub-Freezer (−60° C. to −90° C.) | MLS | NA |
| VERSANT ® kPCR Molecular | SHDI | NA |
| System Sample Preparation | | |
| Module (SP Module) | | |
| Biosafety Cabinet | MLS | NA |
| SP1.0 Reagent Trough | SHDI | SMN 10489008 |
| (200 mL & 50 mL) | | |
| 1000-μL pipette tips | SHDI | SMN 10282929 |
| 300-μL pipette tips | SHDI | SMN 10282930 |
| 96-well, 2-mL nuclease free, | SHDI | SMN 10283255 |
| sterile deep well plates | | |
| Barcoded 96-well semi-skirted | SHDI | SMN 10282998 |
| polypropylene plates for | | |
| kPCR PCR Adapter Plate | SHDI | 96067-01 |
| Blue Absorbent Pads (bench protectors) | MLS | NA |
| 70% ethanol | MLS | NA |
| Bleach, unscented | MLS | NA |
| Microcide SQ | SHDI | SMN 10361387 |
| Deionized water | In-house | |
| Hamilton Tip Disposal Bags (200 ea.) | SHDI | SMN 10282938 |

### EXAMPLE 1: General Methodologies

The present example provides an overview of a number of generalizable assays and/or treatments utilized during sample preparation and quantification.

MNase Treatment—MNase Treatment procedure was performed prior to sample extraction and in line with manufacturer recommendations.

Quantification by qPCR for the AAV Shedding Assays—Extracted DNA was tested in a qPCR reaction using 54, of DNA extract. The concentration of AAV viral DNA in samples was determined by plotting their Ct values against Ct values obtained from extracted AAV viral calibrators in buffer at known concentrations. QuantStudio™ 7 Flex or Dx Real-Time PCR Systems were utilized for qPCR analysis. The concentration of AAV viral DNA in plasma, urine, semen and saliva samples were reported as viral genomes (vg)/mL and in PBMC samples reported as viral genomes (vg)/1 μg genomic DNA.

Sample Preparation for AAV Shedding Assay—Samples were prepared utilizing viral DNA extraction and purification processes according to the VERSANT® kPCR Molecular System—Stand Alone Sample Preparation module (SASP). Viral DNA samples were separated and purified for use in amplification procedures according to manufacturer instructions.

### EXAMPLE 2: Identifying and Optimizing Primers for Duchenne Muscular Dystrophy AAV Gene Therapy Shedding Quantification

The present example demonstrates the utility, accuracy, sensitivity, and precision of assays performed utilizing oligonucleotide sequences described herein. This shedding assay detects and quantifies an exemplary viral DNA (e.g., exemplary DMD AAV construct) extracted from urine, whole blood, stool and saliva. Exemplary DMD AAV construct is an adeno-associated virus encoding mini-dystrophin.

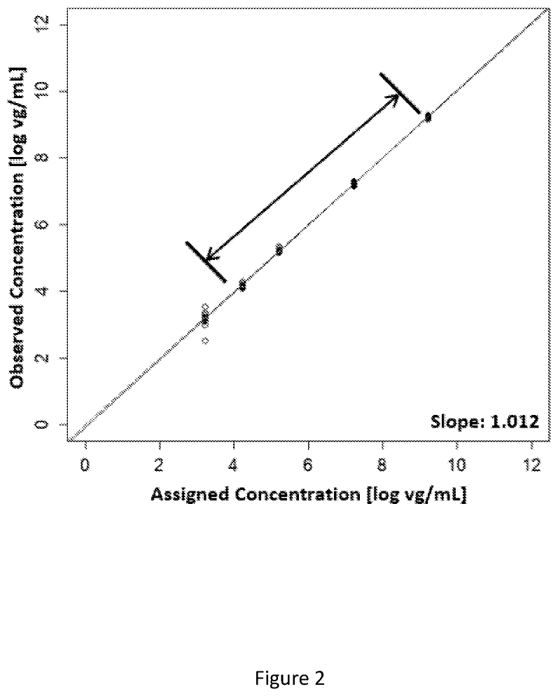

Extracted DNA was tested in a qPCR reaction using 5 μL of DNA extract. A linear regression curve was generated by plotting Ct values of extracted exemplary DMD AAV construct viral calibrators in buffer against their known concentrations (log transformed, see FIG. 2). The concentration of exemplary DMD AAV construct viral DNA in samples was determined by fitting their Ct values in this model. The concentration of exemplary DMD AAV construct viral DNA in urine, whole blood, and saliva samples were reported as viral genomes (vg)/mL and stool samples were reported as vg/mg.

Present FDA guidance requires qPCR to be able to detect<50 copies of vector/1 μg genomic DNA with 95% confidence. The qPCR quantitation limit was determined using a panel of linearized plasmid containing the exemplary DMD AAV construct viral genome spiked into human genomic DNA. N=444 was chosen for 25 copies of vector/1 μg genomic DNA to ensure that a 95% detection rate with enough confidence could be achieved (summarized below).

Primary Screening of Exemplary Primers and/or Probes

Primers were designed targeting select regions of an exemplary DMD AAV construct (see FIG. 1). Prior to complete oligonucleotide characterization, at least one or more forward primer, at least one or more reverse primer, and at least one or more probes were screened for characteristics such as multiplexing capacity, maximum fluorescence, slope, minimum CT, Tm, Oligonucleotide cross reactivity, and/or specificity for DMD AAV constructs in the presence of background genomic DNA.

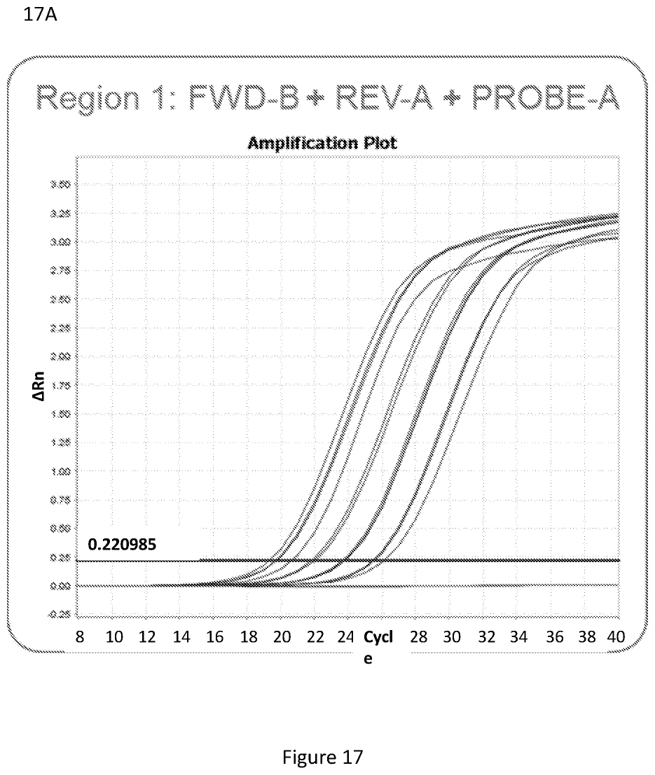

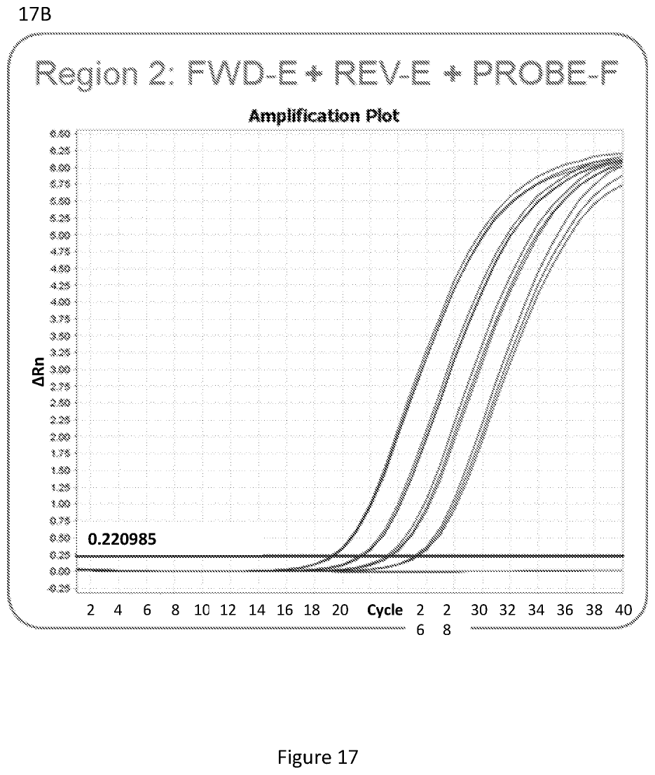

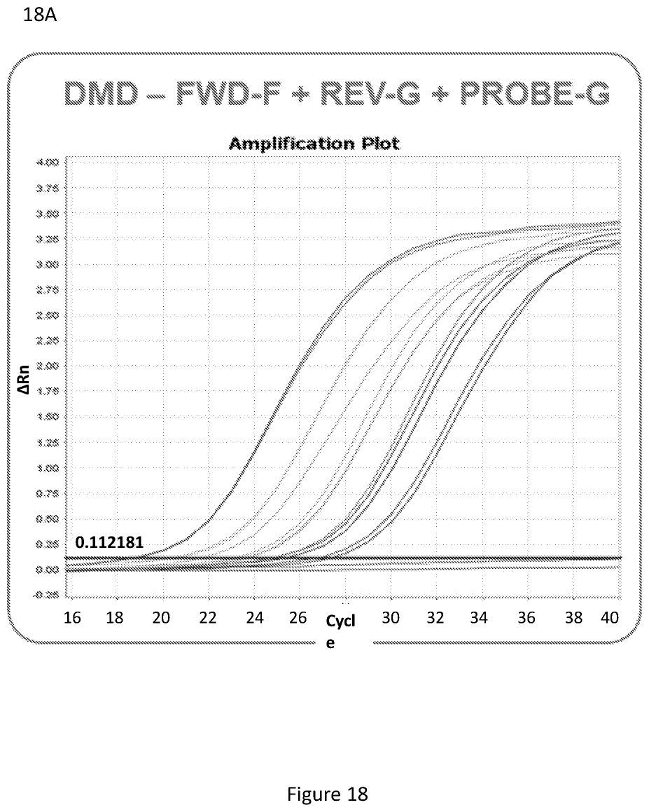

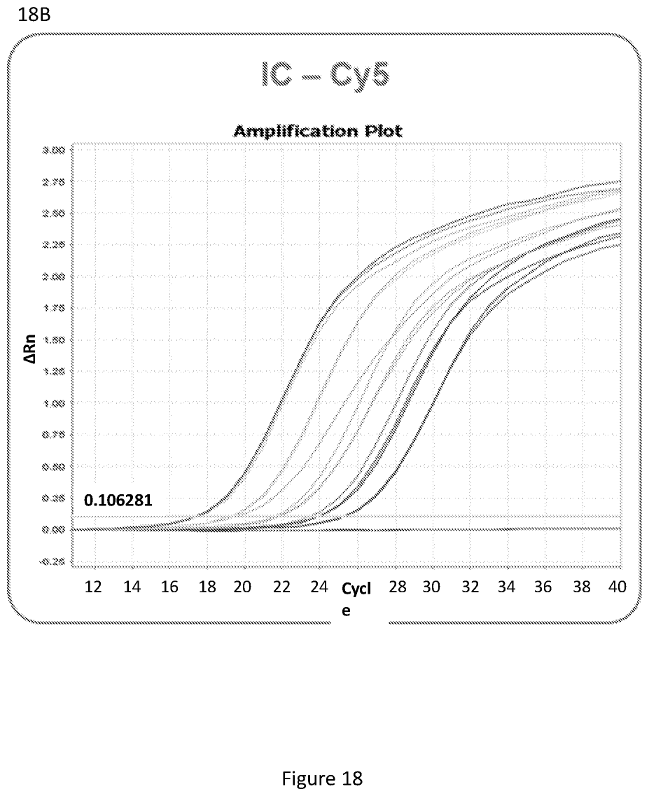

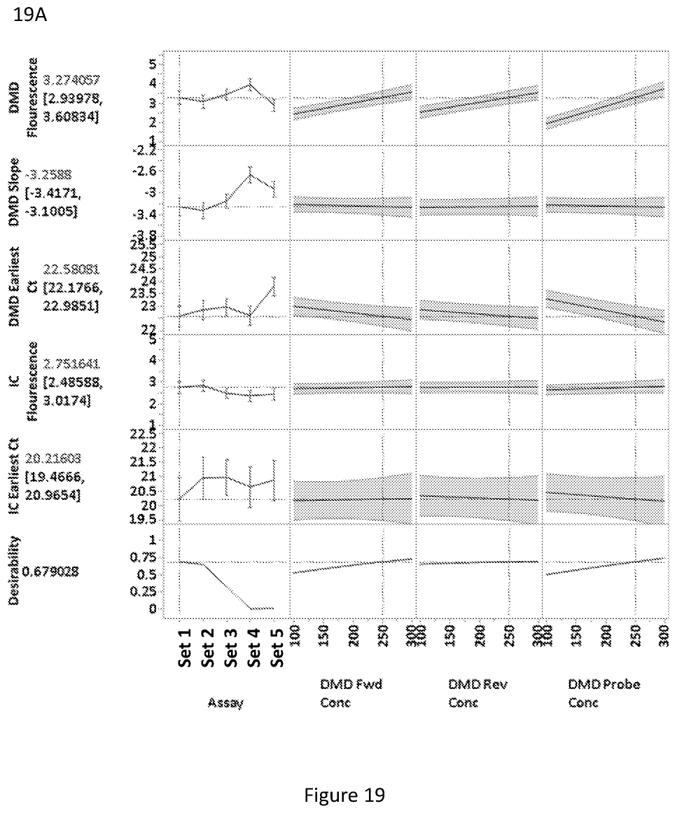

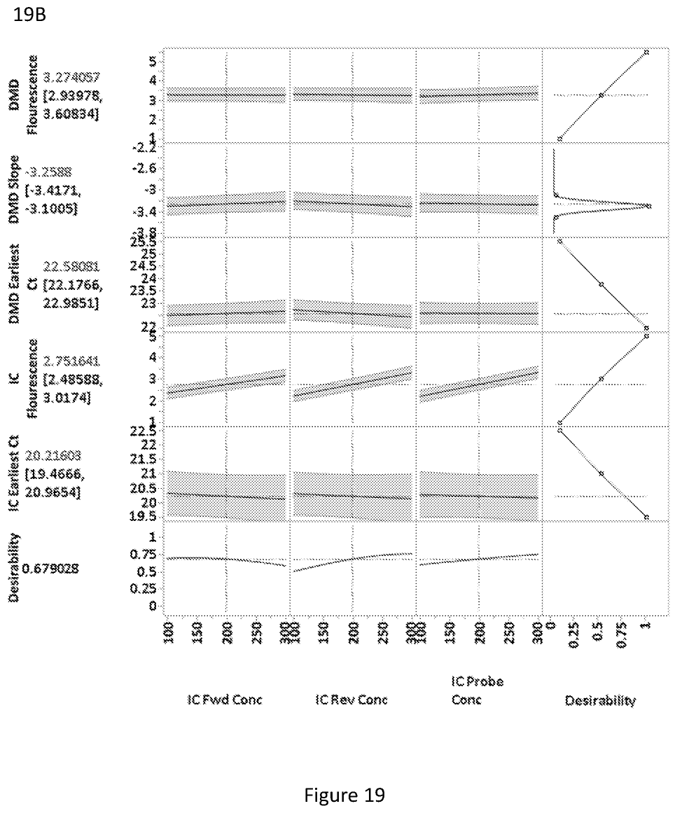

Certain primer and probe combinations were determined to work well under tested assay conditions. As shown in the figures and further herein, certain primer and probe combinations provided a desirable profile. However, alternative primer and/or probe combinations can be screened as shown to determine desirable profiles for other conditions, samples, etc., and the present disclosure recognizes that multiple primer and/or probe combinations may be suitable for accurate and reliable detection and/or quantification. The data also confirms that select primer and probe combinations work well when multiplexed, for example, with internal control primer and probe combinations. In some embodiments, those of skill in the art will understand that certain characteristics may be more or less desirable and that selection of a set of primers and probes for a particular application may be dependent on the weighing of certain characteristics such as multiplexing capacity, maximum fluorescence, slope, minimum CT, Tm, oligonucleotide cross reactivity, specificity for DMD AAV constructs in the presence of background genomic DNA, or any combination of all or any of these factors. Nonetheless, the data provided herein established that the disclosed primers and/or probes worked well for the detection of AAV constructs in biological samples. At least one or more forward primers, at least one or more reverse primers, and at least one or more probes were found suitable for one or more primary screening criteria (see e.g., Table 1, Table 2, and FIGS. 17-19).

TABLE 1
Primary Screening of Certain Oligonucleotide
Combinations targeting alternative
Exemplary DMD AAV Construct regions.

| | Exem- | | | | | |
|---|---|---|---|---|---|---|
| | plary | | | | | |
| | DMD | | | | | |
| | AAV | | | | | |
| | Con- | | | | Amplicon | |
| | struct | | | | Size | |
| Assay | Region | Fwd-P | Rev-P | Detection | [bp] | |
| 1 | Region | DMD- | DMD- | DMD- | 98 | |
| | 1 | FWD-A | REV-B | PROBE-A | | |
| | | (SEQ ID | (SEQ ID | (SEQ ID | | |
| | | NO: 1) | NO: 11) | NO: 17) | | |
| 2 | | DMD- | DMD- | DMD- | 95 | |
| | | FWD-B | REV-A | PROBE-A | | |
| | | (SEQ ID | (SEQ ID | (SEQ ID | | |
| | | NO: 3) | NO: 9) | NO: 17) | | |
| 3 | Region | DMD- | DMD- | DMD- | 108 | |
| | 2 | FWD-E | REV-E | PROBE-F | | |
| | | (SEQ ID | (SEQ ID | (SEQ ID | | |
| | | NO: 63) | NO: 67) | NO: 73) | | |
| 4 | | DMD- | DMD- | DMD- | 78 | |
| | | FWD-E | REV-F | PROBE-F | | |
| | | (SEQ ID | (SEQ ID | (SEQ ID | | |
| | | NO: 63) | NO: 69) | NO: 73) | | |
| 5 | Region | DMD- | DMD- | DMD- | 70 | |
| | 3 | FWD-F | REV-G | PROBE-G | | |
| | | (SEQ ID | (SEQ ID | (SEQ ID | | |
| | | NO: 65) | NO: 71) | NO: 77) | | |
| | | | | | | Ampl. |
| | | | | | | effi- |
| | 62500 | 15625 | 3906.25 | 976.56 | | cien- |
| Assay | copies | copies | copies | copies | slope | cy |
| 1 | 19.50 | 21.48 | 23.48 | 25.77 | −3.45 | 94.8 |
| 2 | 19.62 | 21.55 | 23.77 | 25.73 | −3.41 | 96.3 |
| 3 | 19.31 | 21.38 | 23.35 | 25.32 | −3.32 | 99.9 |
| 4 | 19.36 | 21.38 | 23.41 | 25.57 | −3.43 | 95.7 |
| 5 | 16.97 | 19.27 | 21.10 | 23.51 | −3.56 | 90.90 |

TABLE 2
Primary Screening of Certain Multiplex
Assays for Exemplary DMD AAV Constructs

| | | | | | Amplicon |
|---|---|---|---|---|---|
| As- | DNA | | | Detec- | Size |
| say | Source | Fwd-P | Rev-P | tion | [bp] |
| 1 | Exem- | DMD- | DMD- | DMD- | 70 |
| | plary | FWD-F | REV-G | PROBE-G | |
| | DMD | (SEQ ID | (SEQ ID | (SEQ ID | |
| | AAV | NO: 65) | NO: 71) | NO: 77) | |
| | Con- | | | | |
| | struct | | | | |
| 2 | IC | IC-F | IC-R | IC-P | — |

| As- | 62500 | 15625 | 3906.25 | 976.56 | 488.28 | | Ampl. |
|---|---|---|---|---|---|---|---|
| say | copies | copies | copies | copies | copies | slope | efficiency |
| 1 | 18.72 | 20.85 | 23.47 | 25.01 | 27.02 | −3.57 | 90.66 |
| 2 | 17.56 | 19.48 | 21.84 | 23.88 | 25.37 | −3.54 | 91.72 |

### Data Calculations and Reporting

The observed data were fitted with a nonlinear regression model as follows:

Precision=β₀+β₁×e^{(β}^{₂}^{×log}^{₁₀}^{(Observed Concentration))}+ε

where β₀, β₁, and β₂ are the coefficients of the model, and c is the random error term. JMP (Version 14.1, SAS Institute) was utilized for calculations with an exponential 3P fit curve model.

The Lower Limit of Quantitation (LLoQ) using the precision profile method was calculated as the concentration at the upper 95% confidence limit of the fitted curve at 20% CV.

The lower limit of quantification (LLoQ) for qPCR was determined to be 50 copies of viral genome/1 μg genomic DNA based on the criteria of≥95% detection, ≤20% variability and within 0.125 LOG of difference from the target value, exceeding the FDA guidance requirements.

The shedding assay detection range is summarized below. If the titer is detected to be above the upper quantitation limit, the sample was reported as ALQ (above limit of quantitation); if the titer is determined to be below the lower quantitation limit, the sample was reported as BLQ (below limit of quantitation); and if the titer is within the quantitation range, a numeric concentration was reported.

The raw signals for exemplary DMD AAV construct whole blood, saliva, stool and urine were determined using QuantStudio™ 7 Flex and QuantStudio™ Software v1.1. The concentrations of exemplary DMD AAV construct were calculated using a linear regression curve, which is generated by plotting Ct values of extracted exemplary DMD AAV construct viral calibrators in buffer against their known concentrations (log transformed). The concentration of exemplary DMD AAV construct viral DNA in samples is determined by fitting the Ct values in this model. This was done in Microsoft Excel, Microsoft Office 363 Pro version 1909. For tables, final concentrations were reported in viral genome (vg)/mL, except for stool where it was reported in vg/mg, to at least three significant figures. Precision (% CV) was reported to the nearest 0.1%.

Statistical Analysis

Accuracy—Titer data were considered log-normally distributed and analyzed following log 10 transformation. exemplary DMD AAV construct shedding assay accuracy was determined using the same panels described in the analytical sensitivity section below. Accuracy was calculated by the difference between observed value and the expected value after log transformation, and the target requirement was within ±0.5 log bias of expected value across the quantitative range. exemplary DMD AAV construct shedding assay linearity and accuracy for each sample type is summarized below.

Linearity—The assay linearity was assessed by fitting a linear regression model as follows

Log 10(Quantitation)=β0+β1×Log 10(Input Concentration)+ε

Where β0 is intercept, β1 is slope and c is the random error term. The coefficient of determination R² was calculated based on this model. Linearity is established if R² is above 0.95. Accuracy and linearity analysis was performed using R (Version 3.5.1 2018-07-02, https://cran.rproject.org/).

Precision—Assay within-laboratory reproducibility, or precision, was estimated using the following variance components model for each concentration level,

Quantitation=intercept+instrument+run+within-run (error)

Where instrument, run, and within-run errors were treated as random effects. Variance components were estimated via a random effect model. The total variance, δ², which accounted for all between-instrument, between-run, and within-run effects, was estimated as the sum of the individual variance components. The measure of reproducibility is the percent coefficient of variation (% CV), or the total variance divided by the mean. Variance components were analyzed in JMP (Version 13.1, SAS Institute) using REML (Restricted Maximum Likelihood) model (https://www.jmp.com/support/help/14-2/restricted-maximumlikelihood-reml-model.shtml).

Specificity—Assay specificity was calculated as the ratio of identified negative samples to all unspiked (true negative) samples. The assay specificity for exemplary DMD AAV construct shedding was determined with 20-24 biological replicates (20 for saliva, stool, and urine, and 24 for whole blood), using normal human saliva, stool, urine and whole blood samples without any viral spike. The specificity was higher than 95% in all sample type tested.

Analytical Sensitivity—For measurement of analytical sensitivity, the observed data were fitted with a nonlinear regression model as follows:

Precision=β₀+β₁×e\hat{ }((β₂×log 10(Observed Concentration)))+ε

where β0, β1, and β2 are the coefficients of the model, and c is the random error term. EVP (Version 14.1, SAS Institute) was utilized for calculations with an exponential 3P fit curve model.

The Lower Limit of Quantitation (LLoQ) using the precision profile method was calculated as the concentration at the upper 95% confidence limit of the fitted curve at 30% CV.

Analytical sensitivity was determined in a two-step process. In Step 1, for Saliva, Stool and Urine, an up to 12-replicate panel was tested on two different VERSANT kPCR extraction systems to estimate the Lower Limit of Quantitation (LLoQ), for Blood only one VERSANT kPCR extraction system was used to test at least 6 replicates each. In Step 2, up to 36 biological replicates near the estimated LLoQ level were utilized to further refine LLoQ for all sample types. The concentration at the upper 95% confidence limit of the fitted curve at 30% CV and within ±0.5 LOG of bias was determined as the LLoQ for that sample type. Exemplary DMD AAV construct shedding assay analytical sensitivity for each sample type are summarized below.

RESULTS

Shedding Assay Performance Summary—The shedding assay provided a quantitative result for saliva, stool, urine, and whole blood samples within corresponding assay ranges. The shedding assay detection range is summarized in Table 3. If the titer was detected to be above the upper quantitation limit, the sample was reported as AQL (above quantitation limit); if the titer was determined to be below the lower quantitation limit, the sample was be reported as BQL (below quantitation limit); and if the titer was within the quantitation range, a numeric concentration was reported.

TABLE 3
Performance Evaluation Summary

| | Lower Limit of | Upper Limit of |
|---|---|---|
| Matrix | Quantification (LLOQ) | Quantification (ULoQ) |
| Saliva | 3.67E+03 vg/mL | 9.84E+08 vg/mL |
| Stool | 3.90E+01 vg/mg | 1.28E+07 vg/mg |
| Urine | 4.26E+03 vg/mL | 1.14E+09 vg/mL |
| Whole Blood | 8.45E+03 vg/mL | 7.63E+08 vg/mL |

DNase Treatment Performance Summary—MNase treatment was chosen as nuclease for DNase treatment assessment. MNase is a DNA and RNA endonuclease and is able to cleave double-stranded DNA (dsDNA), single-stranded DNA (ssDNA) and RNA. MNase has higher activity than DNase I (MNase at 2,000,000 units/mL and DNase I at 2,000 units/mL are commercially available through New England Biolabs). Incomplete digestion of unprotected DNA was observed with DNase I (up to 250 units, limited by the stock concentration) in reaction buffer system. In comparison, treatment with 4,000 units of MNase eliminated the unprotected deoxynucleic acid (both dsDNA and ssDNA) and did not decrease exemplary Hem-B AAV construct recovery from intact viral particles, in all sample types tested (see immediate example 3).

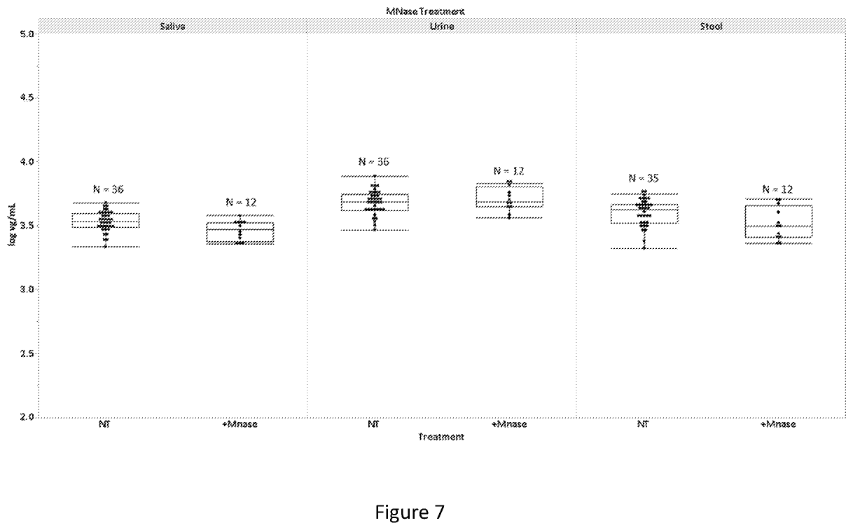

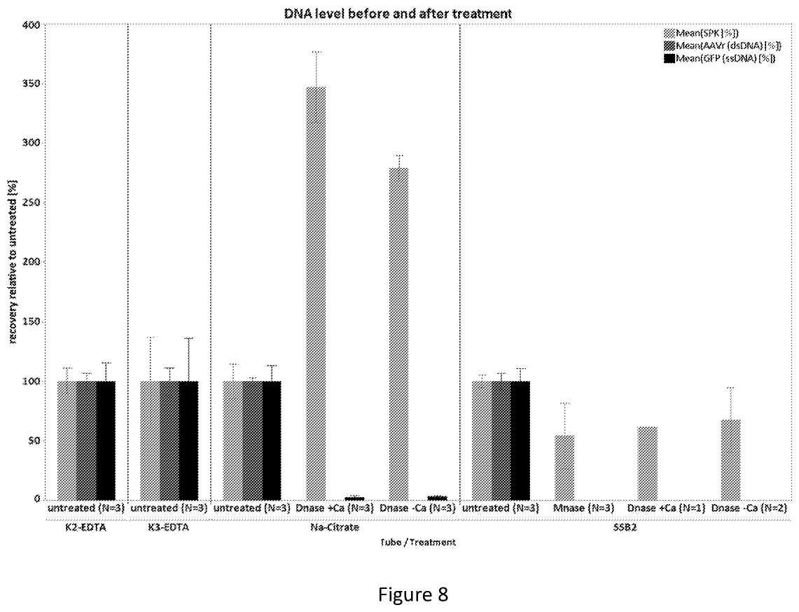

MNase treatment on intact exemplary DMD AAV construct viral DNA recovery was assessed in all sample types, except whole blood, at a concentration near the LLoQ, with a minimum of 12 replicates. No significant difference was observed between MNase treated or untreated samples for all sample types (see Table 4 below and FIG. 7). All the measured differences are within the assay accuracy of +/−0.5 log and the % changes are within an acceptable assay variation range. MNase treatment of linearized plasmid resulted in complete loss of DNA (CT undetermined). MNase treatment did not work for whole blood as a sample type as the MNase buffer coagulated the blood (see FIG. 8). DNase I did work for whole blood collected in sodium citrate collection tubes but had a strong negative effect on the already developed calibration system. Redevelopment of a calibration system for DNase I treated whole blood was not pursued as whole blood is not considered a regularly shed bodily fluid.

TABLE 4
MNase treatment summary

| | Untreated | MNase treated | Log | |
|---|---|---|---|---|
| Matrix | (vg/mL) N = 36 | (vg/mL) N = 12 | Difference | % Loss |
| Saliva | 3.45E+03 | 2.92E+03 | −0.073 | 15.39% |
| Urine | 4.85E+03 | 5.15E+03 | 0.026 | −6.10% |
| Stool | 4.06E+03 | 3.41E+03 | −0.076 | 16.05% |
| Linearize | 1.06E+05 | 0 | NA | 100% |
| plasmid | | | | |

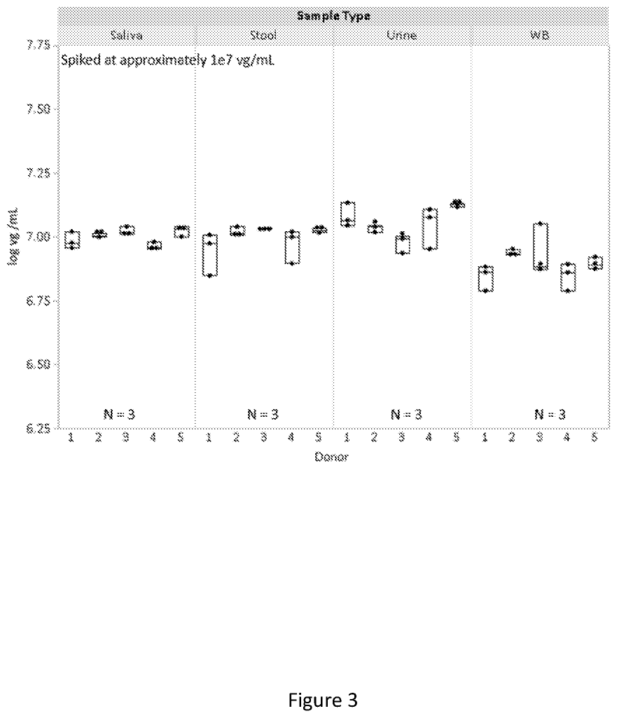

qPCR Performance—The FDA guidance requires qPCR to be able to detect<50 copies of vector/1 μg genomic DNA with 95% confidence. The qPCR quantitation limit was determined using a panel of linearized plasmid containing the exemplary DMD AAV construct viral genome spiked into human genomic DNA. N=444 was chosen for 25 copies of vector/1 μg genomic DNA to ensure that a 95% detection rate with enough confidence can be achieved (summarized in FIG. 3 and Tables 5 and 6)

TABLE 5
Analytical Sensitivity Results Summary (LloQ)

| | | | | | Accuracy | | LloQ |
|---|---|---|---|---|---|---|---|
| | | | | | (LogObs- | | [95% |
| Expected | Observed | No. | | | LogExp, | | CI] |
| (vg/1 μg | (vg/1 μg | of | No. | Detection | Within ± | Precision | (vg/1 μg |
| gDNA) | gDNA) | Reps | Detected | Rate | 0.125LOG) | (<20% CV) | gDNA) |
| 100 | 99.7 | 12 | 12 | 100.0% | −0.0013 | 11.93% | 50 |
| 75 | 39.0 | 12 | 12 | 100.0% | −0.0059 | 16.08% | |
| 50 | 52.2 | 16 | 16 | 100.0% | 0.0187 | 17.17% | |
| 37 | 39.0 | 12 | 12 | 100.0% | 0.0467 | 20.52% | |
| 25 | 23.8 | 12 | 12 | 100.0% | −0.0208 | 32.20% | |
| 20 | 22.0 | 12 | 12 | 100.0% | 0.0406 | 29.59% | |
| 10 | 10.7 | 12 | 12 | 100.0% | 0.0291 | 42.67% | |

TABLE 6
qPCR Limit of Detection

| Expected | Observed | No. of | No. | No. of | Detection |
|---|---|---|---|---|---|
| (vg/1 μg gDNA) | (vg/1 μg gDNA) | Reps | Detected | Runs | Rate |
| 25 | 27.2 | 444 | 444 | 3 | 100.0% |

Accuracy—Titer data were considered to be log-normally distributed and were analyzed following log 10 transformation. Exemplary DMD AAV construct shedding assay accuracy was determined using the same sample panels described above for analytical sensitivity. Accuracy was calculated by the difference between observed value and the expected value after log transformation, and the target requirement was with ±0.5 log bias of expected value across the quantitative range. Exemplary DMD AAV construct shedding assay linearity and accuracy for each sample type was summarized and is presented in Tables 7-10.

TABLE 7
Assessment of Accuracy of Shedding Assay of Exemplary
DMD AAV Construct in Saliva

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 9.8E+08 | 8.99 | 8.99 | 12 | 0.01 |
| 9.6E+06 | 6.97 | 6.98 | 12 | 0.01 |
| 1.0E+05 | 4.96 | 5.00 | 12 | 0.04 |
| 1.2E+04 | 4.12 | 4.07 | 10 | −0.06 |
| 9.2E+03 | 3.95 | 3.96 | 12 | 0.01 |
| 6.5E+03 | 3.78 | 3.81 | 36 | 0.04 |
| 6.3E+03 | 3.79 | 3.80 | 12 | 0.00 |
| 5.0E+03 | 3.73 | 3.70 | 12 | −0.03 |
| 3.8E+03 | 3.55 | 3.58 | 12 | 0.03 |
| 3.4E+03 | 3.53 | 3.54 | 36 | 0.01 |
| 1.8E+03 | 3.27 | 3.24 | 36 | −0.03 |

TABLE 8
Assessment of Accuracy of Shedding Assay of Exemplary DMD AAV
Construct in Stool*

| | Observed | Expected | | |
|---|---|---|---|---|
| Concentration | LogQTY | LogQTY | No. of | Log Difference |
| (vg/mg) | (Log vg/mg) | (Log vg/mg) | Reps | (LogObs-LogExp) |
| 1.28E+07 | 6.99 | 7.11 | 11 | 0.12 |
| 1.40E+05 | 4.98 | 5.15 | 12 | 0.17 |
| 1.35E+03 | 2.97 | 3.13 | 12 | 0.16 |
| 1.29E+02 | 1.97 | 2.11 | 10 | 0.14 |
| 9.86E+01 | 1.95 | 1.99 | 35 | 0.05 |
| 6.70E+01 | 1.67 | 1.83 | 12 | 0.16 |
| 4.91E+01 | 1.62 | 1.69 | 36 | 0.07 |
| 4.25E+01 | 1.57 | 1.63 | 11 | 0.06 |
| 4.06E+01 | 1.53 | 1.61 | 36 | 0.08 |
| 3.63E+01 | 1.51 | 1.56 | 35 | 0.05 |
| 3.07E+01 | 1.37 | 1.49 | 12 | 0.12 |
| 2.08E+01 | 1.15 | 1.32 | 12 | 0.17 |

*Stool was suspended in 10 volume (w/v) of 1X PBS before extraction.

TABLE 9
Assessment of Accuracy of Shedding Assay of Exemplary DMD AAV
Construct in Urine

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 1.1E+09 | 9.00 | 9.06 | 12 | 0.06 |
| 1.2E+07 | 6.98 | 7.07 | 12 | 0.08 |
| 1.1E+05 | 4.98 | 5.05 | 12 | 0.07 |
| 1.8E+04 | 4.15 | 4.26 | 12 | 0.11 |
| 1.2E+04 | 3.97 | 4.07 | 12 | 0.10 |
| 8.7E+03 | 3.88 | 3.94 | 12 | 0.06 |
| 6.6E+03 | 3.75 | 3.82 | 12 | 0.07 |
| 4.9E+03 | 3.67 | 3.69 | 36 | 0.01 |
| 4.1E+03 | 3.58 | 3.61 | 11 | 0.03 |
| 3.9E+03 | 3.63 | 3.59 | 36 | −0.03 |
| 3.3E+03 | 3.55 | 3.51 | 36 | −0.04 |

TABLE 10
Assessment of Accuracy of Shedding Assay of Exemplary
DMD AAV Construct in Whole Blood

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 7.63E+08 | 9.00 | 8.88 | 6 | −0.12 |
| 7.80E+06 | 7.01 | 6.89 | 8 | −0.11 |
| 1.25E+05 | 5.03 | 5.10 | 36 | 0.07 |
| 8.95E+04 | 5.00 | 4.95 | 9 | −0.05 |
| 1.67E+04 | 4.21 | 4.22 | 36 | 0.02 |
| 1.32E+04 | 4.19 | 4.12 | 9 | −0.07 |
| 9.93E+03 | 4.03 | 4.00 | 35 | −0.04 |
| 7.89E+03 | 4.01 | 3.90 | 9 | −0.12 |
| 6.70E+03 | 3.92 | 3.83 | 9 | −0.09 |
| 6.47E+03 | 3.94 | 3.81 | 35 | −0.12 |
| 3.68E+03 | 3.79 | 3.57 | 8 | −0.23 |
| 2.26E+03 | 3.62 | 3.35 | 8 | −0.26 |

Specificity—Assay specificity was calculated as the ratio of identified negative samples to all unspiked (true negative) samples. The assay specificity for exemplary DMD AAV construct shedding was determined with 20-24 biological replicates (20 for saliva, stool, and urine, and 24 for whole blood), using normal human saliva, stool, urine and whole blood samples without any viral spike. The specificity was higher than 95% in all sample type tested.

Analytical Sensitivity—Analytical sensitivity was determined in a two-step process. In Step 1, for Saliva, Stool and Urine, an up to 12-replicate panel was tested on two different VERSANT kPCR extraction systems to estimate the Lower Limit of Quantitation (LLoQ), for Blood only one VERSANT kPCR extraction system was used to test at least 6 replicates each. In Step 2, up to 36 biological replicates near the estimated LLoQ level were utilized to further refine LLoQ for all sample types. The concentration at the upper 95% confidence limit of the fitted curve at 30% CV and within ±0.5 LOG of bias was determined as the LLoQ for that sample type. Exemplary DMD AAV construct shedding assay analytical sensitivity for each sample type are summarized below in Tables 11-14.

TABLE 11
Analytical Sensitivity and Specificity for Exemplary DMD
AAV Construct in Human Saliva

| Concen- | No. | | | | | LloQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 9.84E+08 | 12 | 12 | 100.0% | 0.006 | 10.61% | 3670 |
| 9.58E+06 | 12 | 12 | 100.0% | 0.012 | 7.67% | |
| 9.98E+04 | 12 | 12 | 100.0% | 0.040 | 12.11% | |
| 1.17E+04 | 12 | 10 | 100.0% | −0.059 | 17.61% | |
| 9.17E+03 | 12 | 12 | 100.0% | 0.014 | 16.78% | |
| 6.53E+03 | 36 | 36 | 100.0% | 0.036 | 16.84% | |
| 6.27E+03 | 12 | 12 | 100.0% | 0.002 | 26.80% | |
| 5.01E+03 | 12 | 12 | 100.0% | −0.026 | 18.81% | |
| 3.81E+03 | 12 | 12 | 100.0% | 0.032 | 29.93% | |
| 3.44E+03 | 36 | 36 | 100.0% | 0.008 | 17.50% | |
| 1.75E+03 | 36 | 36 | 100.0% | −0.030 | 30.19% | |

TABLE 12
Analytical Sensitivity and Specificity for Exemplary DMD
AAV Construct in Human Stool*

| Concen- | No. | | | | | LloQ |
|---|---|---|---|---|---|---|
| tration | of | No. | Detection | (LogObs- | Total | [95% CI] |
| (vg/mg) | Reps | Detected | Rate | LogExp) | % CV | (vg/mg) |
| 1.28E+07 | 11 | 11 | 100.0% | 0.122 | 19.52% | 39 |
| 1.40E+05 | 12 | 12 | 100.0% | 0.168 | 16.55% | |
| 1.35E+03 | 12 | 12 | 100.0% | 0.158 | 9.47% | |
| 1.29E+02 | 12 | 10 | 100.0% | 0.139 | 16.48% | |
| 9.86E+01 | 36 | 35 | 100.0% | 0.047 | 20.16% | |
| 6.70E+01 | 12 | 12 | 100.0% | 0.158 | 18.56% | |
| 4.91E+01 | 36 | 36 | 100.0% | 0.069 | 19.30% | |
| 4.25E+01 | 12 | 11 | 100.0% | 0.057 | 25.61% | |
| 4.06E+01 | 36 | 36 | 100.0% | 0.081 | 21.48% | |
| 3.63E+01 | 36 | 35 | 100.0% | 0.045 | 21.99% | |
| 3.07E+01 | 12 | 12 | 100.0% | 0.118 | 43.82% | |
| 2.08E+01 | 12 | 12 | 100.0% | 0.172 | 39.11% | |

*Stool was suspended in 10 volume (w/v) of 1X PBS before extraction.

TABLE 13
Analytical Sensitivity and Specificity for Exemplary DMD
AAV Construct in Human Urine

| Concen- | No. | | | | | LloQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 1.14E+09 | 12 | 12 | 100.0% | 0.061 | 16.02% | 4263 |
| 1.17E+07 | 12 | 12 | 100.0% | 0.083 | 17.90% | |
| 1.12E+05 | 12 | 12 | 100.0% | 0.070 | 14.03% | |
| 1.83E+04 | 12 | 12 | 100.0% | 0.110 | 13.88% | |
| 1.17E+04 | 12 | 12 | 100.0% | 0.095 | 17.88% | |
| 8.67E+03 | 12 | 12 | 100.0% | 0.061 | 19.96% | |
| 6.62E+03 | 12 | 12 | 100.0% | 0.069 | 14.13% | |
| 4.85E+03 | 36 | 36 | 100.0% | 0.013 | 20.85% | |
| 4.05E+03 | 12 | 11 | 100.0% | 0.030 | 33.65% | |
| 3.92E+03 | 36 | 36 | 100.0% | −0.034 | 25.13% | |
| 3.25E+03 | 36 | 36 | 100.0% | −0.036 | 26.56% | |

TABLE 14
Analytical Sensitivity and Specificity for Exemplary DMD
AAV Construct in Human Whole Blood

| Concen- | No. | | | | | LloQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 7.63E+08 | 6 | 6 | 100.0% | −0.121 | 14.94% | 8451 |
| 7.80E+06 | 9 | 8 | 100.0% | −0.114 | 21.68% | |
| 1.25E+05 | 36 | 36 | 100.0% | 0.074 | 20.01% | |
| 8.95E+04 | 9 | 9 | 100.0% | −0.051 | 22.74% | |
| 1.67E+04 | 36 | 36 | 100.0% | 0.015 | 20.81% | |
| 1.32E+04 | 9 | 9 | 100.0% | −0.066 | 18.70% | |
| 9.93E+03 | 36 | 35 | 100.0% | −0.037 | 28.45% | |
| 7.89E+03 | 9 | 9 | 100.0% | −0.117 | 22.61% | |
| 6.70E+03 | 9 | 9 | 100.0% | −0.090 | 34.12% | |
| 6.47E+03 | 36 | 35 | 100.0% | −0.124 | 28.98% | |
| 3.68E+03 | 9 | 8 | 100.0% | −0.225 | 40.85% | |
| 2.26E+03 | 9 | 8 | 100.0% | −0.264 | 43.47% | |

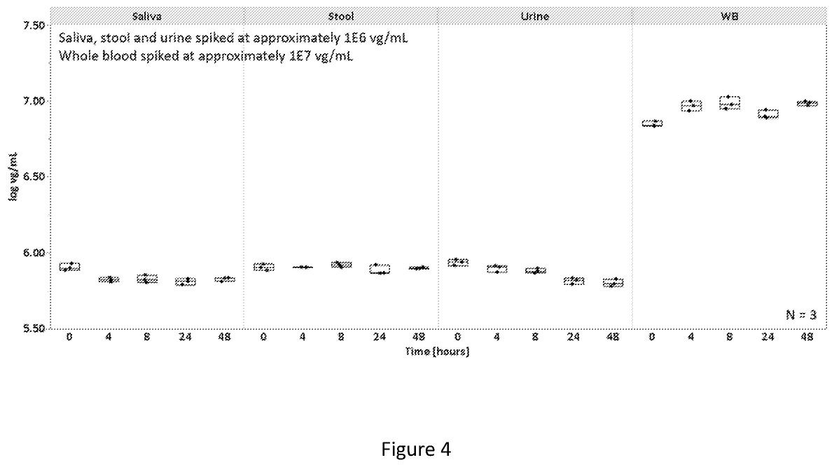

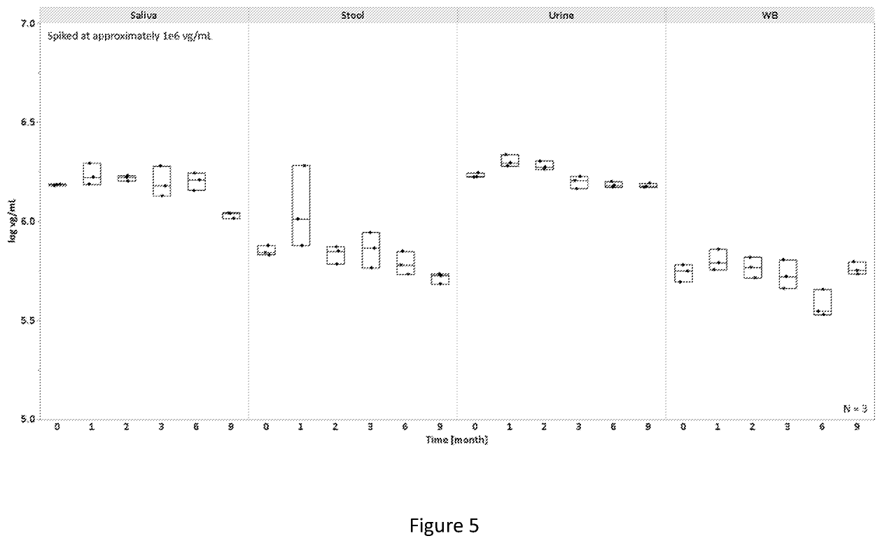

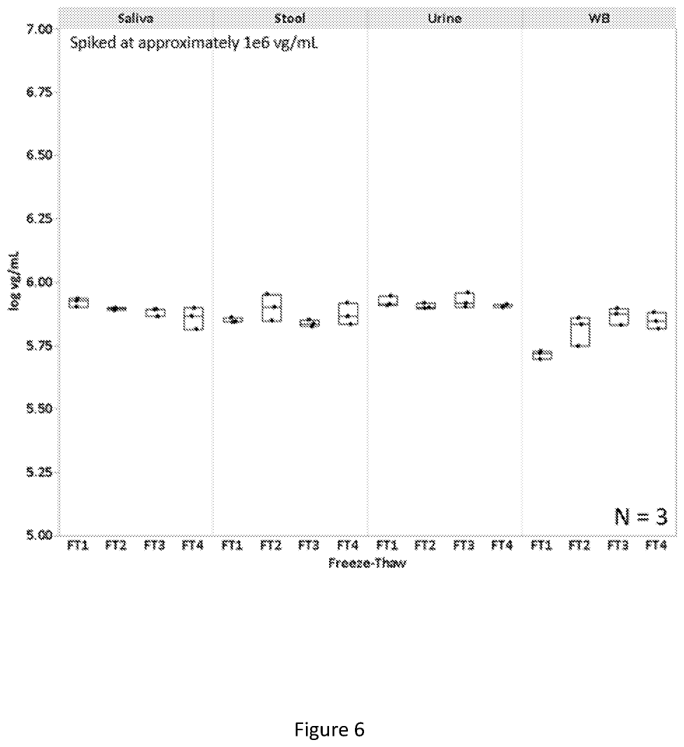

Ambient temperature, Repeated Freeze/Thaw, and Long-term −80° C. Storage Stability of PBMCs, Saliva, Urine and Stool—Stability of exemplary DMD AAV construct virus in PBMCs, saliva, stool and urine stored at ambient temperature, long term storage at −80° C. for up to 6 months, and freeze/thaw cycling was assessed. Pooled samples were spiked at 1.0E+05 vg/mL (post 5E+6 cells/mL suspension of PBMCs and 1:10 (w:v) suspension of stool), and three biological replicates per condition were tested at indicated time points. Exemplary DMD AAV construct viral genome titer was within ±0.5 LOG of the titer determined at time 0 for up to 6 months. Long-term −80° C. Storage Stability results are summary in FIG. 5 and Table 15. Ambient temperature stability is summarized in FIG. 4. Freeze/Thaw assessments are summarized in FIG. 6.

TABLE 15
Long Term Stability of Exemplary DMD AAV
Construct Stored and Frozen at −80° C.

| | | | Log Mean | Log Mean | | |
|---|---|---|---|---|---|---|
| | Timepoint | No. of | Titer [log | Diff from | CV | Recovery |
| Matrix | (months) | Replicates | vg/mL] | T0 | [%] | [%] |
| Saliva | 0 | 3 | 6.20 | NA | 1.43 | NA |
| | 1 | 3 | 6.02 | −0.12 | 3.77 | 75.90 |
| | 2 | 3 | 6.17 | −0.03 | 3.62 | 93.44 |
| | 3 | 3 | 6.10 | −0.10 | 1.64 | 79.46 |
| | 6 | 3 | 6.20 | 0.01 | 9.99 | 101.39 |
| | 9 | 3 | 6.03 | −0.17 | 3.36 | 68.29 |
| Stool | 0 | 3 | 5.85 | NA | 5.66 | NA |
| | 1 | 3 | 5.86 | 0.01 | 38.87 | 103.46 |
| | 2 | 3 | 5.81 | −0.04 | 9.64 | 91.11 |
| | 3 | 3 | 5.81 | −0.04 | 9.89 | 91.09 |
| | 6 | 3 | 5.79 | −0.06 | 13.63 | 87.68 |
| | 9 | 3 | 5.72 | −0.13 | 6.12 | 73.84 |
| Urine | 0 | 3 | 6.24 | NA | 0.80 | NA |
| | 1 | 3 | 6.20 | −0.03 | 0.29 | 92.95 |
| | 2 | 3 | 6.26 | 0.02 | 1.11 | 105.56 |
| | 3 | 3 | 6.16 | −0.07 | 3.53 | 84.58 |
| | 6 | 3 | 6.19 | −0.05 | 3.50 | 89.03 |
| | 9 | 3 | 6.18 | −0.05 | 2.45 | 88.11 |
| Whole | 0 | 3 | 5.72 | NA | 3.68 | NA |
| Blood | 1 | 3 | 5.72 | 0.00 | 11.22 | 100.05 |
| | 2 | 3 | 5.77 | 0.05 | 4.61 | 112.44 |
| | 3 | 3 | 5.73 | 0.01 | 13.01 | 103.47 |
| | 6 | 3 | 5.58 | −0.13 | 16.61 | 73.59 |
| | 9 | 3 | 5.76 | 0.05 | 7.55 | 111.52 |

### EXAMPLE 3: Identifying and Optimizing Primers for Exemplary Hemophilia B AAV Gene Therapy Shedding Quantification

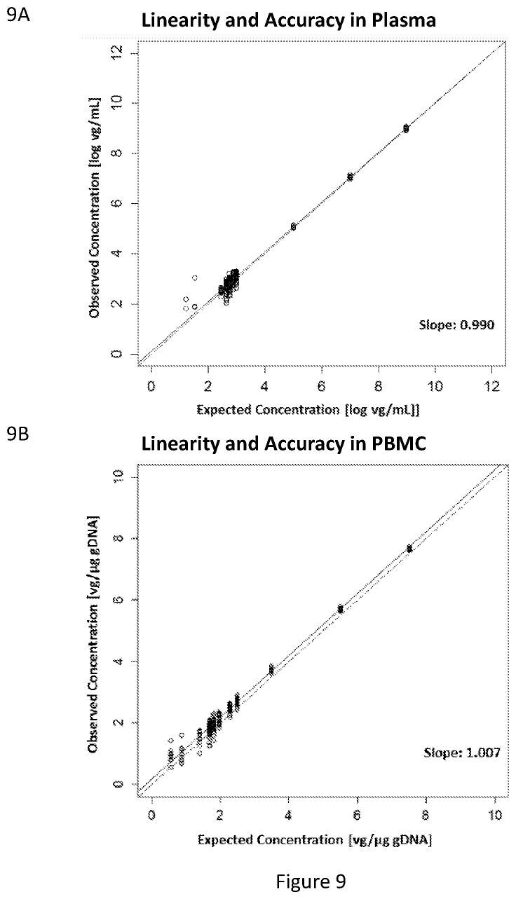

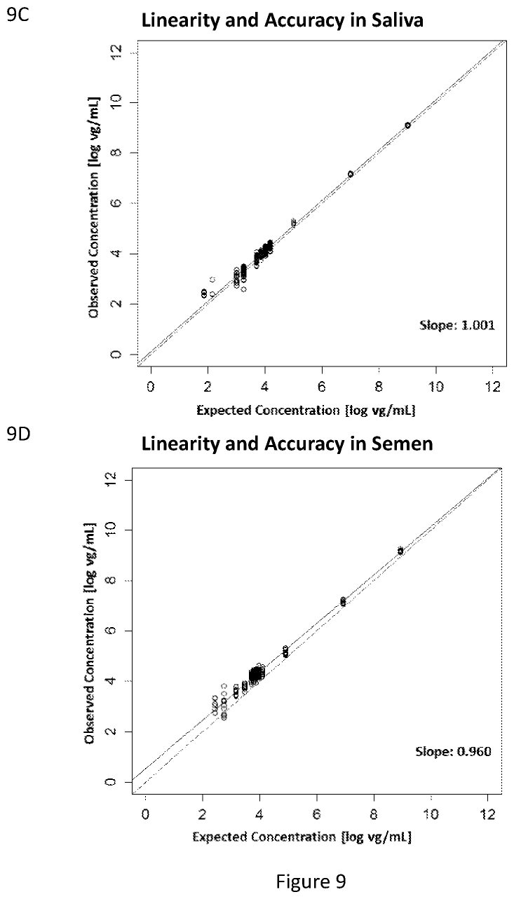

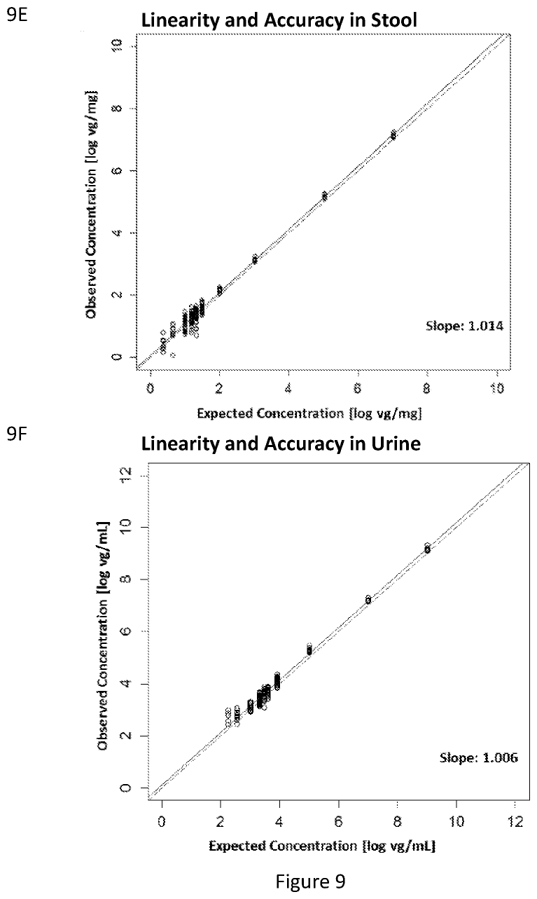

The present example demonstrates the utility, accuracy, sensitivity, and precision of assays performed utilizing oligonucleotide sequences described herein. This shedding assay detects and quantifies an exemplary viral DNA (e.g., exemplary Hem-B AAV construct) extracted from urine, plasma, semen, whole blood, stool and saliva. Exemplary Hem-B AAV construct is an adeno-associated virus encoding factor IX. Extracted DNA was tested in a qPCR reaction using 5 μL of DNA extract. A linear regression curve was generated by plotting Ct values of extracted exemplary Hem-B AAV construct viral calibrators in buffer against their known concentrations (log transformed, see FIG. 9). The concentration of exemplary Hem-B AAV construct viral DNA in samples was determined by fitting their Ct values in this model. The concentration of exemplary Hem-B AAV construct viral DNA in urine, plasma, semen, whole blood, and saliva samples were reported as viral genomes (vg)/mL and stool samples were reported as vg/mg.

The FDA guidance requires qPCR to be able to detect<50 copies of vector/1 μg genomic DNA with 95% confidence. The qPCR quantitation limit was determined using a panel of linearized plasmid containing the exemplary Hem-B AAV construct viral genome spiked into human genomic DNA. Linearized plasmid at different concentrations was tested with different numbers of replicates to assess the quantification limits. N=75 was chosen for the lowest concentration to ensure a 95% detection rate with enough confidence. The lower limit of quantification (LLoQ) for qPCR was determined to be 31 copies of viral genome/1 μg genomic DNA based on the criteria of ≥95% detection, <20% variability and within 0.125 LOG of difference from the target value (summarized below).

The present example demonstrates the utility, accuracy, sensitivity, and precision of assays performed utilizing oligonucleotide sequences described herein.

Primary Screening of Exemplary Primers and/or Probes

Primers were designed targeting select regions of an exemplary HemB AAV construct (see FIG. 1). Prior to complete oligonucleotide characterization, at least one or more forward primer, at least one or more reverse primer, and at least one or more probes were screened for characteristics such as multiplexing capacity, maximum fluorescence, slope, minimum CT, Tm, Oligonucleotide cross reactivity, and/or specificity for HemB AAV constructs in the presence of background genomic DNA.

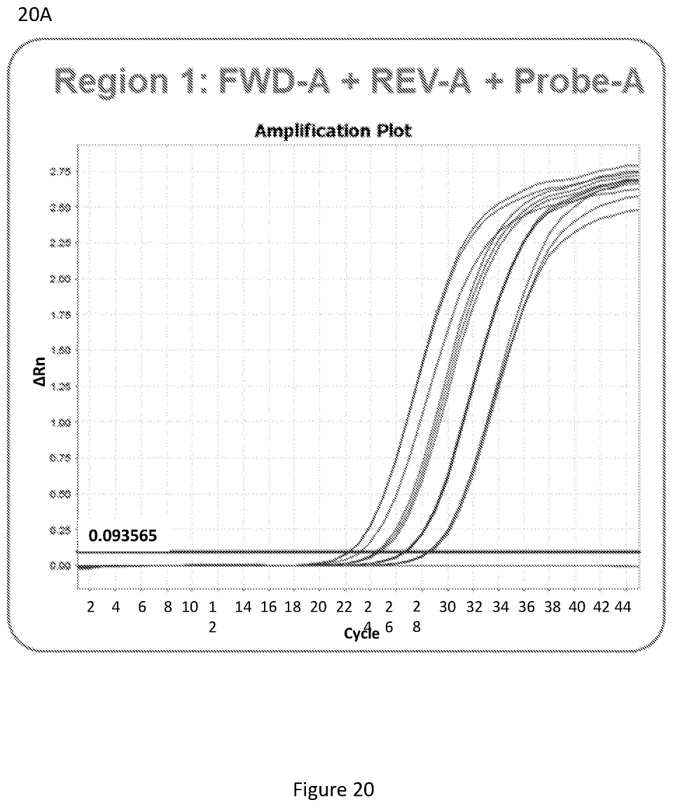

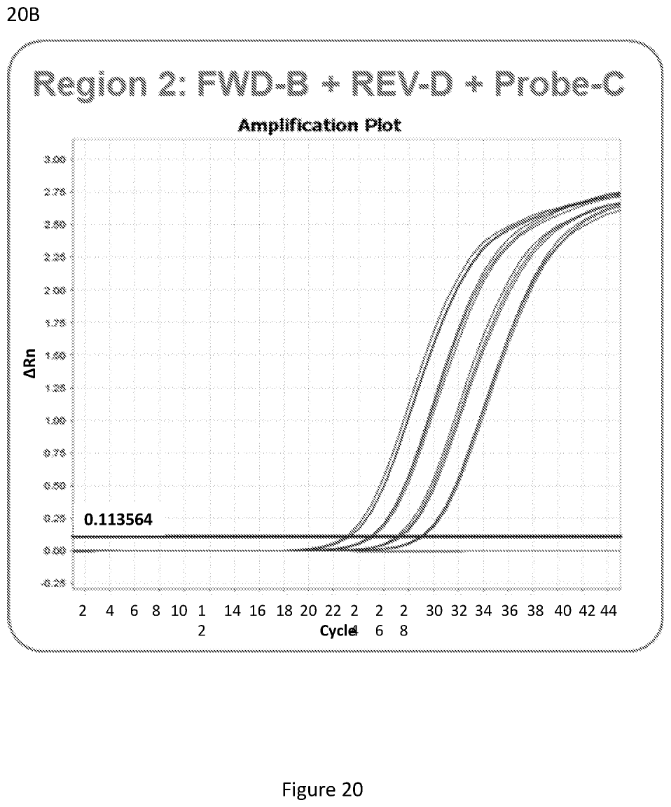

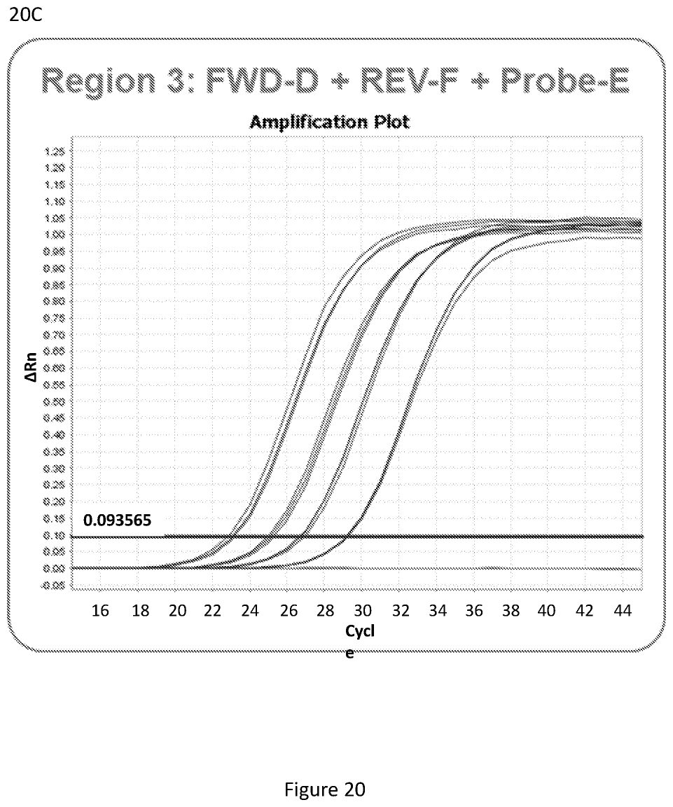

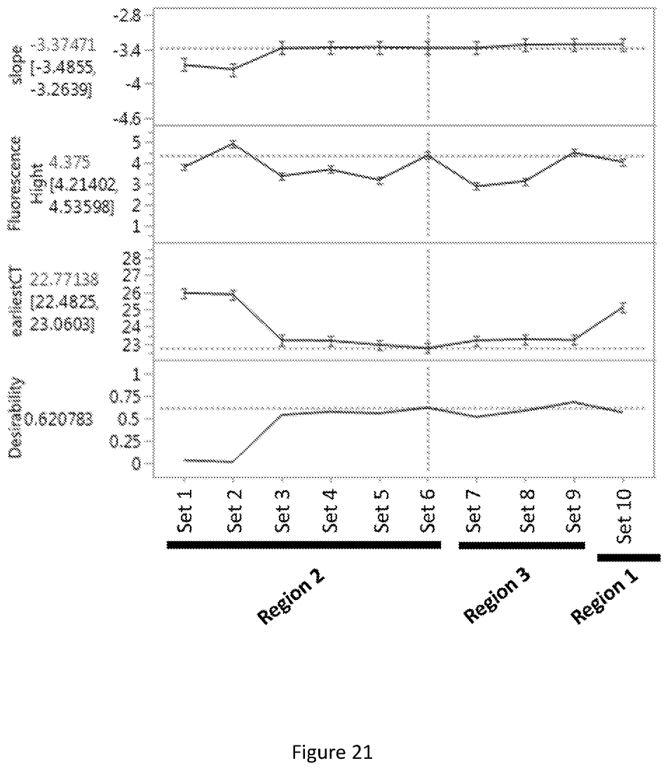

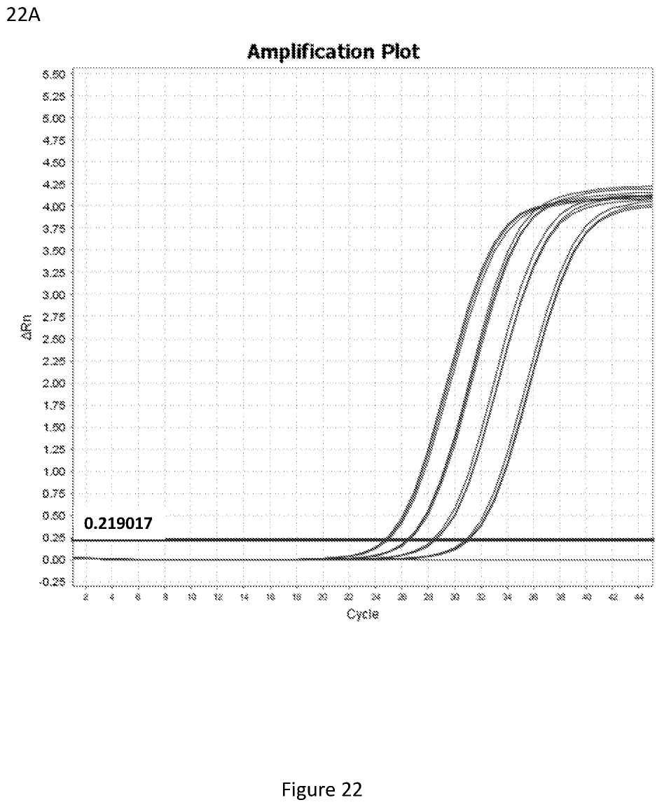

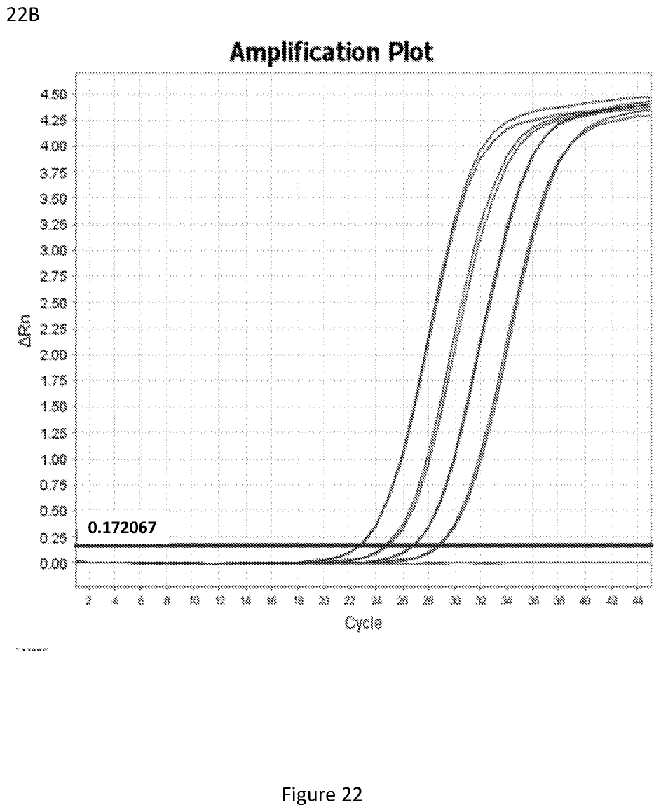

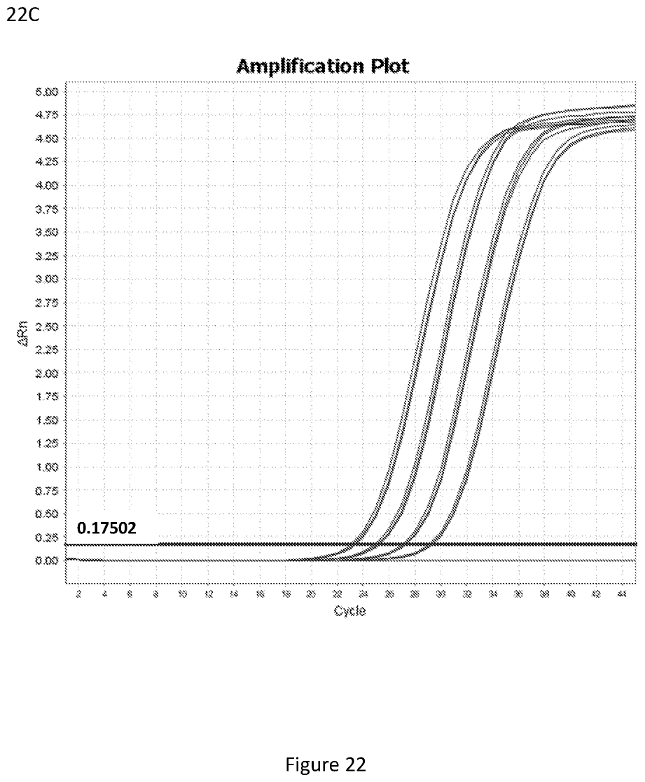

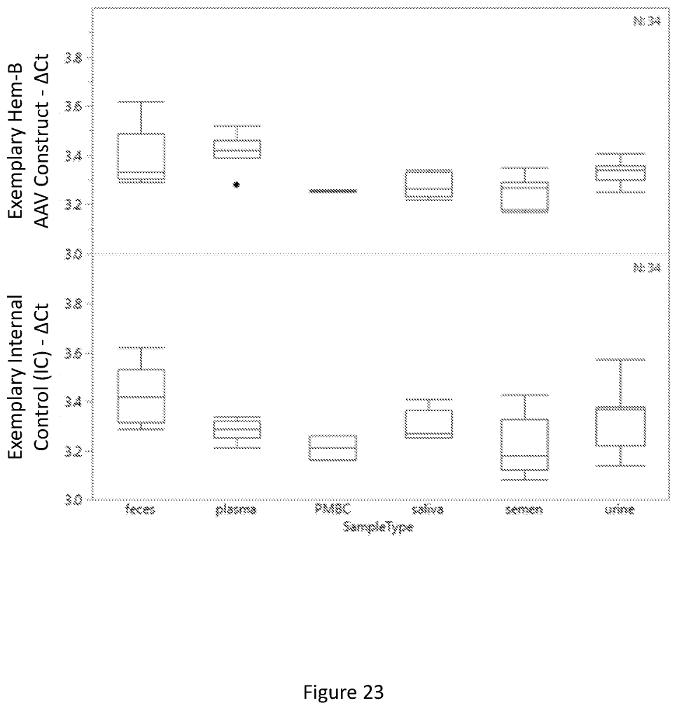

Certain primer and probe combinations were determined to work well under tested assay conditions. As shown in the figures and further herein, certain primer and probe combinations provided a desirable profile. However, alternative primer and/or probe combinations can be screened as shown to determine desirable profiles for other conditions, samples, etc., and the present disclosure recognizes that multiple primer and/or probe combinations may be suitable for accurate and reliable detection and/or quantification. The data also confirms that select primer and probe combinations work well when multiplexed, for example, with internal control primer and probe combinations. In some embodiments, those of skill in the art will understand that certain characteristics may be more or less desirable and that selection of a set of primers and probes for a particular application may be dependent on the weighing of certain characteristics such as multiplexing capacity, maximum fluorescence, slope, minimum CT, Tm, oligonucleotide cross reactivity, specificity for HemB AAV constructs in the presence of background genomic DNA, or any combination of all or any of these factors. Nonetheless, the data provided herein established that the disclosed primers and/or probes worked well for the detection of AAV constructs in biological samples. At least one or more forward primers, at least one or more reverse primers, and at least one or more probes were found suitable for one or more primary screening criteria (for certain results see e.g., Table 16-18, and FIGS. 20-23).

TABLE 16
Primary Screening of Certain Oligonucleotide
Combinations targeting alternative Exemplary
HEM-B AAV Construct regions.

| | Exem- | | | | | |
|---|---|---|---|---|---|---|
| | plary | | | | | |
| | Hem- | | | | | |
| | BAAV | | | | | |
| | Con- | | | | Amplicon | |
| | struct | | | Detec- | Size | |
| Assay | Region | Fwd-P | Rev-P | tion | [bp] | |
| 1 | Region | HemB- | HemB- | HemB- | 81 | |
| | 1 | FWD-A | REV-A | Probe-A | | |
| | | (SEQ ID | (SEQ ID | (SEQ ID | | |
| | | NO: 33) | NO: 39) | NO: 47) | | |
| 2 | Region | HemB- | HemB- | HemB- | 107 | |
| | 2 | FWD-B | REV-C | Probe-B | | |
| | | (SEQ ID | (SEQ ID | (SEQ ID | | |
| | | NO: 35) | NO: 43) | NO: 51) | | |
| 3 | | HemB- | HemB- | HemB- | 82 | |
| | | FWD-C | REV-B | Probe-D | | |
| | | (SEQ ID | (SEQID | (SEQ ID | | |
| | | NO: 37) | NO: 41) | NO: 59) | | |
| 4 | | HemB- | HemB- | HemB- | 101 | |
| | | FWD-B | REV-D | Probe-C | | |
| | | (SEQ ID | (SEQ ID | (SEQ ID | | |
| | | NO: 35) | NO: 45) | NO: 55) | | |
| 5 | Region | HemB- | HemB- | HemB- | 140 | |
| | 3 | FWD-D | REV-F | Probe-E | | |
| | | (SEQ ID | (SEQ ID | (SEQ ID | | |
| | | NO: 81) | NO: 87) | NO: 89) | | |
| | | | | | | Ampl. |
| | | | | | | effi- |
| | 62500 | 15625 | 3906.25 | 976.56 | | cien- |
| Assay | copies | copies | copies | copies | slope | cy |
| 1 | 22.6 | 24.6 | 26.7 | 28.6 | −3.32 | 100.1 |
| 2 | 23.1 | 25.2 | 27.2 | 29.1 | −3.31 | 100.5 |
| 3 | 24.1 | 26.1 | 28.2 | 30.1 | −3.35 | 98.9 |
| 4 | 23.2 | 25.1 | 27.3 | 29.2 | −3.33 | 99.8 |
| 5 | 23.0 | 25.1 | 26.8 | 29.2 | −3.36 | 98.6 |

TABLE 17
Primary Screening of Certain Multiplexed Assay Results of Exemplary
HEM-B AAV Constructs

| Assay Set | DNA Source | Fluorescence Channel | Assay Primers Target |
|---|---|---|---|
| 1 | Exemplary HemB AAV | HemB AAV probe specific | Exemplary HemB AAV |
| | Construct | | Construct |
| 2 | IC-dsDNA | IC probe specific | IC target 3 |
| 3 | Exemplary HemB AAV | HemB AAV probe specific | Exemplary HemB AAV |
| 4 | Construct and | IC probe specific | Construct and IC target |
| | IC-dsDNA | | 3 |
| 5 | Exemplary HemB AAV | HemB AAV probe specific | Exemplary HemB AAV |
| 6 | Construct | IC probe specific | Construct and IC target |
| 7 | Exemplary HemB AAV | HemB AAV probe specific | 3 |
| 8 | Construct | IC probe specific | |
| 9 | Exemplary HemB AAV | HemB AAV probe specific | |
| 10 | Construct and | IC probe specific | |
| | IC-dsDNA | | |

| | 62500 | 15625 | 3906.25 | 976.56 | 244.1 | | Ampl. |
|---|---|---|---|---|---|---|---|
| Assay Set | copies | copies | copies | copies | copies | slope | efficiency |
| 1 | 22.4 | 24.5 | 26.9 | 29.0 | 30.6 | −3.5 | 94.1 |
| 2 | 19.2 | 21.2 | 23.3 | 25.3 | 27.5 | −3.4 | 96.5 |
| 3 | 22.1 | 24.2 | 26.4 | 28.4 | 30.5 | −3.5 | 93.3 |
| 4 | 19.0 | 21.1 | 23.2 | 25.2 | 27.2 | −3.4 | 97.0 |
| 5 | 22.0 | 24.1 | 26.4 | 28.5 | 30.3 | −3.5 | 93.9 |
| 6 | N/A | N/A | N/A | N/A | N/A | N/A | N/A |
| 7 | N/A | N/A | N/A | N/A | N/A | N/A | N/A |
| 8 | 19.6 | 21.5 | 23.5 | 25.5 | 27.4 | −3.3 | 102.2 |
| 9 | 22.1 | 24.3 | 25.8 | 28.5 | 30.5 | −3.5 | 94.6 |
| 10 | 19.5 | 21.5 | 23.5 | 25.4 | 27.7 | −3.4 | 98.7 |

TABLE 18
Certain Primer/Probe Sets for Amplifying and Quantifying
Exemplary Hem-B AAV Constructs

| Exemplary | | | | | |
|---|---|---|---|---|---|
| Hem-B AAV | | | | | |
| Construct | Assay | | | | |
| Region | Primer/Probe Set | Slope | Fluorescence | CT | N |
| Region 1 | Set 10 | −3.2 | 4.0 | 24.8 | 10 |
| | (SEQ ID NOs: 33, | | | | |
| | 39, and 47) | | | | |
| Region 2 | Set 6 | −3.37 | 4.4 | 22.8 | 10 |
| | (SEQ ID NOs: 35, | | | | |
| | 45, and 55) | | | | |
| Region 3 | Set 9 | −3.31 | 4.5 | 23.2 | 10 |
| | (SEQ ID NOs: 81, | | | | |
| | 87, and 89) | | | | |

Statistical Analysis

Accuracy—Assay accuracy was determined by calculating the average log recovery, defined as the average difference between the observed mean log quantitation and the log input concentration for levels tested (expected).

Linearity—The assay linearity was assessed by fitting a linear regression model as follows

Log 10(Quantitation)=β0+β1×Log 10(Input Concentration)+ε

Where β0 is intercept, β1 is slope and c is the random error term. The coefficient of determination R² was calculated based on this model. Linearity is established if R² is above 0.95. Accuracy and linearity analysis was performed using R (Version 3.5.1 2018-07-02, https://cran.rproject.org/)

Precision—Assay within-laboratory reproducibility, or precision, was estimated using the following variance components model for each concentration level,

Quantitation=intercept+instrument+run+within-run (error)

Where instrument, run, and within-run errors were treated as random effects. Variance components were estimated via a random effect model. The total variance, δ², which accounted for all between-instrument, between-run, and within-run effects, was estimated as the sum of the individual variance components. The measure of reproducibility is the percent coefficient of variation (% CV), or the total variance divided by the mean. Variance components were analyzed in JMP (Version 13.1, SAS Institute) using REML (Restricted Maximum Likelihood) model (https://www.jmp.com/support/help/14-2/restricted-maximumlikelihood-reml-model.shtml).

Specificity—Assay specificity was calculated as one minus the ratio of false positive samples to all unspiked (negative) samples.

Analytical Sensitivity—The Lower Limit of Quantitation (LLoQ) is the lowest input concentration that meets all above criteria and at which detection is at least 95%.

RESULTS:

qPCR Performance Summary—The lower limit of quantitation (LLoQ) of qPCR is 31 copies of exemplary Hem-B AAV construct viral DNA in the background of 1 μg genomic DNA.

Shedding Assay Performance Summary—The shedding assay provides a quantitative result for plasma, PBMC, saliva, semen, stool and urine samples within corresponding assay ranges. The shedding assay detection range is summarized in Table 19. If the titer was detected to be above the upper quantitation limit, the sample was reported as AQL (above quantitation limit); if the titer was determined to be below the lower quantitation limit, the sample was be reported as BQL (below quantitation limit); and if the titer was within the quantitation range, a numeric concentration was reported.

TABLE 19
Shedding assay detection range summary

| Sample | Lower Limit of Quantitation | Upper Limit of Quantitation |
|---|---|---|
| Type | (LLoQ) | (ULoQ) |
| Plasma | 1440 vg/mL | 1.0E+08 vg/mL |
| PBMC | 316 vg/1 μg genomic DNA | 4.7E+07 vg/1 μg genomic DNA |
| Saliva | 6172 vg/mL | 1.3E+09 vg/mL |
| Semen | 6037 vg/mL | 1.5E+09 vg/mL |
| Stool | 25 vg/mg | 1.4E+07 vg/mg stool |
| Urine | 5494 vg/mL | 1.4E+09 vg/mL |

DNase Treatment Performance Summary—MNase was the chosen nuclease for the DNase treatment. MNase acts as a potent DNA and RNA endonuclease, cleaving double-stranded DNA(dsDNA), single-stranded DNA(ssDNA) and RNA. MNase is known to be more potent than DNase I (MNase at 2,000,000 units/mL and DNase I at 2,000 units/mL are commercially available through New England Biolabs). Incomplete digestion of unprotected DNA was observed with DNase I (up to 250 units, limited by the stock concentration) in reaction buffer system. In comparison, treatment with 4,000 units of MNase eliminated the unprotected deoxynucleic acid (both dsDNA and ssDNA) and did not decrease exemplary Hem-B AAV construct recovery from intact viral particles, in all sample types tested.

qPCR Performance—The FDA guidance requires qPCR to be able to detect<50 copies of vector/1 μg genomic DNA with 95% confidence. The qPCR quantitation limit was determined by using a panel of linearized plasmid containing the exemplary Hem-B AAV construct viral genome spiked into human genomic DNA at different concentrations. Linearized plasmid at different concentrations was tested with different numbers of replicates to assess the quantification limits. N=75 was chosen for the lowest concentration to ensure a 95% detection rate with enough confidence. The lower limit of quantification (LLoQ) for qPCR was determined to be 31 copies of viral genome/1 μg genomic DNA based on the criteria of ≥95% detection, ≤20% variability and within 0.125 LOG of difference from the target value (summarized in Table 20, exceeding the FDA guidance requirement.

TABLE 20
Performance Evaluation Report Summary

| | | | | | Accuracy | Precision |
|---|---|---|---|---|---|---|
| Observed | Expected | No. of | No. | Detection | (LogObs-LogExp, | (<20% |
| (vg/1 μg gDNA) | (vg/1 μg gDNA) | Reps | Detected | Rate | Within ± 0.125LOG) | CV) |
| 455 | 500 | 22 | 22 | 100.0% | −0.041 | 10.10% |
| 42 | 50 | 12 | 12 | 100.0% | −0.075 | 14.13% |
| 31 | 37.5 | 19 | 19 | 100.0% | −0.081 | 14.74% |
| 19.4 | 25.0 | 75 | 75 | 100.0% | −0.110 | 20.47% |

The standalone sample preparation 1.0 (Siemens Healthineers SASP 1.0) system is a scalable, automated, and optimized multi-sample type preparation system that allows the barcode tracking for samples and controls. SASP 1.0 kits had increased precision with semen samples in comparison to SASP 1.2 kit, and the optimized system allows batch processing of all six biological sample types within the same run, achieving high synergy for the shedding assay.

Specificity—The specificity of the exemplary Hem-B AAV construct shedding assay was determined with 32 biological replicates each, using normal human plasma, PBMC, urine, saliva, semen and stool samples without any viral spike. In all sample types, 32/32 replicates were below quantitation limit, and the specificity was 100% in all sample types tested.

Analytical Sensitivity—During the value assignment process, linearized plasmid was used as standards in parallel with viral material diluted in a virus storage buffer processed through the extraction and qPCR. The viral stock titer was determined to be 1.53E+13 vg/mL based on the linearized plasmid standards. Performance evaluation was done in two steps. In Step 1, for each sample type, a 12-replicate panel was tested to estimate the Lower Limit of Quantitation (LLoQ); in Step 2, up to 36 biological replicates near the estimated LLoQ level were utilized to further refine LLoQ. The lowest sample concentration that demonstrated to have≤30% CV and within ±0.5 LOG of bias was determined as the LLoQ for that sample type. Exemplary Hem-B AAV construct shedding assay analytical sensitivity for each sample type is summarized in Tables 21-26 below.

TABLE 21
Analytical Sensitivity in Plasma

| Concen- | No. | | | | | LLoQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 1.0E+09 | 12 | 12 | 100.0% | 0.013 | 14.4% | 1440 |
| 1.1E+07 | 12 | 12 | 100.0% | 0.063 | 12.9% | [662-2218] |
| 1.2E+05 | 12 | 12 | 100.0% | 0.086 | 9.9% | |
| 1.4E+03 | 36 | 36 | 100.0% | 0.167 | 27.6% | |
| (LLoQ) | | | | | | |
| 1.1E+03 | 36 | 36 | 100.0% | 0.146 | 35.1% | |
| 1.1E+03 | 12 | 12 | 100.0% | 0.032 | 26.9% | |
| 6.9E+02 | 36 | 36 | 100.0% | 0.102 | 44.4% | |
| 5.3E+02 | 36 | 36 | 100.0% | 0.087 | 39.0% | |
| 5.1E+02 | 12 | 12 | 100.0% | −0.080 | 31.7% | |
| 3.4E+02 | 12 | 11 | 91.7% | 0.080 | 21.7% | |
| 8.4E+01 | 12 | 5 | 41.7% | 0.407 | 42.9% | |
| 6.8E+01 | 12 | 2 | 16.7% | 0.614 | 61.0% | |

TABLE 22
Analytical Sensitivity in PBMC*

| Concen- | | | | | | LLoQ |
|---|---|---|---|---|---|---|
| tration | No. | | | | | [95% CI] |
| (vg/1 μg | of | No. | Detection | (LogObs- | Total | (vg/1 μg |
| gDNA) | Reps | Detected | Rate | LogExp) | % CV | gDNA) |
| 4.7E+07 | 12 | 12 | 100.0% | 0.169 | 10.2% | 316 |
| 5.0E+05 | 12 | 12 | 100.0% | 0.197 | 11.4% | [176-455] |
| 5.5E+03 | 12 | 12 | 100.0% | 0.256 | 16.8% | |
| 5.3E+02 | 36 | 36 | 100.0% | 0.241 | 21.9% | |
| 4.5E+02 | 12 | 12 | 100.0% | 0.168 | 15.3% | |
| 3.2E+02 | 36 | 36 | 100.0% | 0.231 | 22.5% | |
| (LLoQ) | | | | | | |
| 1.4E+02 | 36 | 36 | 100.0% | 0.188 | 32.5% | |
| 9.4E+01 | 36 | 36 | 100.0% | 0.175 | 32.4% | |
| 7.3E+01 | 36 | 36 | 100.0% | 0.187 | 39.4% | |
| 6.2E+01 | 12 | 12 | 100.0% | 0.129 | 36.1% | |
| 3.4E+01 | 12 | 12 | 100.0% | 0.139 | 45.1% | |
| 1.3E+01 | 12 | 9 | 75.0% | 0.255 | 85.1% | |
| 9.8E+00 | 12 | 10 | 83.3% | 0.438 | 62.8% | |

*PBMCs were re-suspended in cell freezing media at the concentration of 5E+6 cells/mL.

TABLE 23
Analytical Sensitivity in Saliva

| Concen- | No. | | | | | LLoQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 1.3E+09 | 12 | 12 | 100.0% | 0.092 | 6.4% | 6172 |
| 1.4E+07 | 12 | 12 | 100.0% | 0.144 | 4.3% | [3045-9299] |
| 1.5E+05 | 12 | 12 | 100.0% | 0.171 | 9.2% | |
| 2.0E+04 | 36 | 36 | 100.0% | 0.128 | 17.0% | |
| 2.0E+04 | 12 | 12 | 100.0% | 0.124 | 15.9% | |
| 1.3E+04 | 36 | 36 | 100.0% | 0.116 | 22.5% | |
| 1.1E+04 | 12 | 12 | 100.0% | 0.145 | 18.8% | |
| 9.6E+03 | 36 | 36 | 100.0% | 0.133 | 19.6% | |
| 6.2E+03 | 36 | 36 | 100.0% | 0.087 | 25.9% | |
| (LLoQ) | | | | | | |
| 2.0E+03 | 36 | 36 | 100.0% | 0.060 | 37.5% | |
| 1.2E+03 | 12 | 12 | 100.0% | 0.076 | 37.0% | |
| 4.2E+02 | 12 | 3 | 25.0% | 0.466 | 69.1% | |
| 2.6E+02 | 12 | 5 | 41.7% | 0.572 | 13.1% | |

TABLE 24
Analytical Sensitivity in Semen

| Concen- | No. | | | | | LLoQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 1.5E+09 | 12 | 12 | 100.0% | 0.223 | 8.9% | 6037 |
| 1.4E+07 | 12 | 12 | 100.0% | 0.225 | 14.2% | [3599- |
| 1.4E+05 | 12 | 12 | 100.0% | 0.255 | 22.8% | 8475] |
| 2.3E+04 | 12 | 12 | 100.0% | 0.259 | 28.2% | |
| 2.3E+04 | 36 | 36 | 100.0% | 0.388 | 27.3% | |
| 2.0E+04 | 36 | 36 | 100.0% | 0.448 | 24.9% | |
| 1.8E+04 | 36 | 36 | 100.0% | 0.450 | 26.9% | |
| 1.6E+04 | 33 | 33 | 100.0% | 0.491 | 24.8% | |
| 6.0E+03 | 12 | 12 | 100.0% | 0.295 | 20.6% | |
| (LLoQ) | | | | | | |
| 4.2E+03 | 12 | 12 | 100.0% | 0.442 | 34.8% | |
| 1.8E+03 | 12 | 12 | 100.0% | 0.522 | 95.7% | |
| 1.3E+03 | 12 | 6 | 50.0% | 0.705 | 50.4% | |

TABLE 25
Analytical Sensitivity in Stool**

| Concen- | | | | | | LLoQ |
|---|---|---|---|---|---|---|
| tration | No. | | | | | [95% CI] |
| (vg/mg | of | No. | Detection | (LogObs- | Total | (vg/mg |
| stool) | Reps | Detected | Rate | LogExp) | % CV | stool) |
| 1.4E+07 | 12 | 12 | 100.0% | 0.115 | 14.2% | 25 |
| 1.5E+05 | 12 | 12 | 100.0% | 0.140 | 11.6% | [13-37] |
| 1.4E+03 | 12 | 12 | 100.0% | 0.144 | 13.1% | |
| 1.4E+02 | 11† | 11 | 100.0% | 0.138 | 12.6% | |
| 3.9E+01 | 36 | 36 | 100.0% | 0.109 | 28.9% | |
| 2.5E+01 | 12 | 12 | 100.0% | 0.104 | 24.8% | |
| (LLoQ) | | | | | | |
| 2.4E+01 | 36 | 36 | 100.0% | 0.070 | 38.1% | |
| 2.0E+01 | 36 | 36 | 100.0% | 0.109 | 40.8% | |
| 1.4E+01 | 12 | 12 | 100.0% | 0.131 | 31.9% | |
| 1.3E+01 | 36 | 36 | 100.0% | 0.104 | 42.8% | |
| 6.6E+00 | 12 | 11 | 91.7% | 0.168 | 49.0% | |
| 3.3E+00 | 12 | 9 | 75.0% | 0.173 | 53.5% | |

**Stool was suspended in 10 volume (w:v) of 1X PBS before extraction.
†1 sample lost due to clogging

TABLE 26
Analytical Sensitivity in Urine

| Concen- | No. | | | | | LLoQ |
|---|---|---|---|---|---|---|
| tration | of | No. | Detection | (LogObs- | Total | [95% CI] |
| (vg/mL) | Reps | Detected | Rate | LogExp) | % CV | (vg/mL) |
| 1.4E+09 | 12 | 12 | 100.0% | 0.134 | 19.0% | 5494 |
| 1.4E+07 | 12 | 12 | 100.0% | 0.135 | 9.5% | [2465-8524] |
| 1.5E+05 | 12 | 12 | 100.0% | 0.187 | 18.7% | |
| 1.1E+04 | 36 | 36 | 100.0% | 0.151 | 17.4% | |
| 1.1E+04 | 12 | 12 | 100.0% | 0.142 | 27.0% | |
| 5.5E+03 | 36 | 36 | 100.0% | 0.132 | 28.1% | |
| (LLoQ) | | | | | | |
| 4.1E+03 | 36 | 36 | 100.0% | 0.130 | 30.1% | |
| 2.8E+03 | 36 | 36 | 100.0% | 0.147 | 30.7% | |
| 2.3E+03 | 12 | 12 | 100.0% | 0.062 | 40.3% | |
| 1.3E+03 | 12 | 11 | 91.7% | 0.100 | 31.2% | |
| 6.2E+02 | 12 | 9 | 75.0% | 0.245 | 39.6% | |
| 5.9E+02 | 12 | 5 | 41.7% | 0.520 | 51.7% | |

Accuracy—Titer data were considered to be log-normally distributed and were analyzed following log 10 transformation. Exemplary Hem-B AAV construct shedding assay accuracy was determined using the same sample panels described above for analytical sensitivity. Accuracy was calculated by the difference between observed value and the expected value after log transformation, and the target requirement was with ±0.5 log bias of expected value across the quantitative range. Exemplary Hem-B AAV construct shedding assay linearity and accuracy for each sample type was summarized and presented in Tables 27-32 and FIG. 9.

TABLE 27
Accuracy in Plasma

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 1.0E+09 | 9.00 | 8.99 | 12 | 0.01 |
| 1.1E+07 | 7.06 | 7.00 | 12 | 0.06 |
| 1.2E+05 | 5.08 | 5.00 | 12 | 0.09 |
| 1.4E+03(LLoQ) | 3.16 | 2.99 | 36 | 0.17 |
| 1.1E+03 | 3.03 | 2.88 | 36 | 0.15 |
| 1.1E+03 | 3.02 | 2.99 | 12 | 0.03 |
| 6.9E+02 | 2.84 | 2.73 | 36 | 0.10 |
| 5.3E+02 | 2.72 | 2.64 | 36 | 0.09 |
| 5.1E+02 | 2.71 | 2.79 | 12 | −0.08 |
| 3.4E+02 | 2.54 | 2.46 | 12 | 0.08 |
| 8.4E+01 | 1.92 | 1.52 | 12 | 0.41 |
| 6.8E+01 | 1.83 | 1.22 | 12 | 0.61 |

TABLE 28
Accuracy in PBMC

| | Observed | Expected | | |
|---|---|---|---|---|
| | LogQTY | LogQTY | | |
| Concentration | (Log vg/μg | (Log vg/μg | No. of | Log Difference |
| (vg/1 μg gDNA) | gDNA) | gDNA) | Reps | (LogObs-LogExp) |
| 4.7E+07 | 7.67 | 7.50 | 12 | 0.17 |
| 5.0E+05 | 5.69 | 5.50 | 12 | 0.20 |
| 5.5E+03 | 3.74 | 3.48 | 12 | 0.26 |
| 5.3E+02 | 2.73 | 2.49 | 36 | 0.24 |
| 4.5E+02 | 2.65 | 2.48 | 12 | 0.17 |
| 3.2E+02 (LLoQ) | 2.50 | 2.27 | 36 | 0.23 |
| 1.4E+02 | 2.15 | 1.97 | 36 | 0.19 |
| 9.4E+01 | 1.97 | 1.79 | 36 | 0.17 |
| 7.3E+01 | 1.86 | 1.67 | 36 | 0.19 |
| 6.2E+01 | 1.79 | 1.66 | 12 | 0.13 |
| 3.4E+01 | 1.53 | 1.39 | 12 | 0.14 |
| 1.3E+01 | 1.11 | 0.86 | 12 | 0.25 |
| 9.8E+00 | 0.99 | 0.55 | 12 | 0.44 |

*PBMCs were re-suspended in cell freezing media at the concentration of 5E+6 cells/mL.

TABLE 29
Accuracy in Saliva

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 1.3E+09 | 9.11 | 9.01 | 12 | 0.09 |
| 1.4E+07 | 7.15 | 7.01 | 12 | 0.14 |
| 1.5E+05 | 5.16 | 4.99 | 12 | 0.17 |
| 2.0E+04 | 4.31 | 4.18 | 36 | 0.13 |
| 2.0E+04 | 4.29 | 4.17 | 12 | 0.12 |
| 1.3E+04 | 4.12 | 4.00 | 36 | 0.12 |
| 1.1E+04 | 4.04 | 3.90 | 12 | 0.15 |
| 9.6E+03 | 3.98 | 3.85 | 36 | 0.13 |
| 6.2E+03 (LLoQ) | 3.79 | 3.70 | 36 | 0.09 |
| 2.0E+03 | 3.30 | 3.24 | 36 | 0.06 |
| 1.2E+03 | 3.08 | 3.00 | 12 | 0.08 |
| 4.2E+02 | 2.62 | 2.16 | 12 | 0.47 |
| 2.6E+02 | 2.42 | 1.84 | 12 | 0.57 |

TABLE 30
Accuracy in Semen

| | Observed | Expected | | |
|---|---|---|---|---|
| Concentration | LogQTY | LogQTY | No. of | Log Difference |
| (vg/mL) | (Log vg/mL) | (Log vg/mL) | Reps | (LogObs-LogExp) |
| 1.5E+09 | 9.17 | 8.94 | 12 | 0.22 |
| 1.4E+07 | 7.15 | 6.93 | 12 | 0.22 |
| 1.4E+05 | 5.16 | 4.91 | 12 | 0.25 |
| 2.3E+04 | 4.36 | 4.10 | 12 | 0.26 |
| 2.3E+04 | 4.35 | 3.97 | 36 | 0.39 |
| 2.0E+04 | 4.31 | 3.86 | 36 | 0.45 |
| 1.8E+04 | 4.24 | 3.79 | 36 | 0.45 |
| 1.6E+04 | 4.20 | 3.71 | 33 | 0.49 |
| 6.0E+03 (LLoQ) | 3.78 | 3.49 | 12 | 0.29 |
| 4.2E+03 | 3.62 | 3.18 | 12 | 0.44 |
| 1.8E+03 | 3.25 | 2.73 | 12 | 0.52 |
| 1.3E+03 | 3.13 | 2.42 | 12 | 0.71 |

TABLE 31
Accuracy in Stool

| Concentration | Observed LogQTY | Expected LogQTY | | Log Difference |
|---|---|---|---|---|
| (vg/mg) | (Log vg/mg) | (Log vg/mg) | No. of Reps | (LogObs-LogExp) |
| 1.4E+07 | 7.14 | 7.02 | 12 | 0.11 |
| 1.5E+05 | 5.16 | 5.02 | 12 | 0.14 |
| 1.4E+03 | 3.15 | 3.01 | 12 | 0.14 |
| 1.4E+02 | 2.14 | 2.01 | 11† | 0.14 |
| 3.9E+01 | 1.59 | 1.48 | 36 | 0.11 |
| 2.5E+01 (LLoQ) | 1.4 | 1.3 | 12 | 0.10 |
| 2.4E+01 | 1.38 | 1.31 | 36 | 0.07 |
| 2.0E+01 | 1.29 | 1.18 | 36 | 0.11 |
| 1.4E+01 | 1.14 | 1.01 | 12 | 0.13 |
| 1.3E+01 | 1.11 | 1.01 | 36 | 0.10 |
| 6.6E+00 | 0.82 | 0.65 | 12 | 0.17 |
| 3.3E+00 | 0.52 | 0.35 | 12 | 0.17 |

**Stool was suspended in 10 volume (w:v) of 1X PBS before extraction,
†1 sample lost due to clogging

TABLE 32
Accuracy in Urine

| Concentration | Observed LogQTY | Expected LogQTY | | Log Difference |
|---|---|---|---|---|
| (vg/mL) | (Log vg/mL) | (Log vg/mL) | No. of Reps | (LogObs-LogExp) |
| 1.4E+09 | 9.15 | 9.02 | 12 | 0.13 |
| 1.4E+07 | 7.14 | 7.01 | 12 | 0.13 |
| 1.5E+05 | 5.18 | 4.99 | 12 | 0.19 |
| 1.1E+04 | 4.06 | 3.91 | 36 | 0.15 |
| 1.1E+04 | 4.04 | 3.90 | 12 | 0.14 |
| 5.5E+03 (LLoQ) | 3.74 | 3.61 | 36 | 0.13 |
| 4.1E+03 | 3.61 | 3.48 | 36 | 0.13 |
| 2.8E+03 | 3.45 | 3.31 | 36 | 0.15 |
| 2.3E+03 | 3.36 | 3.30 | 12 | 0.06 |
| 1.3E+03 | 3.10 | 3.00 | 12 | 0.10 |
| 6.2E+02 | 2.79 | 2.55 | 12 | 0.24 |
| 5.9E+02 | 2.77 | 2.25 | 12 | 0.52 |

Precision—Assay within-laboratory precision was determined using the same panels described in analytical sensitivity (described above). Each panel was tested with the same reagent lot using two instruments on multiple days. Target requirement was ≤30% CV throughout the quantitative range. Exemplary Hem-B AAV construct shedding assay within-laboratory precision for each sample type was summarized and is presented in Tables 33-38.

TABLE 33
Components of Variation for Plasm Panel

| | | Within | Between | Between | |
|---|---|---|---|---|---|
| Concentration | No. of | Run | Run | Instrument | Overall |
| (vg/mL) | Reps | (% CV) | (% CV) | (% CV) | (% CV) |
| 1.0E+09 | 12 | 6.4% | 12.7% | 12.3% | 14.4% |
| 1.1E+07 | 12 | 8.1% | 5.4% | 11.9% | 12.9% |
| 1.2E+05 | 12 | 8.0% | 3.1% | 5.9% | 9.9% |
| 1.4E+03 (LLoQ) | 36 | 21.3% | 15.0% | 14.1% | 27.6% |
| 1.1E+03 | 36 | 26.4% | 15.8% | 20.8% | 35.1% |
| 1.1E+03 | 12 | 24.7% | 13.7% | 16.2% | 26.9% |
| 6.9E+02 | 36 | 43.9% | 22.1% | 15.1% | 44.4% |
| 5.3E+02 | 36 | 38.9% | 2.9% | 14.1% | 39.0% |
| 5.1E+02 | 12 | 25.1% | 2.4% | 21.3% | 31.7% |

| | | |
|---|---|---|
| 3.4E+02 | 12 | Below assay detection range |
| 8.4E+01 | 12 | |
| 6.8E+01 | 12 | |

TABLE 34
Components of Variation for PBMC* Panel

| | | Within | Between | Between | |
|---|---|---|---|---|---|
| Concentration | No. of | Run | Run | Instrument | Overall |
| (vg/μg gDNA) | Reps | (% CV) | (% CV) | (% CV) | (% CV) |
| 4.7E+07 | 12 | 5.5% | 5.9% | 10.7% | 10.2% |
| 5.0E+05 | 12 | 7.7% | 7.4% | 11.1% | 11.4% |
| 5.5E+03 | 12 | 16.6% | 6.4% | 5.9% | 16.8% |
| 5.3E+02 | 36 | 16.1% | 7.9% | 14.1% | 21.9% |
| 4.5E+02 | 12 | 14.9% | 3.2% | 7.8% | 15.3% |
| 3.2E+02 (LLoQ) | 36 | 17.2% | 6.9% | 15.8% | 22.5% |
| 1.4E+02 | 36 | 28.8% | 16.0% | 10.2% | 32.5% |
| 9.4E+01 | 36 | 32.4% | 7.6% | 0.7% | 32.4% |
| 7.3E+01 | 36 | 31.5% | 19.5% | 19.7% | 39.4% |
| 6.2E+01 | 12 | 32.0% | 22.6% | 26.2% | 36.1% |
| 3.4E+01 | 12 | 42.6% | 25.5% | 26.4% | 45.1% |

| | | |
|---|---|---|
| 1.3E+01 | 12 | Below assay detection range |
| 9.8E+00 | 12 | |

*PBMCs were re-suspended in cell freezing media at the concentration of 5E+6 cells/mL.

TABLE 35
Components of Variation for Saliva Panel

| | | Within | Between | Between | |
|---|---|---|---|---|---|
| Concentration | No. of | Run | Run | Instrument | Overall |
| (vg/mL) | Reps | (% CV) | (% CV) | (% CV) | (% CV) |
| 1.3E+09 | 12 | 3.5% | 6.3% | 2.9% | 6.4% |
| 1.4E+07 | 12 | 4.2% | 1.1% | 0.2% | 4.3% |
| 1.5E+05 | 12 | 7.8% | 7.9% | 3.5% | 9.2% |
| 2.0E+04 | 36 | 11.7% | 4.2% | 13.0% | 17.0% |
| 2.0E+04 | 12 | 13.5% | 9.0% | 5.4% | 15.9% |
| 1.3E+04 | 36 | 15.5% | 12.7% | 14.0% | 22.5% |
| 1.1E+04 | 12 | 14.4% | 9.4% | 15.4% | 18.8% |
| 9.6E+03 | 36 | 18.0% | 8.2% | 4.9% | 19.6% |
| 6.2E+03 (LLoQ) | 36 | 24.9% | 13.2% | 6.4% | 25.9% |
| 2.0E+03 | 36 | 34.3% | 16.1% | 10.0% | 37.5% |
| 1.2E+03 | 12 | 31.9% | 23.7% | 28.4% | 37.0% |

| | | |
|---|---|---|
| 4.2E+02 | 12 | Below assay detection range |
| 2.6E+02 | 12 | |

TABLE 36
Components of Variation for Semen Panel

| | | Within | Between | Between | |
|---|---|---|---|---|---|
| Concentration | No. of | Run | Run | Instrument | Overall |
| (vg/mL) | Reps | (% CV) | (% CV) | (% CV) | (% CV) |
| 1.5E+09 | 12 | 6.8% | 6.4% | 8.3% | 8.9% |
| 1.4E+07 | 12 | 11.1% | 7.7% | 11.7% | 14.2% |
| 1.4E+05 | 12 | 9.4% | 2.5% | 22.8% | 22.8% |
| 2.3E+04 | 12 | 22.5% | 10.0% | 20.4% | 28.2% |
| 2.3E+04 | 36 | 22.6% | 8.8% | 17.1% | 27.3% |
| 2.0E+04 | 36 | 22.4% | 12.1% | 6.7% | 24.9% |
| 1.8E+04 | 36 | 23.8% | 16.0% | 4.9% | 26.9% |
| 1.6E+04 | 33 | 24.0% | 8.1% | 2.3% | 24.8% |
| 6.0E+03 (LLoQ) | 12 | 20.4% | 7.7% | 9.3% | 20.6% |
| 4.2E+03 | 12 | 32.4% | 16.5% | 19.4% | 34.8% |
| 1.8E+03 | 12 | 85.8% | 57.1% | 66.0% | 95.7% |

| | | |
|---|---|---|
| 1.3E+03 | 12 | Below assay detection range |

TABLE 37
Components of Variation for Stool** Panel

| | | Within | Between | Between | |
|---|---|---|---|---|---|
| Concentration | No. of | Run | Run | Instrument | Overall |
| (vg/mg stool) | Reps | (% CV) | (% CV) | (% CV) | (% CV) |
| 1.4E+07 | 12 | 4.8% | 7.4% | 13.4% | 14.2% |
| 1.5E+05 | 12 | 8.3% | 7.9% | 6.1% | 11.6% |
| 1.4E+03 | 12 | 8.1% | 5.7% | 10.4% | 13.1% |
| 1.4E+02 | 11† | 10.8% | 4.5% | 6.3% | 12.6% |
| 3.9E+01 | 36 | 27.5% | 11.8% | 2.7% | 28.9% |
| 2.5E+01 (LLoQ) | 12 | 24.2% | 4.8% | 13.5% | 24.8% |
| 2.4E+01 | 36 | 34.2% | 4.8% | 17.6% | 38.1% |
| 2.0E+01 | 36 | 30.8% | 10.9% | 26.4% | 40.8% |
| 1.4E+01 | 12 | 31.7% | 13.8% | 14.2% | 31.9% |
| 1.3E+01 | 36 | 40.1% | 11.8% | 17.6% | 42.8% |

| | | |
|---|---|---|
| 6.6E+00 | 12 | Below assay detection range |
| 3.3E+00 | 12 | |

**Stool was suspended in 10 volume (w:v) of 1X PBS before extraction.
†1 sample lost due to clogging

TABLE 38
Components of Variation for Urine Panel

| | | Within | Between | Between | |
|---|---|---|---|---|---|
| Concentration | No. of | Run | Run | Instrument | Overall |
| (vg/mL) | Reps | (% CV) | (% CV) | (% CV) | (% CV) |
| 1.4E+09 | 12 | 15.6% | 11.9% | 7.1% | 19.0% |
| 1.4E+07 | 12 | 7.7% | 5.9% | 3.8% | 9.5% |
| 1.5E+05 | 12 | 10.6% | 7.7% | 18.2% | 18.7% |
| 1.1E+04 | 36 | 14.9% | 10.7% | 4.7% | 17.4% |
| 1.1E+04 | 12 | 24.5% | 16.7% | 5.0% | 27.0% |
| 5.5E+03 (LLoQ) | 36 | 25.3% | 5.4% | 13.3% | 28.1% |
| 4.1E+03 | 36 | 28.5% | 21.6% | 12.5% | 30.1% |
| 2.8E+03 | 36 | 30.2% | 7.9% | 8.3% | 30.7% |
| 2.3E+03 | 12 | 37.8% | 16.1% | 20.4% | 40.3% |
| 1.3E+03 | 12 | 23.4% | 21.1% | 26.5% | 31.2% |

| | | |
|---|---|---|
| 6.2E+02 | 12 | Below assay detection range |
| 5.9E+02 | 12 | |

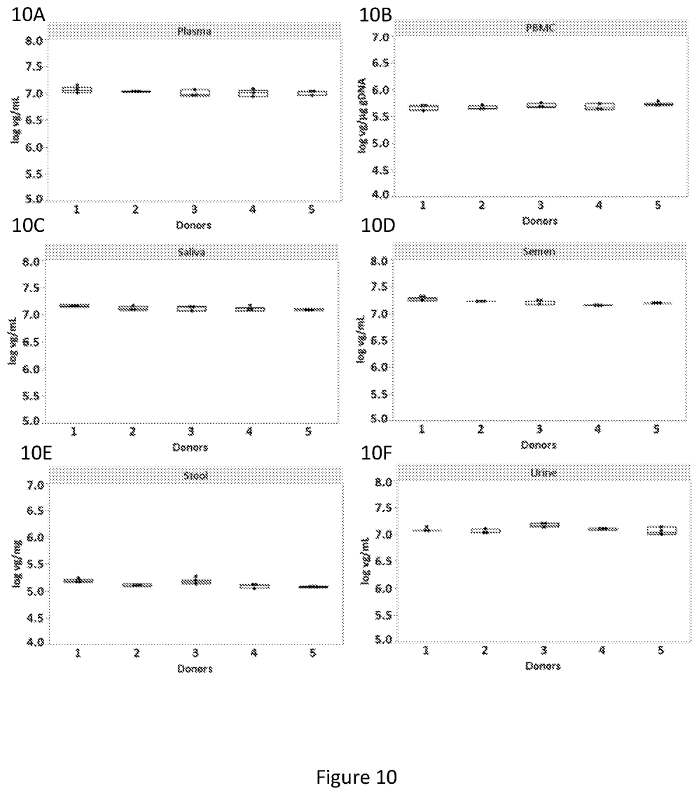

Individual Variability—Individual variability was assessed with five different donors for each sample type spiked with 1.0E+07 vg/mL exemplary Hem-B AAV construct (post 5E+6 cells/mL suspension of PBMCs and 1:10(w:v) suspension of stool). Three biological replicates for each condition were performed for the assessment. Individual variability was observed to be in the same range of assay reproducibility/precision (% CV≤30% and within ±0.5 LOG difference), data presented in Table 39, and FIG. 10.

TABLE 39
Individual Variability

| Sample Type | Donors* | Mean Titer | Log Mean Titer | % CV |
|---|---|---|---|---|
| Plasma | 1 | 1.17E+07 | 7.07 | 12.5% |
| (vg/mL) | 2 | 1.08E+07 | 7.03 | 2.6% |
| | 3 | 1.01E+07 | 7.00 | 13.9% |
| | 4 | 1.02E+07 | 7.01 | 12.8% |
| | 5 | 1.03E+07 | 7.01 | 9.9% |
| PBMC | 1 | 4.71E+05 | 5.67 | 11.4% |
| (vg/μg gDNA) | 2 | 4.62E+05 | 5.66 | 8.1% |
| | 3 | 5.02E+05 | 5.70 | 7.7% |
| | 4 | 4.74E+05 | 5.68 | 13.5% |
| | 5 | 5.31E+05 | 5.73 | 3.5% |
| Saliva | 1 | 1.45E+07 | 7.16 | 4.5% |
| (vg/mL) | 2 | 1.31E+07 | 7.12 | 8.5% |
| | 3 | 1.32E+07 | 7.12 | 9.5% |
| | 4 | 1.29E+07 | 7.11 | 6.7% |
| | 5 | 1.23E+07 | 7.09 | 2.5% |
| Semen | 1 | 1.86E+07 | 7.27 | 5.9% |
| (vg/mL) | 2 | 1.69E+07 | 7.23 | 1.0% |
| | 3 | 1.63E+07 | 7.21 | 7.2% |
| | 4 | 1.42E+07 | 7.15 | 2.1% |
| | 5 | 1.56E+07 | 7.19 | 0.5% |
| Stool | 1 | 1.54E+05 | 5.19 | 5.2% |
| (vg/mg) | 2 | 1.28E+05 | 5.11 | 5.2% |
| | 3 | 1.49E+05 | 5.17 | 8.7% |
| | 4 | 1.23E+05 | 5.09 | 8.2% |
| | 5 | 1.20E+05 | 5.08 | 3.0% |
| Urine | 1 | 1.19E+07 | 7.07 | 0.7% |
| (vg/mL) | 2 | 1.14E+07 | 7.06 | 8.5% |
| | 3 | 1.49E+07 | 7.17 | 8.5% |
| | 4 | 1.29E+07 | 7.11 | 4.9% |
| | 5 | 1.16E+07 | 7.07 | 16.6% |

*Donors for different sample types did not match; each sample types had different donors

DNase Treatment—MNase was the chosen nuclease for DNase treatment. To assess the efficiency of nuclease activity, saliva, semen, urine and 1X PBS suspended stool was spiked with three types of deoxynucleic acid templates: unprotected double-stranded DNA (dsDNA-AAVR), single-stranded DNA (ssDNA-GFP), and encapsulated viral DNA (rAAV—exemplary Hem-B AAV construct). dsDNA-AAVR and ssDNA-GFP were spiked in parallel with rAAV-exemplary Hem-B AAV construct, all at the concentration of 1.0E+07 copies/mL, to assess the efficiency of nuclease activity. MNase treatment was done in 3 replicates with 4,000 unit of MNase per sample at 37° C. for 30 min, and stopped by the addition of EDTA. Siemens storage buffer (SSB2) was spiked in the same manner, without MNase treatment, to serve as baseline for recovery calculation. After the MNase reaction was complete, DNA extraction was performed in accordance with methods known in the art.

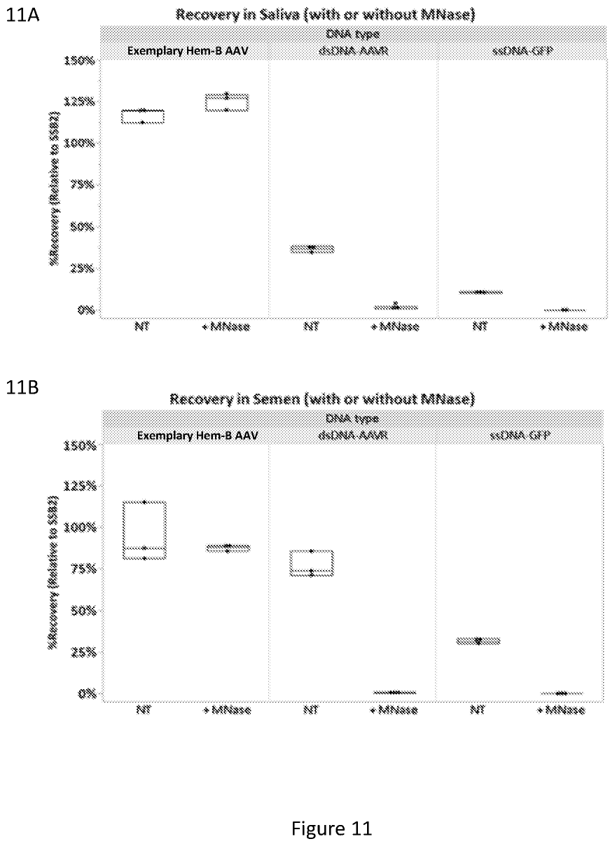

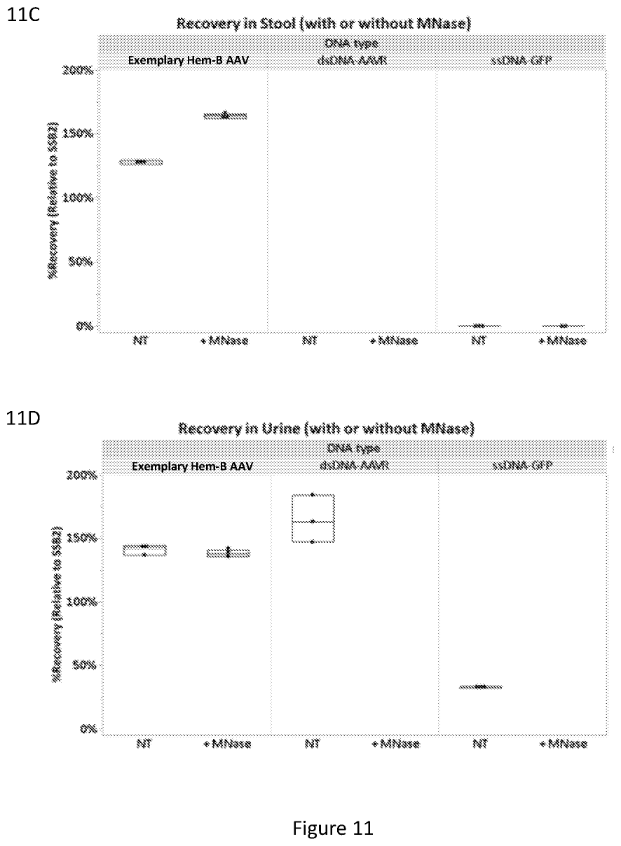

Strong endogenous nuclease activity toward unprotected deoxynucleic acids, both dsDNA and ssDNA, was observed in PBS suspended stool samples. Moderate endogenous nuclease activity toward ssDNA was also observed in saliva, and to a lesser extent in semen and urine samples. MNase treatment in all sample types depleted unprotected dsDNA and ssDNA templates (>97.5% reduction) without decreasing rAAV recovery. Exemplary Hem-B AAV construct MNase treatment results are summarized in FIG. 11 and Table 40.

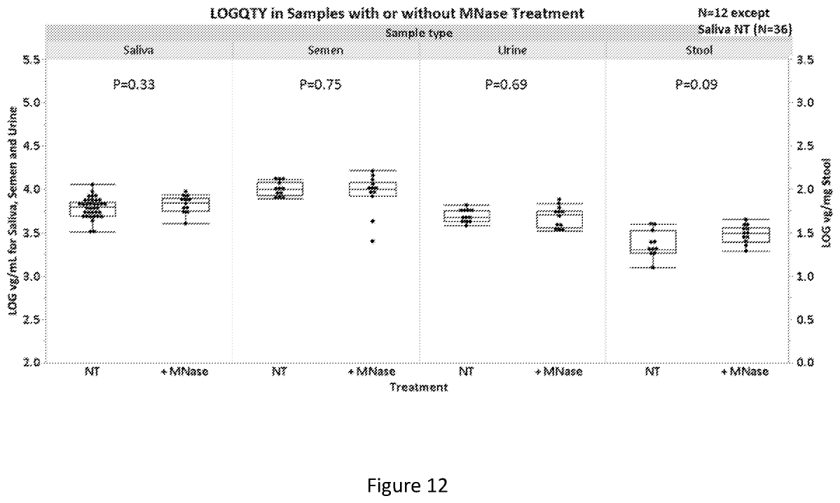

The effect of MNase treatment on intact viral DNA recovery was further assessed in all sample types at a concentration near the LLoQ, with a minimum of 12 replicates. No significant difference was observed between MNase treated or untreated in all sample types, results are summarized in FIG. 12, and Table 41.

TABLE 40
Summary of rAAV, dsDNA and ssDNA Percent Recovery with or without
MNase Treatment in Tested Sample Types

| | Percent Recovery (Relative to Spiked Untreated Siemens Storage Buffer) | | | | | |
|---|---|---|---|---|---|---|
| | rAAV (HemB AAV, | dsDNA (AAVR, | | | | |
| Sample | protected) | unprotected) | ssDNA (GFP, unprotected) | | | |
| type | Not Treated | +MNase | Not Treated | +MNase | Not Treated | +MNase |
| Saliva | 117 ± 4% | 125 ± 5% | 37 ± 2% | 2 ± 1% | 11 ± 1% | < 1% |
| Semen | 95 ± 18% | 88 ± 2% | 77 ± 8% | <1% | 32 ± 1% | < 1% |
| Stool | 128 ± 2% | 164 ± 2% | ND | ND | <1% | < 1% |
| Urine | 141 ± 4% | 138 ± 2% | 165 ± 19% | ND | 33 ± 1% | ND |

ND = not detected by qPCR

TABLE 41
Summary of Exemplary HemB AAV Construct Recovery with or without
MNase Treatment in Each Sample Type near LLoQ

| | HemB AAV | | |
|---|---|---|---|
| Sample type | Not Treated | +MNase | P value |
| Saliva (LOG vg/mL) | 3.78 ± 0.11 | 3.82 ± 0.10 | 0.33 |
| Semen (LOG vg/mL) | 4.00 ± 0.08 | 3.95 ± 0.23 | 0.75 |
| Urine (LOG vg/mL) | 3.69 ± 0.08 | 3.67 ± 0.11 | 0.69 |
| Stool (LOG vg/mg) | 1.37 ± 0.16 | 1.48 ± 0.11 | 0.09 |

Analyte Stability—Stability of exemplary Hem-B AAV construct was analyzed in both unprocessed whole blood and processed specimens—plasma, PBMCs, urine, saliva, semen and stool, under different conditions.

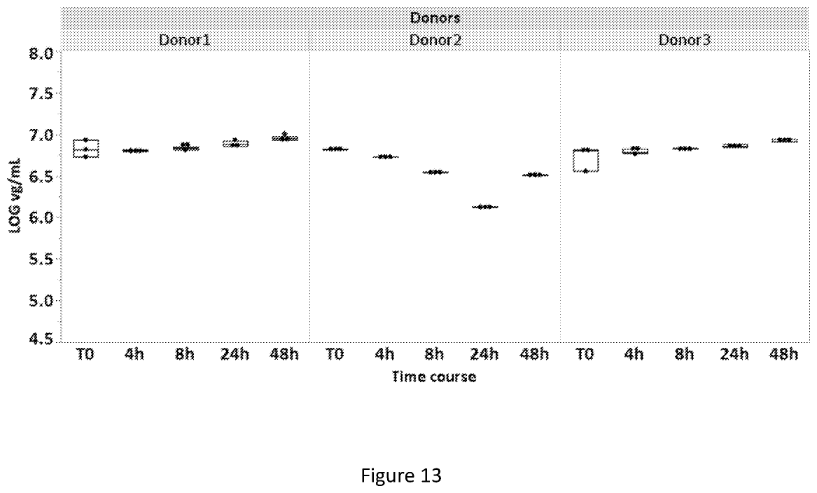

Room Temperature Stability of Unprocessed Specimen—Whole Blood—Stability of exemplary Hem-B AAV construct in whole blood stored at RT was determined using blood freshly drawn from 3 individual donors and spiked with 1.0E+07 vg/mL of exemplary Hem-B AAV construct. Plasma was isolated after storing whole blood at RT for indicated time and kept in −80° C. until the last time point was collected for plasma shedding assay analysis. Three biological replicates were tested for each condition. Individual to individual variability was observed; one of the donors showed a 0.3-0.7 LOG decrease after the 24 hr RT incubation. Stability results are summarized in FIG. 13, and Table 42.

TABLE 42
Stability of Exemplary HemB AAV
Construct in Whole Blood Stored at RT

| | | | Log Mean | |
|---|---|---|---|---|
| Donors | Time(h) | Mean Titer | Titer | % CV |
| Donor1 | 0 | 6.85E+06 | 6.83 | 23.6% |
| (vg/mL) | 4 | 6.38E+06 | 6.80 | 2.8% |
| | 8 | 6.85E+06 | 6.84 | 4.5% |
| | 24 | 7.74E+06 | 6.89 | 7.4% |
| | 48 | 8.98E+06 | 6.95 | 4.7% |
| | 0 | 6.85E+06 | 6.83 | 1.3% |
| Donor2 | 4 | 6.38E+06 | 6.73 | 1.3% |
| (vg/mL) | 8 | 6.85E+06 | 6.55 | 1.3% |
| | 24 | 7.74E+06 | 6.13 | 0.9% |
| | 48 | 8.98E+06 | 6.51 | 0.8% |
| Donor3 | 0 | 5.54E+06 | 6.74 | 30.1% |
| (vg/mL) | 4 | 6.18E+06 | 6.79 | 6.5% |
| | 8 | 6.73E+06 | 6.83 | 1.5% |
| | 24 | 7.30E+06 | 6.86 | 3.3% |
| | 48 | 8.61E+06 | 6.93 | 4.0% |

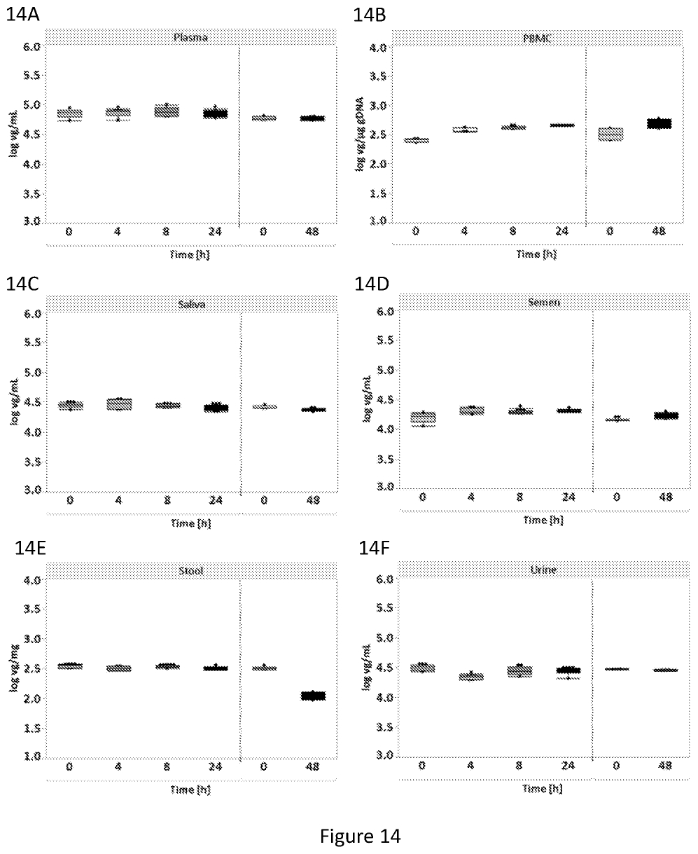

Room Temperature Stability of Unprocessed Specimen—Plasma, PBMCs, Saliva, Semen, Urine and Stool—Stability of exemplary Hem-B AAV construct virus plasma, PBMCs, saliva, semen, stool and urine, stored at RT was assessed at different time points. Samples pooled from five donors were spiked with 1.0E+05 vg/mL exemplary Hem-B AAV construct (post 5E+6 cells/mL suspension of PBMCs and 1:10 (w:v) suspension of stool), and three biological replicates per condition were tested at indicated time points. Exemplary Hem-B AAV construct viral genome titer was within ±0.5 LOG of the titer determined at time 0 for up to 48 hours except stool, which was stable up to 24 hours. See results summary in FIG. 14, and Table 43.

TABLE 43
Stability Samples Stored at RT

| Sample Type | Time (h) | Mean Titer | Log Mean Titer | % CV |
|---|---|---|---|---|
| Plasma | 0 | 7.19E+04 | 4.86 | 15.0% |
| (vg/mL) | 4 | 7.42E+04 | 4.87 | 14.8% |
| | 8 | 7.80E+04 | 4.89 | 16.9% |
| | 24 | 7.20E+04 | 4.86 | 14.3% |
| | 0 | 5.90E+04 | 4.77 | 7.2% |
| | 48 | 6.00E+04 | 4.78 | 7.9% |
| PBMC | 0 | 2.55E+02 | 2.41 | 7.7% |
| (vg/μg gDNA) | 4 | 3.80E+02 | 2.58 | 9.4% |
| | 8 | 4.23E+02 | 2.63 | 4.7% |
| | 24 | 4.60E+02 | 2.66 | 3.8% |
| | 0 | 3.28E+02 | 2.52 | 22.9% |
| | 48 | 4.81E+02 | 2.68 | 20.0% |
| Saliva | 0 | 2.81E+04 | 4.45 | 9.6% |
| (vg/mL) | 4 | 3.02E+04 | 4.48 | 17.0% |
| | 8 | 2.77E+04 | 4.44 | 7.2% |
| | 24 | 2.59E+04 | 4.41 | 11.4% |
| | 0 | 2.61E+04 | 4.42 | 5.6% |
| | 48 | 2.41E+04 | 4.38 | 6.8% |
| Semen | 0 | 1.59E+04 | 4.20 | 17.3% |
| (vg/mL) | 4 | 2.03E+04 | 4.31 | 10.9% |
| | 8 | 1.98E+04 | 4.30 | 7.3% |
| | 24 | 2.01E+04 | 4.30 | 4.6% |
| | 0 | 1.44E+04 | 4.16 | 2.6% |
| | 48 | 1.68E+04 | 4.23 | 12.5% |
| Stool | 0 | 3.53E+02 | 2.55 | 6.4% |
| (vg/mg) | 4 | 3.18E+02 | 2.50 | 7.7% |
| | 8 | 3.45E+02 | 2.54 | 4.4% |
| | 24 | 3.20E+02 | 2.51 | 5.0% |
| | 0 | 3.19E+02 | 2.50 | 5.5% |
| | 48 | 1.12E+02 | 2.05 | 15.0% |
| Urine | 0 | 3.11E+04 | 4.49 | 9.9% |
| (vg/mL) | 4 | 2.21E+04 | 4.34 | 8.7% |
| | 8 | 2.79E+04 | 4.45 | 14.5% |
| | 24 | 2.83E+04 | 4.45 | 14.2% |
| | 0 | 2.98E+04 | 4.47 | 2.7% |
| | 48 | 2.86E+04 | 4.46 | 2.9% |

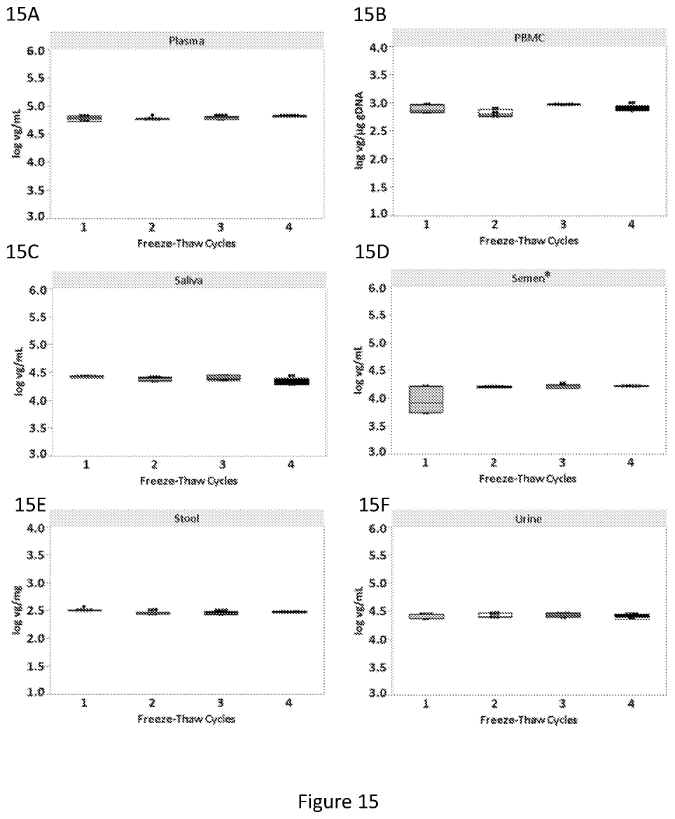

Freeze/Thaw Stability of Plasma, PBMCs, Saliva, Semen, Urine and Stool—Stability of exemplary Hem-B AAV construct virus in plasma, PBMCs, urine, saliva, semen and stool for up to 4 freeze/thaw cycles was assessed. Samples pooled from five donors were spiked at 1.0E+05 vg/mL (post 5E+6 cells/mL suspension of PBMCs and 1:10 (w:v) suspension of stool), and three biological replicates per condition were tested. Exemplary Hem-B AAV construct viral genome titer was within ±0.5 LOG of starting condition up to 4 freeze-thaw cycles. See results summary in FIG. 15, and Table 44.

TABLE 44
Summary of Sample Freeze/Thaw Stability

| | Freeze Thaw | | | |
|---|---|---|---|---|
| Sample Type | Cycles | Mean Titer | Log Mean Titer | % CV |
| Plasma | 1 | 6.02E+04 | 4.78 | 8.0% |
| (vg/mL) | 2 | 5.87E+04 | 4.77 | 2.4% |
| | 3 | 6.16E+04 | 4.79 | 5.2% |
| | 4 | 6.65E+04 | 4.82 | 3.2% |
| PBMC | 1 | 7.76E+02 | 2.89 | 15.5% |
| (vg/μg gDNA) | 2 | 6.58E+02 | 2.82 | 12.3% |
| | 3 | 9.29E+02 | 2.97 | 2.3% |
| | 4 | 8.15E+02 | 2.91 | 11.2% |
| Saliva | 1 | 2.68E+04 | 4.43 | 4.1% |
| (vg/mL) | 2 | 2.42E+04 | 4.38 | 7.0% |
| | 3 | 2.52E+04 | 4.40 | 9.6% |
| | 4 | 2.27E+04 | 4.36 | 11.6% |
| Semen | 1 | 1.01E+04 | 4.00 | 50.3%* |
| (vg/mL) | 2 | 1.62E+04 | 4.21 | 2.9% |
| | 3 | 1.63E+04 | 4.21 | 7.5% |
| | 4 | 1.65E+04 | 4.22 | 1.8% |
| Stool | 1 | 3.23E+02 | 2.51 | 2.3% |
| (vg/mg) | 2 | 2.84E+02 | 2.45 | 3.3% |
| | 3 | 2.88E+02 | 2.46 | 4.6% |
| | 4 | 2.99E+02 | 2.48 | 4.2% |
| Urine | 1 | 2.64E+04 | 4.42 | 9.5% |
| (vg/mL) | 2 | 2.66E+04 | 4.42 | 8.5% |
| | 3 | 2.65E+04 | 4.42 | 8.0% |
| | 4 | 2.63E+04 | 4.42 | 7.0% |

SASP1.2 kit was used for extraction and relative high variation was observed in the semen sample with 1 freeze/thaw cycle.

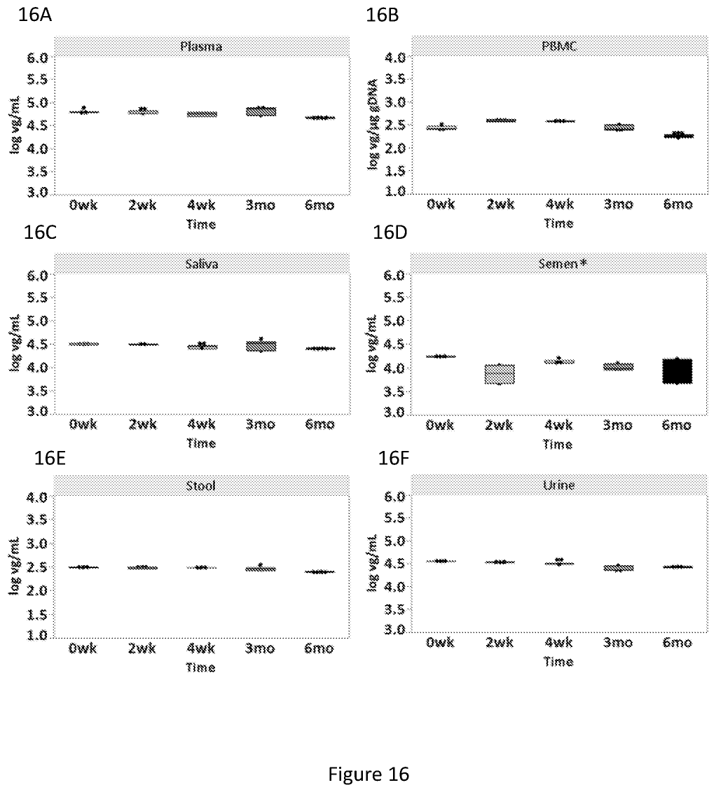

Long-term −80° C. Storage Stability of Plasma, PBMCs, Saliva, Semen, Urine and Stool—Stability of exemplary Hem-B AAV construct virus in plasma, PBMCs, saliva, semen, stool and urine stored at −80° C. was assessed for up to 6 months. Pooled samples were spiked at 1.0E+05 vg/mL (post 5E+6 cells/mL suspension of PBMCs and 1:10 (w:v) suspension of stool), and three biological replicates per condition were tested at indicated time points. Exemplary Hem-B AAV construct viral genome titer was within ±0.5 LOG of the titer determined at time 0 for up to 6 months. Long-term −80° C. Storage Stability results are summary in FIG. 16, and Table 45.

TABLE 45
Summary of Sample Stability with Long-term −80° C. Storage

| Sample Type | Time | Mean Titer | Log Mean Titer | % CV |
|---|---|---|---|---|
| Plasma | 0 wk | 6.20E+04 | 4.79 | 2.6% |
| (vg/mL) | 2 wk | 6.34E+04 | 4.80 | 7.3% |
| | 4 wk | 5.68E+04 | 4.75 | 8.5% |
| | 3 mo | 6.81E+04 | 4.83 | 19.4% |
| | 6 mo | 4.71E+04 | 4.67 | 3.7% |
| PBMC (vg/μg | 0 wk | 2.72E+02 | 2.43 | 8.4% |
| gDNA) | 2 wk | 3.98E+02 | 2.60 | 5.9% |
| | 4 wk | 3.85E+02 | 2.59 | 0.9% |
| | 3 mo | 2.73E+02 | 2.44 | 14.5% |
| | 6 mo | 1.85E+02 | 2.27 | 7.2% |
| Saliva (vg/mL) | 0 wk | 3.15E+04 | 4.50 | 2.4% |
| | 2 wk | 3.13E+04 | 4.50 | 3.8% |
| | 4 wk | 2.79E+04 | 4.45 | 6.8% |
| | 3 mo | 3.03E+04 | 4.48 | 22.7% |
| | 6 mo | 2.49E+04 | 4.40 | 4.0% |
| Semen (vg/mL) | 0 wk | 1.74E+04 | 4.24 | 3.3% |
| | 2 wk | 7.86E+03 | 3.90 | 43.2%* |
| | 4 wk | 1.35E+04 | 4.13 | 6.5% |
| | 3 mo | 1.05E+04 | 4.02 | 17.1% |
| | 6 mo | 9.78E+03 | 3.99 | 54.0%* |
| Stool (vg/mg) | 0 wk | 3.12E+02 | 2.49 | 1.8% |
| | 2 wk | 3.12E+02 | 2.49 | 3.9% |
| | 4 wk | 3.06E+02 | 2.49 | 0.5% |
| | 3 mo | 2.86E+02 | 2.46 | 8.1% |
| | 6 mo | 2.45E+02 | 2.39 | 1.6% |
| Urine (vg/mL) | 0 wk | 3.60E+04 | 4.56 | 1.1% |
| | 2 wk | 3.41E+04 | 4.53 | 0.9% |
| | 4 wk | 3.18E+04 | 4.50 | 3.1% |
| | 3 mo | 2.47E+04 | 4.39 | 12.8% |
| | 6 mo | 2.69E+04 | 4.43 | 3.0% |

*SASP1.2 kit was used for extraction and relative high variation was observed in the semen samples at the 2 wk and 6 mo time points

### REFERENCES

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.
Design and Analysis of Shedding Studies for Virus or Bacteria Based Gene Therapy and Oncolytic Products, Guidance for Industry—FDA, 2015.
Entering the Modern Era of Gene Therapy—Anguela & High, Annu Rev Med. 2019
Guidelines on Scientific Requirements for the Environmental Risk Assessment of Gene Therapy Medicinal Products—GTWP, BWP, and SWP. 2008
International Conference Harmonization considerations, General Principles to Address Virus and Vector Shedding—ICH, 2009
Malaria parasite evolution in a test tube—Carlton. Science. 2018
Pharmaceutical Development of AAV-Based Gene Therapy Products for the Eye—Rodrigues et al., Pharm Res. 2018
Recommendations for Regulating the Environmental Risk of Shedding for Gene Therapy—Bubela et al. Front Med. 2019
Mendell J R, Shilling C, Leslie N D, Flanigan K M, al-Dahhak R, Gastier-Foster J, et al. Evidence-based path to newborn screening for Duchenne muscular dystrophy. Ann Neurol. 2012; 71(3):304-13. doi: 10.1002/ana.23528.
Moat S J, Bradley D M, Salmon R, Clarke A, Hartley L. Newborn bloodspot screening for Duchenne muscular dystrophy: 21 years experience in Wales (UK) European journal of human genetics: EJHG. 2013; 21(10):1049-53. doi: 10.1038/ejhg.2012.301.
Duchenne G-B-A. Paraplegie hypertrophique de l'enfance de cause cerebrale1861.
Birnkrant D J, Bushby K, Bann C M, Apkon S D, Blackwell A, Brumbaugh D, et al. Diagnosis and management of Duchenne muscular dystrophy, part 1: diagnosis, and neuromuscular, rehabilitation, endocrine, and gastrointestinal and nutritional management. Lancet Neurol. 2018; 17(3):251-67. doi: 10.1016/S1474-4422(18)30024-3
Birnkrant D J, Bushby K, Bann C M, Alman B A, Apkon S D, Blackwell A, et al. Diagnosis and management of Duchenne muscular dystrophy, part 2: respiratory, cardiac, bone health, and orthopaedic management. Lancet Neurol. 2018; 17(4):347-61. doi: 10.1016/S1474-4422(18)30025-5.
Birnkrant D J, Bushby K, Bann C M, Apkon S D, Blackwell A, Colvin M K, et al. Diagnosis and management of Duchenne muscular dystrophy, part 3: primary care, emergency management, psychosocial care, and transitions of care across the lifespan. Lancet Neurol. 2018; 17(5):445-55. doi: 10.1016/S1474-4422(18)30026-7
Shieh P B. Emerging Strategies in the Treatment of Duchenne Muscular Dystrophy. Neurotherapeutics. 2018; 15(4):840-848. doi:10.1007/s13311-018-00687-z
Guidance for Industry: Design and Analysis of Shedding Studies for Virus or Bacteria-Based Gene Therapy and Oncolytic Products, FDA, August 2015
Guidance for Industry: Gene Therapy Clinical Trials—Observing Subjects for Delayed Adverse Events, FDA, November 2006

1. A composition comprising a collection of primers that comprises:
(a) one or more forward primers comprising a forward primer with a nucleotide sequence according to SEQ ID NO: 1 or an active fragment thereof; and
(b) one or more reverse primers comprising a reverse primer with a nucleotide sequence according to SEQ ID NO: 11 or an active fragment thereof.

2. The composition of claim 1, further comprising:
one or more probes comprising a probe with a nucleotide sequence according to SEQ ID NO: 17 or an active fragment thereof.

3. The composition of claim 1, wherein the one or more forward primers, the one or more reverse primers, the one or more probes or a combination thereof is DNA.

4. The composition of claim 1, wherein the one or more forward primers, the one or more reverse primers, the one or more probes, or a combination thereof comprises a detectable label.

5. The composition of claim 4, wherein the detectable label does not comprise nucleotides.

6. The composition of claim 4, wherein the detectable label is a fluorescent moiety.

7. The composition of claim 6, wherein the probe comprises a fluorescent moiety.

8. The composition of claim 7, wherein the probe comprises one or more quenchers.

9. The composition of claim 1, wherein an active fragment is
(a) at least 10 nucleotides in length;
(b) at least 15 nucleotides in length; or
(c) at least 20 nucleotides in length.

10. The composition of claim 9, wherein the composition further comprises a sample obtained from a subject who has been administered a Duchenne Muscular Dystrophy (DMD) AAV construct, wherein the DMD AAV construct comprises one or more regulatory elements and a therapeutic gene of interest, and wherein the sample comprises nucleic acids.

11. The composition of claim 10, wherein the nucleic acids in the sample comprise the DMD AAV construct or a fragment thereof.

12. The composition of claim 10, wherein the one or more regulatory elements comprises an enhancer, a promoter, a polyA signal sequence, or a combination thereof.

13. The composition of claim 10, wherein the therapeutic gene of interest comprises microdystrophin.

14. The composition of claim 10, further comprising a plurality of amplicons, wherein each amplicon comprises:
(a) a first strand comprising:
(i) a nucleotide sequence corresponding to the forward primer, and
(ii) a nucleotide sequence corresponding to a portion of the DMD AAV construct or fragment thereof;
(b) a second strand comprising:
(i) a nucleotide sequence of the reverse primer, and
(ii) a nucleotide sequence that is complementary to the portion of the DMD AAV construct or fragment thereof; or
(c) a combination thereof; and
wherein the portion of the DMD AAV construct or fragment thereof comprises a nucleotide sequence that spans a junction between two regulatory elements or a junction between a regulatory element and the therapeutic gene of interest.

15. The composition of claim 14, wherein the probe with a nucleotide sequence according to SEQ ID NO: 17 or an active fragment thereof is capable of hybridizing with the amplicon.

16. The composition of claim 15, wherein a level of probe-amplicon hybridization is detectable.

17. The composition of claim 15, wherein a level of probe-amplicon hybridization is quantifiable.

18. A method comprising:
(a) contacting a sample obtained from a subject with a composition according to claim 1, wherein the subject has been administered a DMD AAV construct, wherein the DMD AAV construct comprises one or more regulatory elements and a therapeutic gene of interest; and
(b) amplifying a target sequence to generate a plurality of amplicons, wherein the target sequence is a DMD AAV construct or fragment thereof and each amplicon comprises:
(i) a first strand comprising:
(1) a nucleotide sequence corresponding to the forward primer, and
(2) a nucleotide sequence corresponding to a portion of the DMD AAV construct or fragment thereof;
(ii) a second strand comprising:
(1) a nucleotide sequence of the reverse primer, and
(2) a nucleotide sequence that is complementary to the portion of the DMD AAV construct or fragment thereof; or
(iii) a combination thereof; and
wherein the portion of the DMD AAV construct or fragment thereof comprises a nucleotide sequence that spans a junction between two regulatory elements or a junction between a regulatory element and the therapeutic gene of interest.

19. The method of claim 18, wherein the composition comprises a plurality of probes each with a nucleotide sequence according to SEQ ID NO: 17 or an active fragment thereof.

20. The method of claim 19, further comprises detecting a level of hybridization between the plurality of probes and the plurality of amplicons.

21. The method of claim 20, wherein the level of hybridization between the plurality of probes and the plurality of amplicons indicates a quantity of DMD AAV construct in the sample.

22. A composition comprising a collection of primers that comprises:
(a) one or more forward primers comprising a forward primer with a nucleotide sequence according to SEQ ID NO: 35 or an active fragment thereof; and
(b) one or more reverse primers comprising a reverse primer with a nucleotide sequence according to SEQ ID NO: 45 or an active fragment thereof.

23. The composition of claim 22, further comprising:
one or more probes comprising a probe with a nucleotide sequence according to SEQ ID NO: 55 or an active fragment thereof.

24. The composition of claim 22, wherein the one or more forward primers, the one or more reverse primers, the one or more probes, or a combination thereof is DNA.

25. The composition of claim 22, wherein the one or more forward primers, the one or more reverse primers, the one or more probes, or a combination thereof comprises a detectable label.

26. The composition of claim 25, wherein the detectable label does not comprise nucleotides.

27. The composition of claim 25, wherein the detectable label is a fluorescent moiety.

28. The composition of claim 22, wherein the probe comprises a fluorescent moiety.

29. The composition of claim 28, wherein the probe comprises one or more quenchers.

30. The composition of claim 22, wherein an active fragment is
(a) at least 10 nucleotides in length;
(b) at least 15 nucleotides in length; or
(c) at least 20 nucleotides in length.

31. The composition of claim 22, wherein the composition further comprises a sample obtained from a subject has been administered a hemophilia B (Hem-B) AAV construct, wherein the Hem-B AAV construct comprises one or more regulatory elements and a therapeutic gene of interest, and wherein the sample comprises nucleic acids.

32. The composition of claim 31, wherein the nucleic acids in the sample comprise a Hem-B AAV construct or a fragment thereof.

33. The composition of claim 31, wherein the one or more regulatory elements comprises an enhancer, a promoter, a polyA signal sequence, or a combination thereof.

34. The composition of claim 31, wherein the therapeutic gene of interest comprises Factor IX.

35. The composition of claim 31, wherein the composition further comprises a plurality of amplicons, wherein each amplicon comprises:
(a) a first strand comprising:
(i) a nucleotide sequence corresponding to the forward primer, and
(ii) a nucleotide sequence corresponding to a portion of the Hem-B AAV construct or fragment thereof;
(b) a second strand comprising:
(i) a nucleotide sequence of the reverse primer, and
(ii) a nucleotide sequence that is complementary to the portion of the Hem-B AAV construct or fragment thereof; or
(c) a combination thereof; and
wherein the portion of the Hem-B AAV construct or fragment thereof comprises a nucleotide sequence that spans a junction between two regulatory elements or a junction between a regulatory element and the therapeutic gene of interest.

36. The composition of claim 35, wherein the probe with a nucleotide sequence according to SEQ ID NO: 55 or an active fragment thereof is capable of hybridizing with the amplicon.

37. The composition of claim 36, wherein a level of probe-amplicon hybridization is detectable.

38. The composition of claim 36, wherein a level of probe-amplicon hybridization is quantifiable.

39. A method comprising:
(a) contacting a sample obtained from a subject with a composition according to claim 22, wherein the subject has been administered a Hem-B AAV construct, wherein the Hem-B AAV construct comprises one or more regulatory elements and a therapeutic gene of interest; and
(b) amplifying a target sequence to generate a plurality of amplicons, wherein the target sequence is a Hem-B AAV construct or fragment thereof and each amplicon comprises:
(i) a first strand comprising:
(1) a nucleotide sequence corresponding to the forward primer, and
(2) a nucleotide sequence corresponding to a portion of a strand of the Hem-B AAV construct or fragment thereof;
(ii) a second strand comprising:
(1) a nucleotide sequence of the reverse primer, and
(2) a nucleotide sequence that is complementary to a portion of a strand of the Hem-B AAV construct or fragment thereof; or
(iii) a combination thereof; and
wherein the portion of the Hem-B AAV construct or fragment thereof comprises a nucleotide sequence that spans a junction between two regulatory elements or a junction between a regulatory element and the therapeutic gene of interest.

40. The method of claim 39, wherein the composition comprises a plurality of probes each with a nucleotide sequence according to SEQ ID NO: 55 or an active fragment thereof.

41. The method of claim 40, further comprises detecting a level of hybridization between the plurality of probes and the plurality of amplicons.

42. The method of claim 41, wherein the level of hybridization between the plurality of probes and the plurality of amplicons indicates a quantity of DMD AAV construct in the sample.

43. A method comprising:
(a) contacting a sample obtained from a subject with a composition comprising a first primer and a second primer, wherein the subject has been previously administered an AAV construct comprising one or more regulatory elements and a gene of interest, wherein the first primer comprises a sequence corresponding to or complementary to the gene of interest and the second primer comprises a sequence corresponding to or complementary to a regulatory element; and
(b) performing a polymerase chain reaction to generate a plurality of amplicons, wherein the target sequence is the AAV construct or fragment thereof and each amplicon comprises:
(i) a first strand comprising:
(1) a nucleotide sequence corresponding to the forward primer, and
(2) a nucleotide sequence corresponding to a portion of the AAV construct or fragment thereof;
(ii) a second strand comprising:
(1) a nucleotide sequence of the reverse primer, and
(2) a nucleotide sequence that is complementary to the portion of the AAV construct or fragment thereof; or
(iii) a combination thereof;
wherein the portion of the AAV construct or fragment thereof comprises a nucleotide sequence that spans a junction between a regulatory element and the therapeutic gene of interest.

44. A method comprising:
(a) contacting a sample obtained from a subject with a composition comprising a first primer and a second primer, wherein the subject has been previously administered an AAV construct comprising two or more regulatory elements and a gene of interest, wherein the first primer comprises a sequence corresponding to or complementary to a first regulatory element and the second primer comprises a sequence corresponding to or complementary to a second regulatory element; and
(b) performing a polymerase chain reaction to generate a plurality of amplicons, wherein the target sequence is the AAV construct or fragment thereof and each amplicon comprises:
(i) a first strand comprising:
(1) a nucleotide sequence corresponding to the forward primer, and
(2) a nucleotide sequence corresponding to a portion of the AAV construct or fragment thereof;
(ii) a second strand comprising:
(1) a nucleotide sequence of the reverse primer, and
(2) a nucleotide sequence that is complementary to the portion of the AAV construct or fragment thereof; or
(iii) a combination thereof;
wherein the portion of the AAV construct or fragment thereof comprises a nucleotide sequence that spans a junction between the first regulatory element and the second regulatory element.


SEQUENCE LISTING (92 sequences)

SEQ ID NO: 1 - DNA, 23 bp, Artificial Sequence
  Description of Artificial Sequence Synthetic oligonucleotide
agacagacactcaggagccagcc

SEQ ID NO: 2 - RNA, 23 nt, Artificial Sequence
  Description of Artificial Sequence Synthetic oligonucleotide
agacagacacucaggagccagcc

SEQ ID NO: 3 - DNA, 23 bp, Artificial Sequence
  Description of Artificial Sequence Synthetic oligonucleotide
accacctccacagcacagacaga

SEQ ID NO: 4 - RNA, 23 nt, Artificial Sequence
  Description of Artificial Sequence Synthetic oligonucleotide
accaccuccacagcacagacaga

SEQ ID NO: 5 - DNA, 23 bp, Artificial Sequence
  Description of Artificial Sequence Synthetic oligonucleotide
cctactacatcaaccacgagacc

SEQ ID NO: 6 - RNA, 23 nt, Artificial Sequence
  Description of Artificial Sequence Synthetic oligonucleotide
ccuacuacaucaaccacgagacc

SEQ ID NO: 7 - DNA, 23 bp, Artificial Sequence
  Description of Artificial Sequence Synthetic oligonucleotide
ggataagtaccgctacctgttca

SEQ ID NO: 8 - RNA, 23 nt, Artificial Sequence
  Description of Artificial Sequence Synthetic oligonucleotide
ggauaaguaccgcuaccuguuca

SEQ ID NO: 9 - DNA, 23 bp, Artificial Sequence
  Description of Artificial Sequence Synthetic oligonucleotide
agcagtcctccacttcctcccac

SEQ ID NO: 10 - RNA, 23 nt, Artificial Sequence
  Description of Artificial Sequence Synthetic oligonucleotide
agcaguccuccacuuccucccac

SEQ ID NO: 11 - DNA, 23 bp, Artificial Sequence
  Description of Artificial Sequence Synthetic oligonucleotide
tgcacgtcctctctctcgtagca

SEQ ID NO: 12 - RNA, 23 nt, Artificial Sequence
  Description of Artificial Sequence Synthetic oligonucleotide
ugcacguccucucucucguagca

SEQ ID NO: 13 - DNA, 23 bp, Artificial Sequence
  Description of Artificial Sequence Synthetic oligonucleotide
ctagggatctggatgctatcgtg

SEQ ID NO: 14 - RNA, 23 nt, Artificial Sequence
  Description of Artificial Sequence Synthetic oligonucleotide
cuagggaucuggaugcuaucgug

SEQ ID NO: 15 - DNA, 23 bp, Artificial Sequence
  Description of Artificial Sequence Synthetic oligonucleotide
ctctgatacagctcggtcatctt

SEQ ID NO: 16 - RNA, 23 nt, Artificial Sequence
  Description of Artificial Sequence Synthetic oligonucleotide
cucugauacagcucggucaucuu

SEQ ID NO: 17 - DNA, 16 bp, Artificial Sequence
  Description of Artificial Sequence Synthetic oligonucleotide
cggccgatccgccacc

SEQ ID NO: 18 - RNA, 16 nt, Artificial Sequence
  Description of Artificial Sequence Synthetic oligonucleotide
cggccgauccgccacc

SEQ ID NO: 19 - DNA, 25 bp, Artificial Sequence
  Description of Artificial Sequence Synthetic oligonucleotide
agcgtcgagcggccgatccgccacc

SEQ ID NO: 20 - RNA, 25 nt, Artificial Sequence
  Description of Artificial Sequence Synthetic oligonucleotide
agcgucgagcggccgauccgccacc

SEQ ID NO: 21 - DNA, 14 bp, Artificial Sequence
  Description of Artificial Sequence Synthetic oligonucleotide
atggtggcggatcg

SEQ ID NO: 22 - RNA, 14 nt, Artificial Sequence
  Description of Artificial Sequence Synthetic oligonucleotide
augguggcggaucg

SEQ ID NO: 23 - DNA, 23 bp, Artificial Sequence
  Description of Artificial Sequence Synthetic oligonucleotide
caccaaagcatggtggcggatcg

SEQ ID NO: 24 - RNA, 23 nt, Artificial Sequence
  Description of Artificial Sequence Synthetic oligonucleotide
caccaaagcaugguggcggaucg

SEQ ID NO: 25 - DNA, 16 bp, Artificial Sequence
  Description of Artificial Sequence Synthetic oligonucleotide
gactgggcctgctgct

SEQ ID NO: 26 - RNA, 16 nt, Artificial Sequence
  Description of Artificial Sequence Synthetic oligonucleotide
gacugggccugcugcu

SEQ ID NO: 27 - DNA, 25 bp, Artificial Sequence
  Description of Artificial Sequence Synthetic oligonucleotide
atcagaggagactgggcctgctgct

SEQ ID NO: 28 - RNA, 25 nt, Artificial Sequence
  Description of Artificial Sequence Synthetic oligonucleotide
aucagaggagacugggccugcugcu

SEQ ID NO: 29 - DNA, 15 bp, Artificial Sequence
  Description of Artificial Sequence Synthetic oligonucleotide
tgctgggaccaccct

SEQ ID NO: 30 - RNA, 15 nt, Artificial Sequence
  Description of Artificial Sequence Synthetic oligonucleotide
ugcugggaccacccu

SEQ ID NO: 31 - DNA, 24 bp, Artificial Sequence
  Description of Artificial Sequence Synthetic oligonucleotide
cagaccacctgctgggaccaccct

SEQ ID NO: 32 - RNA, 24 nt, Artificial Sequence
  Description of Artificial Sequence Synthetic oligonucleotide
cagaccaccugcugggaccacccu

SEQ ID NO: 33 - DNA, 22 bp, Artificial Sequence
  Description of Artificial Sequence Synthetic oligonucleotide
gtggagaggagcagaggttgtc

SEQ ID NO: 34 - RNA, 22 nt, Artificial Sequence
  Description of Artificial Sequence Synthetic oligonucleotide
guggagaggagcagagguuguc

SEQ ID NO: 35 - DNA, 23 bp, Artificial Sequence
  Description of Artificial Sequence Synthetic oligonucleotide
cactgcttaaatacggacgagga

SEQ ID NO: 36 - RNA, 23 nt, Artificial Sequence
  Description of Artificial Sequence Synthetic oligonucleotide
cacugcuuaaauacggacgagga

SEQ ID NO: 37 - DNA, 19 bp, Artificial Sequence
  Description of Artificial Sequence Synthetic oligonucleotide
caggcaccaccactgacct

SEQ ID NO: 38 - RNA, 19 nt, Artificial Sequence
  Description of Artificial Sequence Synthetic oligonucleotide
caggcaccaccacugaccu

SEQ ID NO: 39 - DNA, 23 bp, Artificial Sequence
  Description of Artificial Sequence Synthetic oligonucleotide
ctgttccactggtagcaagatcc

SEQ ID NO: 40 - RNA, 23 nt, Artificial Sequence
  Description of Artificial Sequence Synthetic oligonucleotide
cuguuccacugguagcaagaucc

SEQ ID NO: 41 - DNA, 23 bp, Artificial Sequence
  Description of Artificial Sequence Synthetic oligonucleotide
atgatcatgttcaccctctgcat

SEQ ID NO: 42 - RNA, 23 nt, Artificial Sequence
  Description of Artificial Sequence Synthetic oligonucleotide
augaucauguucacccucugcau

SEQ ID NO: 43 - DNA, 24 bp, Artificial Sequence
  Description of Artificial Sequence Synthetic oligonucleotide
cataacctttgctagcagattgtg

SEQ ID NO: 44 - RNA, 24 nt, Artificial Sequence
  Description of Artificial Sequence Synthetic oligonucleotide
cauaaccuuugcuagcagauugug

SEQ ID NO: 45 - DNA, 24 bp, Artificial Sequence
  Description of Artificial Sequence Synthetic oligonucleotide
ctttgctagcagattgtgaaagtg

SEQ ID NO: 46 - RNA, 24 nt, Artificial Sequence
  Description of Artificial Sequence Synthetic oligonucleotide
cuuugcuagcagauugugaaagug

SEQ ID NO: 47 - DNA, 18 bp, Artificial Sequence
  Description of Artificial Sequence Synthetic oligonucleotide
actacctaaaccacgcca

SEQ ID NO: 48 - RNA, 18 nt, Artificial Sequence
  Description of Artificial Sequence Synthetic oligonucleotide
acuaccuaaaccacgcca

SEQ ID NO: 49 - DNA, 27 bp, Artificial Sequence
  Description of Artificial Sequence Synthetic oligonucleotide
ccctctcacactacctaaaccacgcca

SEQ ID NO: 50 - RNA, 27 nt, Artificial Sequence
  Description of Artificial Sequence Synthetic oligonucleotide
cccucucacacuaccuaaaccacgcca

SEQ ID NO: 51 - DNA, 16 bp, Artificial Sequence
  Description of Artificial Sequence Synthetic oligonucleotide
ctgaggagacagggcc

SEQ ID NO: 52 - RNA, 16 nt, Artificial Sequence
  Description of Artificial Sequence Synthetic oligonucleotide
cugaggagacagggcc

SEQ ID NO: 53 - DNA, 25 bp, Artificial Sequence
  Description of Artificial Sequence Synthetic oligonucleotide
tgcctgaagctgaggagacagggcc

SEQ ID NO: 54 - RNA, 25 nt, Artificial Sequence
  Description of Artificial Sequence Synthetic oligonucleotide
ugccugaagcugaggagacagggcc

SEQ ID NO: 55 - DNA, 16 bp, Artificial Sequence
  Description of Artificial Sequence Synthetic oligonucleotide
accactgacctgggac

SEQ ID NO: 56 - RNA, 16 nt, Artificial Sequence
  Description of Artificial Sequence Synthetic oligonucleotide
accacugaccugggac

SEQ ID NO: 57 - DNA, 25 bp, Artificial Sequence
  Description of Artificial Sequence Synthetic oligonucleotide
tcaggcaccaccactgacctgggac

SEQ ID NO: 58 - RNA, 25 nt, Artificial Sequence
  Description of Artificial Sequence Synthetic oligonucleotide
ucaggcaccaccacugaccugggac

SEQ ID NO: 59 - DNA, 21 bp, Artificial Sequence
  Description of Artificial Sequence Synthetic oligonucleotide
tgaaagtggtattcactgtcc

SEQ ID NO: 60 - RNA, 21 nt, Artificial Sequence
  Description of Artificial Sequence Synthetic oligonucleotide
ugaaagugguauucacugucc

SEQ ID NO: 61 - DNA, 30 bp, Artificial Sequence
  Description of Artificial Sequence Synthetic oligonucleotide
agcagattgtgaaagtggtattcactgtcc

SEQ ID NO: 62 - RNA, 30 nt, Artificial Sequence
  Description of Artificial Sequence Synthetic oligonucleotide
agcagauugugaaagugguauucacugucc

SEQ ID NO: 63 - DNA, 22 bp, Artificial Sequence
  Description of Artificial Sequence Synthetic oligonucleotide
caggatgggctacctgcccgtg

SEQ ID NO: 64 - RNA, 22 nt, Artificial Sequence
  Description of Artificial Sequence Synthetic oligonucleotide
caggaugggcuaccugcccgug

SEQ ID NO: 65 - DNA, 23 bp, Artificial Sequence
  Description of Artificial Sequence Synthetic oligonucleotide
cctactacatcaaccacgagacc

SEQ ID NO: 66 - RNA, 23 nt, Artificial Sequence
  Description of Artificial Sequence Synthetic oligonucleotide
ccuacuacaucaaccacgagacc

SEQ ID NO: 67 - DNA, 27 bp, Artificial Sequence
  Description of Artificial Sequence Synthetic oligonucleotide
cactctgatcgatgcatctgagctctt

SEQ ID NO: 68 - RNA, 27 nt, Artificial Sequence
  Description of Artificial Sequence Synthetic oligonucleotide
cacucugaucgaugcaucugagcucuu

SEQ ID NO: 69 - DNA, 23 bp, Artificial Sequence
  Description of Artificial Sequence Synthetic oligonucleotide
taggcctctcgagctcctcatca

SEQ ID NO: 70 - RNA, 23 nt, Artificial Sequence
  Description of Artificial Sequence Synthetic oligonucleotide
uaggccucucgagcuccucauca

SEQ ID NO: 71 - DNA, 23 bp, Artificial Sequence
  Description of Artificial Sequence Synthetic oligonucleotide
ctctgatacagctcggtcatctt

SEQ ID NO: 72 - RNA, 23 nt, Artificial Sequence
  Description of Artificial Sequence Synthetic oligonucleotide
cucugauacagcucggucaucuu

SEQ ID NO: 73 - DNA, 21 bp, Artificial Sequence
  Description of Artificial Sequence Synthetic oligonucleotide
gaaggcgacaacatggagacc

SEQ ID NO: 74 - RNA, 21 nt, Artificial Sequence
  Description of Artificial Sequence Synthetic oligonucleotide
gaaggcgacaacauggagacc

SEQ ID NO: 75 - DNA, 30 bp, Artificial Sequence
  Description of Artificial Sequence Synthetic oligonucleotide
accgtgctggaaggcgacaacatggagacc

SEQ ID NO: 76 - RNA, 30 nt, Artificial Sequence
  Description of Artificial Sequence Synthetic oligonucleotide
accgugcuggaaggcgacaacauggagacc

SEQ ID NO: 77 - DNA, 15 bp, Artificial Sequence
  Description of Artificial Sequence Synthetic oligonucleotide
tgctgggaccaccct

SEQ ID NO: 78 - RNA, 15 nt, Artificial Sequence
  Description of Artificial Sequence Synthetic oligonucleotide
ugcugggaccacccu

SEQ ID NO: 79 - DNA, 24 bp, Artificial Sequence
  Description of Artificial Sequence Synthetic oligonucleotide
cagaccacctgctgggaccaccct

SEQ ID NO: 80 - RNA, 24 nt, Artificial Sequence
  Description of Artificial Sequence Synthetic oligonucleotide
cagaccaccugcugggaccacccu

SEQ ID NO: 81 - DNA, 26 bp, Artificial Sequence
  Description of Artificial Sequence Synthetic oligonucleotide
actggattaaggagaaaaccaagctg

SEQ ID NO: 82 - RNA, 26 nt, Artificial Sequence
  Description of Artificial Sequence Synthetic oligonucleotide
acuggauuaaggagaaaaccaagcug

SEQ ID NO: 83 - DNA, 26 bp, Artificial Sequence
  Description of Artificial Sequence Synthetic oligonucleotide
tgtgaactggattaaggagaaaacca

SEQ ID NO: 84 - RNA, 26 nt, Artificial Sequence
  Description of Artificial Sequence Synthetic oligonucleotide
ugugaacuggauuaaggagaaaacca

SEQ ID NO: 85 - DNA, 27 bp, Artificial Sequence
  Description of Artificial Sequence Synthetic oligonucleotide
tctatatctaaaaggcaagcatactca

SEQ ID NO: 86 - RNA, 27 nt, Artificial Sequence
  Description of Artificial Sequence Synthetic oligonucleotide
ucuauaucuaaaaggcaagcauacuca

SEQ ID NO: 87 - DNA, 22 bp, Artificial Sequence
  Description of Artificial Sequence Synthetic oligonucleotide
ggcaactagaaggcacagtaga

SEQ ID NO: 88 - RNA, 22 nt, Artificial Sequence
  Description of Artificial Sequence Synthetic oligonucleotide
ggcaacuagaaggcacaguaga

SEQ ID NO: 89 - DNA, 12 bp, Artificial Sequence
  Description of Artificial Sequence Synthetic oligonucleotide
gggggatcagcc

SEQ ID NO: 90 - RNA, 12 nt, Artificial Sequence
  Description of Artificial Sequence Synthetic oligonucleotide
gggggaucagcc

SEQ ID NO: 91 - DNA, 21 bp, Artificial Sequence
  Description of Artificial Sequence Synthetic oligonucleotide
ctgcagccagggggatcagcc

SEQ ID NO: 92 - RNA, 21 nt, Artificial Sequence
  Description of Artificial Sequence Synthetic oligonucleotide
cugcagccagggggaucagcc